SEQUENCE LISTING (26 sequences)

SEQ ID NO: 1 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 2 - DNA, 789 bp, Homo sapiens
  source 1..789: mol_type = other DNA
acacagagagaaaggctaaagttctctggaggatgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgagaccggccagatgaggctggccaagccggggagctgctctctcatgaaacaagagctagaaactcaggatggtcatcttggagggaccaaggggtgggccacagccatggtgggagtggcctggacctgccctgggcacactgaccctgatacaggcatggcagaagaatgggaatattttatactgacagaaatcagtaatatttatatatttatatttttaaaatatttatttatttatttatttaagttcatattccatatttattcaagatgttttaccgtaataattattattaaaaatagcttctaaaaaaaaaaa

SEQ ID NO: 3 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 4 - DNA, 471 bp, Mus musculus
  source 1..471: mol_type = other DNA
atggctcacgagcggaaggctaaggtgctgcgcagaatgtggctgcagaacctgctgttcctgggcatcgtggtgtacagcctgagcgcccccaccagaagccccatcaccgtgaccagaccctggaagcacgtggaggccatcaaggaagctctgaacctgctggacgacatgcccgtgaccctgaacgaggaggtggaggtggtgagcaacgagtttagctttaagaagctgacctgcgtgcagacccggctgaagatcttcgagcagggactgcggggcaactttaccaagctgaagggagccctgaacatgaccgccagctactaccagacctactgccctcccacacccgagaccgactgtgaaacccaggtgaccacctacgccgactttatcgacagcctgaagaccttcctgaccgacatccccttcgagtgtaagaagcccgtgcagaagtgactcgagcgg

SEQ ID NO: 5 - DNA, 440 bp, synthetic construct
  source 1..440: mol_type = other DNA
ctagccaccatgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtgagggcc

SEQ ID NO: 6 - DNA, 574 bp, Homo sapiens
  source 1..574: mol_type = other DNA
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 7 - DNA, 873 bp, Cytomegalovirus
  source 1..873: mol_type = other DNA
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 8 - DNA, 395 bp, unidentified
  source 1..395: mol_type = other DNA
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 9 - DNA, 760 bp, synthetic construct
  source 1..760: mol_type = other DNA
gtaagtatcaaggttacaagacaggtttaaggagaccaatagaaactgggcttgtcgagacagagaagactcttgcgtttctgataggcacgcggccgcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatgcggccgcctattggtcttactgacatccactttgcctttctctccacag

SEQ ID NO: 10 - DNA, 2532 bp, synthetic construct
  source 1..2532: mol_type = other DNA
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacaggttggtgtacagtagcttgctagccaccatgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtgagggccctgtgccttctagttgccagccatctgttgtttgcccctcccctgtgccttccttgaccctggaaggtgccactcccactgtcctttcctaataaaatgaggaaattgcattgcattgtctgagtaggtgtcattctattctggggggtggggtggggcaggacagcaagggggaggattgggaagacaatagcaggcatgcagatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctggtaccttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagcttatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttctaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatggatcccctcctatagtgagttgtattatactatgcagatatactatgccaatgtttaattgtcaa

SEQ ID NO: 11 - DNA, 2723 bp, synthetic construct
  source 1..2723: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagctagccaccatgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtgagggccctgtgccttctagttgccagccatctgttgtttgcccctcccctgtgccttccttgaccctggaaggtgccactcccactgtcctttcctaataaaatgaggaaattgcattgcattgtctgagtaggtgtcattctattctggggggtggggtggggcaggacagcaagggggaggattgggaagacaatagcaggcatgcagatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctggtaccttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagcttatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttctaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatggatcccctcctatagtgagttgtattatactatgcagatatactatgccaatgtttaattgtcaa

SEQ ID NO: 12 - DNA, 600 bp, Woodchuck hepatitis virus
  source 1..600: mol_type = other DNA
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct

SEQ ID NO: 13 - DNA, 526 bp, unidentified
  source 1..526: mol_type = other DNA
accgagctcttacgcgggtcgaagcggagtactgtcctccgagtggagtactgtcctccgagcggagtactgtcctccgagtcgagggtcgaagcggagtactgtcctccgagtggagtactgtcctccgagcggagtactgtcctccgagtcgactctagagggtatataatggatctcgagatatcggagctcgtttagtgaaccgtcagatcgcctggagacgccatccacgctgttttgacctccatagaagacaccgggaccgatccagcctccgcggccgggaacggtgcattggaacgcgcattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccaagcttggtaccgagctcggatccactagtccagtgtggtggaattctgcagatcgaaacgatgatagatccc

SEQ ID NO: 14 - DNA, 1965 bp, unidentified
  source 1..1965: mol_type = other DNA
atggactcccagcagccagatctgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttagaattcccgggtgtcgaccagaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgggtcgacgcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaa

SEQ ID NO: 15 - DNA, 6198 bp, synthetic construct
  source 1..6198: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatttttttgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggaccgagctcttacgcgggtcgaagcggagtactgtcctccgagtggagtactgtcctccgagcggagtactgtcctccgagtcgagggtcgaagcggagtactgtcctccgagtggagtactgtcctccgagcggagtactgtcctccgagtcgactctagagggtatataatgaagcttctgccttctccctcctgtaacgttgagtttgctagccaccatgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtgagggcccacccagctttcttgtacaaagtggtgataatcgaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 16 - DNA, 9064 bp, synthetic construct
  source 1..9064: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccaccatgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattgaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 17 - DNA, 9886 bp, synthetic construct
  source 1..9886: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagactagtgacttggtgagtaggcttcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaaatggcagggaatagtcagatattggatgagacaaagaaatttgaaatggaactattatatgcatcagctggcggccgcgaattcactagtgattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccaatcgaattcccgcggccgcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 18 - DNA, 3384 bp, synthetic construct
  source 1..3384: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgattatgcccctaggaccagaagaaagaagattgcttcgcttgatttggctcctttacagcaccaatccatatccaccaagtggggaagggacggccagacaacgccgacgagccaggagaaggtggagacaacagcaggatcaaattagagtcttggtagaaagactccaagagcaggtgtatgcagttgaccgcctggctgacgaggctcaacacttggctatacaacagttgcctgaccctcctcattcagcttagaatcactagtgaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgtcgaccaattgttgtgtctcaaaatctctgatgttacattgcacaagataaaaatatatcatcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctgttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 19 - DNA, 6264 bp, synthetic construct
  source 1..6264: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgattgccatggcaacatatatccagagagtacagtgcatctcaacatcactactggttgttctcaccacattggtctcgtgtcagattcccagggataggctctctaacataggggtcatagtcgatgaagggaaatcactgaagatagctggatcccacgaatcgaggtacatagtactgagtctagttccgggggtagactttgagaatgggtgcggaacagcccaggttatccagtacaagagcctactgaacaggctgttaatcccattgagggatgccttagatcttcaggaggctctgataactgtcaccaatgatacgacacaaaatgccggtgctccccagtcgagattcttcggtgctgtgattggtactatcgcacttggagtggcgacatcagcacaaatcaccgcagggattgcactagccgaagcgagggaggccaaaagagacatagcgctcatcaaagaatcgatgacaaaaacacacaagtctatagaactgctgcaaaacgctgtgggggaacaaattcttgctctaaagacactccaggatttcgtgaatgatgagatcaaacccgcaataagcgaattaggctgtgagactgctgccttaagactgggtataaaattgacacagcattactccgagctgttaactgcgttcggctcgaatttcggaaccatcggagagaagagcctcacgctgcaggcgctgtcttcactttactctgctaacattactgagattatgaccacaatcaggacagggcagtctaacatctatgatgtcatttatacagaacagatcaaaggaacggtgatagatgtggatctagagagatacatggtcaccctgtctgtgaagatccctattctttctgaagtcccaggtgtgctcatacacaaggcatcatctatttcttacaacatagacggggaggaatggtatgtgactgtccccagccatatactcagtcgtgcttctttcttagggggtgcagacataaccgattgtgttgagtccagattgacctatatatgccccagggatcccgcacaactgatacctgacagccagcaaaagtgtatcctgggggacacaacaaggtgtcctgtcacaaaagttgtggacagccttatccccaagtttgcttttgtgaatgggggcgttgttgctaactgcatagcatccacatgtacctgcgggacaggccgaagaccaatcagtcaggatcgctctaaaggtgtagtattcctaacccatgacaactgtggtcttataggtgtcaatggggtagaattgtatgctaaccggagagggcacgatgccacttggggggtccagaacttgacagtcggtcctgcaattgctatcagacccgttgatatttctctcaaccttgctgatgctacgaatttcttgcaagactctaaggctgagcttgagaaagcacggaaaatcctctcggaggtaggtagatggtacaactcaagagagactgtgattacgatcatagtagttatggtcgtaatattggtggtcattatagtgatcatcatcgtgctttatagactcagaaggtgaaatcactagtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 20 - DNA, 6522 bp, synthetic construct
  source 1..6522: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagcatgtggtctgagttaaaaatcaggagcaacgacggaggtgaaggaccagaggacgccaacgacccccggggaaagggggtgcaacacatccatatccagccatctctacctgtttatggacagagggttagggatggtgataggggcaaacgtgactcgtactggtctacttctcctagtggtagcaccacaaaaccagcatcaggttgggagaggtcaagtaaagccgacacatggttgctgattctctcattcacccagtgggctttgtcaattgccacagtgatcatctgtatcataatttctgctagacaagggtatagtatgaaagagtactcaatgactgtagaggcattgaacatgagcagcagggaggtgaaagagtcacttaccagtctaataaggcaagaggttatagcaagggctgtcaacattcagagctctgtgcaaaccggaatcccagtcttgttgaacaaaaacagcagggatgtcatccagatgattgataagtcgtgcagcagacaagagctcactcagcactgtgagagtacgatcgcagtccaccatgccgatggaattgccccacttgagccacatagtttctggagatgccctgtcggagaaccgtatcttagctcagatcctgaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtggcaaccgggactaggggttatcagctttgctccatgccgactgtagacgaaagaaccgactactctagtgatggtattgaggatctggtccttgatgtcctggatctcaaagggagaactaagtctcaccggtatcgcaacagcgaggtagatcttgatcacccgttctctgcactataccccagtgtaggcaacggcattgcaacagaaggctcattgatatttcttgggtatggtggactaaccacccctctgcagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaataaagagtgcaattggtacaataagtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaagcggccgcgcatgcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtctattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 21 - DNA, 8244 bp, synthetic construct
  source 1..8244: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatttttttgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggacgagcttgcatgcctgcaggtcgaagcggagtactgtcctccgagtttaaaagcggagtactgtcctccgaggatatcagcggagtactgtcctccgagtcgcgaagcggagtactgtcctccgagatcgatgtcgaccccgcccagcgtcttgtcattggcgaattcgaacacgcagatgcagtcggggcggcgcggtccgaggtccacttcgcatattaaggtgacgcgtgtggcctcgaatcgcctggagacgccatccacgctgttttgacctccatagaagacaccgggaccgatccagcctccgcggccgggaacggtgcattggaacgcggattccccgtgttaattaacaggtaagtgtcttcctcctgtttccttcccctgctattctgctcaaccttcctatcagaaactgcagtatctgtatttttgctagcagtaatactaacggttctttttttctcttcacaggccaccaagctaccggtccaccatggactcccagcagccagatctgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttagaattcccgggtgtcgaccagaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgggtcgacgcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaaggatcctccggactagaaaagccgaattctgcaggaattgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggctcgagggggggcccgaaacccgctgatcagcctcgaggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 22 - DNA, 9067 bp, synthetic construct
  source 1..9067: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatttttttgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagaccgcctcagaaggcatgcctgctattgtcttcccaatcctcccccttgctgtcctgccccaccccaccccccagaatagaatgacacctactcagacaatgcaatgcaatttcctcattttattaggaaaggacagtgggagtggcaccttccagggtcaaggaaggcacaggggaggggcaaacaacagatggctggcaactagaaggcacagtcgaggctgatcggatccttaggagctgatctgactcagcagggctgagaagtccatgtccgcaatggaggagaagtcttcatctcctgaaaggaggccattggggagccccggggcccccagtggagcaggagctgggtcggggggcctctgggcccctgtcactaggcgagttatagcctcagggtactccatcagcatgggctcagttgtgtggggggccacaggtatgccctggttcagcagctgctgaaactcggagttgtcgacggatgccaggtctgtgaacacagctgggtctgtgctgttgccaagcaaggcccccaggtcttcatcatcaaactgcagctgcagcagggcctctgacagcgttccttccccagcctgggtgggcttgggggcaggtggggccacagcctgaggagggcctggggctaggactgggacaggggctggggcctgggccagagctgataccatggctggagcaggggcaggggctggagcctggggcaggacttggggaggggccggggccaaggccgaggcctggctgatctgcccagaaggaaacaccatggtgggaaactcatcatagttgatggtgctcagggatgacgtaaagggatagggctggggtgctggcttggggacagaagctgagctgcgggaaggcacagcaatgcgtcgaggtggaggccgggggtcggtgggtccgctgaaaggactcttcttcatgatgctcttgaaggtctcatatgtccttttacgtttctcctcaatccggtgacgatcgtctgtatctggcaggtactggaattccatgggcgtcgacccagcaataacttcagacatcatttctggaaattcaacactcagtgcccgggactggataaatgtattcaagcagtacagatgaagttgtttgacaagatcatgcaagttatcaagaagttttgtaagttgatagaaacgctgtgagctcgacacaactcctttttgcctcaaaccaattgccttgatgagctctctaatgtagcttgacctcatctcctcaaactgggtttgacttcgtagcccttccaaaggaattgtattaagaagtaacaatactttcatacagaggaactcttcttggctaacttgaagcttgacaaactcctgtgggatctgccacatggtaaggcataatgaatagaatgatgattctttcatccgctgttcatttagtattagatcaggtgcaaaatacagcatctgcccactgacgtgtttgtaggatctccatcctagaccaaacaccattaagctcatccaagaatactgaatgagagttatctggtcatcaatatgtaagtttcgaaaacctggcaatgatttagaccacttgactactgaaagaagttgcctctcgcctagttgattaagacttgtcagcaaagaactggaggtgtcaggttttgtgttgtcatgtcctgcatagatcacatctggttcaatgctcattaacaggttgatcagtggtggaatcaactgtatgtcttgacctggtgaaaaagtgaatctctggcttagggcttggctttcatttggaacgcccactggctgtgggagagcaacagcatccagtgctctcacaactctgactttattgaactttttctggtcgacacccgggaattctaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcagatctggctgctgggagtccatggtggaccggtagcttggtggcctgtgaagagaaaaaaagaaccgttagtattactgctagcaaactcacaggagggagaaggcagaagcttgaatgttcacagagactactgcacttatatatggttctcccccaccctggggaaaaaggtggagccagtacaccacatcactttcccagtttacccaagccccaccttctctaggcaccagttcaattgcccacccctccccccaacttctcagggactgtgggccatgtgctctctgcccactgaggggcactcagccctcaagcatgctcttctccactagtgaattcccatggtaatagcaatgactaatacatagatgtactgccaagtaggaaagtcccataaggtcatgtactgggcataatgccaggcaggccatttaccatcattgacatcaatagggggcatacttggcatatgatacacttgatgtactgccaagtgggcagttaccataaatactccacccattgacatcaatggaaagtccctattggcattacatgggaacatacatcattattgacatcattgggcaggggtcattgggcagtcagccaggcaggccatttaccataagttatgtaaccttctgaggcggaaagaaccagctggggctctagggggtatccccaccgagctcttacgcgggtcgaagcggagtactgtcctccgagtggagtactgtcctccgagcggagtactgtcctccgagtcgagggtcgaagcggagtactgtcctccgagtggagtactgtcctccgagcggagtactgtcctccgagtcgactctagagggtatataatgaagcttctgccttctccctcctgtaacgttgagtttgctagccaccatgtggctgcagaacctgctgttcctgggcattgtggtgtacagcctgtctgcccctacaagatcccctatcacagtgaccagaccttggaaacatgtggaagccatcaaagaggccctgaatctgctggatgacatgcctgtgacactgaatgaagaggtggaagtggtgtccaatgagttcagcttcaagaaactgacctgtgtgcagaccaggctgaagatttttgagcagggcctgagaggcaacttcaccaagctgaaaggggctctgaacatgacagccagctactaccagacctactgtcctcctacacctgagacagactgtgaaacccaagtgaccacctatgctgacttcattgacagcctcaagaccttcctgacagacatcccctttgagtgcaagaaacctggccagaagtgagggcccacccagctttcttgtacaaagtggtgataatcgaattcaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 23 - DNA, 5824 bp, synthetic construct
  source 1..5824: mol_type = other DNA
ggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcaacagagatcgatctgtttccttgacactatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccatacaagtcaaaatgcccaagagtcacaaggctattcaagcagacggttggatgtgtcatgcttccaaatgggtcactacttgtgatttccgctggtatggaccgaagtatataacacagtccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaactcaaatcctgcacaacagattcttcatgtttggaccaaatcaacttgtgataccatgctcaaagaggcctcaattatatttgagtttttaatttttatggaattcaccccaccagtgcaggctgcctatcagaaagtggtggctggtgtggctaatgccctggcccacaagtttcactaagctcgcttccttgctgtccaatttctattaaaggttccttggttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagccctgatgcctatgccttattcatccctcagaaaaggattcaagtagaggcttgatttggaggttaaagtttggctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgt

SEQ ID NO: 24 - DNA, 4391 bp, synthetic construct
  source 1..4391: mol_type = other DNA
atgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattga

SEQ ID NO: 25 - DNA, 1738 bp, synthetic construct
  source 1..1738: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccacc

SEQ ID NO: 26 - DNA, 379 bp, Cytomegalovirus
  source 1..379: mol_type = other DNA
gcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

(19) United States
(12) Patent Application Publication
(10) Pub. No.: US 2026/0115317 A1
(43) Pub. Date: Apr. 30, 2026

(54) GENE THERAPY

(71) Applicant: IMPERIAL COLLEGE INNOVATIONS LIMITED, London (GB)

(72) Inventors: Uta GRIESENBACH, London (GB); Eric ALTON, London (GB); Claudia Ivette JUAREZ-MOLINA, London (GB); Helena LUND-PALAU, London (GB); Robyn BELL, London (GB); Cliff MORGAN, London (GB); Robin SHATTOCK, London (GB)

(21) Appl. No.: 19/116,814

(22) PCT Filed: Oct. 2, 2023
(86) PCT No.: PCT/GB2023/052538

§ 371 Date: Mar. 28, 2025

(30) Foreign Application Priority Data
Sep. 30, 2022 (GB) 2214445.5

(51) Int. Cl. A61K 48/00; A01K 67/0276; A61K 9/1272; A61K 9/51; A61K 38/19; A61P 37/04; C07K 16/24; C12N 15/86; C12N 15/88

(52) U.S. Cl. CPC A61K 48/005; A01K 67/0276; A61K 9/1272; A61K 9/5123; A61K 38/193; A61P 37/04; C07K 16/243; C12N 15/86; C12N 15/88; A01K 2227/105; A01K 2267/0325; C12N 2740/10022; C12N 2740/10043; C12N 2740/15022; C12N 2740/15043

(57) ABSTRACT

The present invention relates to gene therapy agents for the treatment of pulmonary alveolar proteinosis (PAP), particularly autoimmune PAP (aPAP). In particular, the present invention relates to gene therapy vectors which drive transient and/or low-level expression of granulocyte-macrophage colony-stimulating factor (GM-CSF), which provide a therapeutic effect without therapy-associated toxicity. The invention further relates to related products and an animal model of aPAP.

### FIELD OF THE INVENTION

The present invention relates to gene therapy agents for the treatment of pulmonary alveolar proteinosis (PAP), particularly autoimmune PAP (aPAP). In particular, the present invention relates to gene therapy vectors which drive transient and/or low-level expression of granulocyte-macrophage colony-stimulating factor (GM-CSF), which provide a therapeutic effect without therapy-associated toxicity. The invention further relates to related products and an animal model of aPAP.

### BACKGROUND TO THE INVENTION

Pulmonary Alveolar Proteinosis (PAP) is a rare autoimmune lung disease with currently insufficient treatment options and no approved pharmacological therapy for clinical use. Most PAP cases are caused by presence of anti-granulocyte-macrophage colony-stimulating factor (GM-CSF) autoantibodies that block surfactant clearance by alveolar macrophages. The current standard of care for aPAP is whole lung lavage (WLL), a technique where the lipoproteinaceous surfactant is washed out of each lung in turn under anaesthesia. WLL is undesirable for several reasons: it is invasive; must be performed at a specialist centre; only treats symptoms; and can lead to complications. Further, about 20% of patients require multiple interventions.

There is currently no approved pharmacological therapy for aPAP. However, recombinant GM-CSF protein has been administered to patients subcutaneously or by aerosol to outcompete the anti-GM-CSF antibodies and restore surfactant clearance. A meta-analysis of these case studies suggests that GM-CSF therapy for aPAP may be effective and that administration by the inhaled route appears to be superior to subcutaneous injection. A recent double-blinded, placebo-controlled trial confirmed that daily administration of inhaled GM-CSF resulted in improvements, albeit modest, in pulmonary gas transfer and functional health status when compared to placebo.

Gene therapy offers several advantages over recombinant protein-based therapies, namely less frequent dosing requirement and more stable steady-state concentrations of therapeutic proteins, which may further enhance the therapeutic index. The UKCF Gene Therapy Consortium has previously generated a lentiviral vector pseudotyped with the F/HN proteins from Sendai virus (rSIV.F/HN) that is specifically designed to achieve high efficiency targeting the lung.

However, a common problem in gene therapy is difficulty in making sufficient protein to reach the therapeutic threshold needed to treat or cure the disease. As such, generating sufficient gene expression is a major barrier to the success of many gene therapies, with existing therapies requiring administration of massive amounts of the gene therapy agent to a patient, over 1 trillion viruses per kg of body mass. For example, Zolgensma is given at 1.1×10¹⁴ viral genomes per kg of body mass. Producing so much virus is expensive, contributing to the $1,000,000 USD cost of gene therapies, and giving so much virus to a person can trigger immune responses that threaten the health of the patient and the efficacy of the therapy. To circumvent these problems, research to-date has focused on gain of function mutations resulting in more potent proteins. Such an approach has been used previously in the gene therapies for haemophilia B (the Padua mutation in Factor IX) and lipoprotein lipase deficiency (the S447X variant of lipoprotein lipase).

However, such conventional approaches and agents are not necessarily applicable to aPAP, because GM-CSF has a narrow therapeutic window.

There is therefore an unmet clinical need for new technologies to successfully treat aPAP. It is an object of the invention to address one or more of these problems. In particular, it is an object of the invention to provide new gene therapy vectors which drive transient and/or low-level expression of GM-CSF, which provide a therapeutic effect without therapy-associated toxicity.

### SUMMARY OF THE INVENTION

At present, there remains a pressing need for technology that allows for gene therapy that can provide GM-CSF in a tightly controlled manner, such that GM-CSF is produced at a concentration falling within a narrow therapeutic window. The present inventors have for the first time demonstrated that transient, low-level expression of GM-CSF can be achieved using regulated expression of GM-CSF using a viral vector, or using a non-viral vector system. Further, the inventors have surprisingly shown that such transient and/or low-level expression of GM-CSF can ameliorate the PAP phenotype in a mouse model of aPAP, with GM-CSF expression stopping at a time before toxicity is reported in the art.

Accordingly, the present invention provides a granulocyte-macrophage colony-stimulating factor (GM-CSF) gene therapy agent for use in the treatment of pulmonary alveolar proteinosis (PAP), wherein said agent transiently expresses GM-CSF within a patient.

Transient GM-CSF protein expression may be expression for six months or less, preferably 4 months or less, more preferably 3 months or less. Said treatment may reduce one or more PAP biomarker selected from: (a) bronchoalveolar lavage fluid (BALF) turbidity; (b) surfactant protein D (SF-D) concentration in the lungs; (c) SF-D concentration in BALF; (d) surfactant deposition in the lungs; and/or (e) lung pathology, which is optionally selected from (i) pulmonary opacities, (ii) pulmonary oedema, and/or (iii) pulmonary consolidation. Alternatively or in addition, said treatment may increase lung function, which may optionally be selected from increasing (i) vital capacity (VC); (ii) forced vital capacity (FVC); and/or (iii) forced expiratory volume (FEV), particularly FEV1; (iv) arterial oxygen tension (Pa,O₂); (v) alveolar to arterial oxygen tension difference (PA-a,O₂); (vi) peak metabolic equivalents (peak METS) and/or (vii) 6-min walk distance (6 MWD), preferably PA-a,O₂.

Said treatment may not be not associated with one or more histopathological change within the patient, said one or more histopathological change optionally being selected from the group consisting of: (a) one or more histopathological change in the lungs, optionally distorted lung architecture, inflammatory cell infiltration of the lung above the PAP phenotype, increased alveolar wall thickness, pulmonary alveolar microlithiasis (PAM) alveoli, PAM bronchi, the presence of neutrophils in the bronchi, consolidation, the presence of giant cells, eosinophilic material and/or oedema; (b) one or more histopathological change in the liver, optionally inflammatory cell infiltration above the PAP phenotype, portal area inflammation, dilated congested sinusoids and/or dilated congested blood vessels; (c) one or more histopathological change in the kidneys, optionally inflammatory cell infiltration above the PAP phenotype, dilated blood vessels, fibrosis, eosinophilic material and/or cysts; and/or (d) one or more histopathological change in the spleen, optionally clusters of megakaryocytes and/or the presence of macrophages.

Said agent may comprise: (a) a non-viral nucleic acid molecule encoding GM-CSF, and a lipid carrier; or (b) a viral vector or non-viral nucleic acid molecule comprising a GM-CSF transgene operably linked to an inducible promoter.

The non-viral nucleic acid molecule may be a plasmid comprising a GM-CSF transgene operably linked to a promoter. The GM-CSF transgene; the promoter; or both the GM-CSF transgene and the promoter may each comprise 10 or fewer CpG dinucleotides, or are CpG dinucleotide free. The plasmid may comprise the GM-CSF transgene operably linked to a promoter selected from the group consisting of a hybrid human CMV enhancer/EF1a (hCEF) promoter, a cytomegalovirus (CMV) promoter, and elongation factor 1a (EF1a) promoter; optionally wherein the plasmid comprises a hybrid human CMV enhancer/EF1a (hCEF) promoter. The non-viral nucleic acid molecule may an mRNA or a self-amplifying RNA (saRNA) encoding GM-CSF. The mRNA may comprise pseudouridine (ψ-UTP), a Cap1 and/or a poly(A) tail of between about 10 to 100 adenosine nucleotides, wherein optionally the mRNA is between about 0.5 kb to about 5 kb in length. The saRNA may comprise ψ-UTP, a Cap1 and/or a poly(A) tail of between about 10 to 100 adenosine nucleotides, wherein optionally the saRNA is between about 9 kb to about 12 kb in length.

The lipid carrier may (i) be a lipid nanoparticle, preferably a liposome; (b) comprise one or more cationic lipid, one or more non-cationic lipid, one or more cholesterol-based lipids and one or more PEG-modified lipids; and/or (c) be GL67A.

Said agent may be a viral vector which is a lentiviral or retroviral vector. The lentiviral or retroviral vector may be: (a) pseudotyped with (i) haemagglutinin-neuraminidase (HN) and fusion (F) proteins from a respiratory paramyxovirus, preferably from a Sendai virus, or (ii) or G glycoprotein from Vesicular Stomatitis Virus (G-VSV); and/or (b) a lentiviral vector selected from the group consisting of a Simian immunodeficiency virus (SIV), a Human immunodeficiency virus (HIV) vector, vector, a Feline immunodeficiency virus (FIV) vector, an Equine infectious anaemia virus (EIAV) vector, and a Visna/maedi virus vector, preferably a SIV vector.

The inducible promoter may be: (i) a steroid-regulated promoter, preferably a mifepristone-regulated promoter; or (ii) a chemically-regulated promoter. Alternatively or in addition (i) the transgene operably linked to an inducible promoter and the transactivator for the inducible promoter are comprised in (i) the same lentiviral or retroviral vector, or (ii) separate lentiviral or retroviral vectors.

The agent for use of the invention may be is formulated for administration to the lungs; optionally wherein the administration is by intratracheal or intranasal instillation, aerosol delivery, nebulization, intravenous injection, direct injection into the lungs.

The agent may be for use in treating autoimmune PAP (aPAP).

The invention also provides a method of treatment of PAP comprising administering a therapeutically effective amount of a GM-CSF gene therapy agent to a patient in need thereof.

The invention further provides the use of a GM-CSF gene therapy agent in the manufacture of a medicament for the treatment of PAP.

The invention also provides a composition comprising: (a) a non-viral nucleic acid molecule encoding GM-CSF, and a lipid carrier; or (b) an viral vector comprising a GM-CSF transgene operably linked to an inducible promoter; and which is formulated for administration to the lungs, such that on administration said non-viral nucleic acid molecule or viral vector is capable of transiently expressing GM-CSF within cells of the lungs. The non-viral vector may be a plasmid as defined herein; or the non-viral vector may be an mRNA or saRNA as defined herein; and preferably the lipid carrier may be as defined herein.

The invention also provides a rodent model for aPAP, wherein said rodent has been passively immunised with anti-GM-CSF antibodies by intranasal administration. In said model: (a) said rodent may be a mouse, optionally a mouse with a C57 black 6 background, a wild-type mouse, or a GM-CSF knock out mouse; (b) the anti-GM-CSF antibodies may be murine anti-GM-CSF antibodies; and/or (c) the model may achieve a BALF concentration of anti GM-CSF antibodies of between about 4-6 μg/mL or greater.

The invention also provides a method of generating a rodent model for aPAP, comprising administration of anti-GM-CSF antibodies to a rodent by intranasal administration. In said method: (a) the rodent may be a mouse, optionally a mouse with a C57 black 6 background, a wild-type mouse, or a GM-CSF knock out mouse; and/or (b) the anti-GM-CSF antibodies may be murine anti-GM-CSF antibodies; and/or (c) the model may achieve a BALF concentration of anti GM-CSF antibodies of between about 4-6 μg/mL or greater.

The invention also provides the use of a rodent model as defined herein for: (a) studying aPAP; and/or (b) studying pharmaceuticals, cell products, biologics or small molecules intended for the treatment of aPAP, optionally studying compositions as defined herein.

### BRIEF DESCRIPTION OF THE DRAWINGS

FIG. 1: (a) Schematic linear representation of the pIC017 hCEF GMCSF plasmid, comprising a mGM-CSF transgene under the control of a hCEF promoter, a bovine growth hormone (BGM) polyA sequence, an R6K origin of replication (comprising CpG dinucleotides) and a kanamycin resistance cassette (also comprising CpG dinucleotides). (b) Schematic linear representation of the pIC098 CMV GMCSF plasmid, comprising a mGM-CSF transgene under the control of a CMV promoter, a bovine growth hormone (BGM) polyA sequence, an R6K origin of replication (comprising CpG dinucleotides) and a kanamycin resistance cassette (also comprising CpG dinucleotides).

FIG. 2: a-i show schematic drawings of exemplary plasmids used for production of the exemplary lentiviral vectors of the invention. (a) Shows a schematic of a lentiviral vector genome plasmid (pDNA1^{ta+}) encoding mGM-CSF under the control of an inducible promoter, and a trans-activator, for lentiviral production for a one vector system (pSIV-V1-GMCSF). (b) Shows a schematic of a lentiviral vector genome plasmid (pDNA1) encoding mGM-CSF under the control of an inducible promoter, for lentiviral production for a two vector system (pSIV-V2-GMCSF). (c) Shows a schematic of a lentiviral vector genome plasmid (pDNA1*) encoding a trans-activator, for lentiviral production for a two vector system (pSIV-V2-Transactivator). (d) Shows a schematic of a plasmid encoding codon optimized SIV Gag and Pol (pDNA2a) for lentiviral production (pGM691). (e) Shows a schematic of a plasmid encoding SIV Gag and Pol (pDNA2a) for lentiviral production (pGM297). (f) Shows a schematic of a plasmid encoding SIV Rev (pDNA2b) for lentiviral production (pGM299). (g) Shows a schematic of a plasmid encoding the fusion protein from Sendai virus (pDNA3a) for lentiviral production (pGM301). (h) Shows a schematic of a plasmid encoding the hemagglutinin-neuraminidase protein from Sendai virus (pDNA3b) for lentiviral production (pGM303). (i) Shows a schematic of a plasmid encoding the VSV glycoprotein (pDNA3) for lentiviral production (pMD2.G).

FIG. 3: Graph showing expression of GM-CSF by a non-viral expression vector at 1 month, 2 months and 6 months after a single treatment (green bars) compared with GM-CSF expression using 1×10⁶ TU/mice lentivirus treated group as reference (grey bar). Data presented as median±IQR (n=7-8/group). Kruskal-Wallis test with Dunnett correction for multiple comparisons compared to the Glux control.* p<0.05.

FIG. 4: Graph showing sustained treatment effect after a single dose of GL67A/mGM-CSF pDNA. GM-CSF knockout mice were treated with GL67A-mGM-CSFpDNA complexes at a dose of 80 μg/mice. Untreated WT are included for reference. Animals were culled 1 to 10 months post-transfection and mGM-CSF expression was quantified in (A) lung homogenate. The effect of mGM-CSF expression on biomarkers of PAP were analyzed (B) BALF turbidity measured by absorbance, (C) SP-D concentration in lung homogenate, (D) surfactant protein D (SP-D) concentration in BALF and (E) surfactant deposition in the alveoli quantified as percentage of PAS-positive alveoli. Data are presented as median±interquartile range (n=3-5 group). Kruskal-Wallis test with Dunnett correction for multiple comparisons of treated to the UT control group. *p<0.05, **p<0.01.

FIG. 5: Graphs showing (A) mGM-CSF expression in lung homogenate; (B) surfactant protein D (SP-D) concentration in lung homogenate; (C) BALF turbidity measured by absorbance; and (D) surfactant deposition in alveoli quantified as a percentage of PAS-positive alveoli; in GM-CSF KO mice treated with either a single dose (×1) or 5 doses (×5) of GL67A-mGM-CSFpDNA complexes (80 μg/mice per dose). Horizontal lines denote median±interquartile range (n=3-5 per group). Kruskal-Wallis test with Dunnett correction for multiple comparisons of treated versus control group. *p<0.05, **p<0.01.

FIG. 6: Graph showing transient mGM-CSF expression using inducible promoter. Fully-differentiated human air liquid interface (ALI) cultures were transduced with 1V-GM-CSF MOI 100 and 2V-GM-CSF transgene:transactivator MOI 100:200 and transgene expression induced with 10⁻⁸ M mifepristone for 48 hours. GM-CSF expression was measured in five consecutive daily apical washes. N=3 ALIs/condition.

FIG. 7: Graph showing GM-CSF expression in mouse lung at day 2 and day 22 following dosing with a pDNA encoding for GM-CSF driven by CMV promotor. GM-CSF expression was measured in the lung homogenate by Elisa and corrected by total protein. Data presented an median±interquartile range. n=7 per group

FIG. 8: (A) SDS-page of anti murine GM-CSF antibody after purification. The antibody exhibits both heavy and light chains. Antibody concentration is 820 ug/ml and endotoxin levels are below recommended levels for animal work (0.67 ng of endotoxin per mg of antibody). (B) Standard curve of anti-GM-CSF antibody (B2.6) measured by ELISA (B2.6 concentration against OD450 nm). Regression formula y=0.0045x²+0.1782x+0.1549 and regression coefficient (R²) of 0.9962 were calculated.

FIG. 9: Passive immunisation of mice with anti-GM-CSF antibody (B2.6). WT mice were treated with different doses of B2.6 antibody 10, 40 or 80 μg/mice. Mice were culled 1 day b) (D1) or 7 days c) (D7) after single dose or 1 day after re-dosing d) (D1 re-admin). Antibody was detected in ELF by Elisa. Black dotted lines show either the minimum literate threshold (4 μg/ml ELF) required for onset of aPAP disease, or the maximum recorded titre reported in aPAP patients. Grey dotted lines represent the median antibody titre in two different aPAP cohorts.

FIG. 10: Passive immunisation of mice with anti-GM-CSF antibody (A7.39). 40 μg/mice were given. Antibody levels were measured 1 day (D1), 7 days (D7) or after re-administration (D1 re-ad) to determine the half-life of the antibody and to elucidate an antibody dose schedule require to maintain the median antibody titre from aPAP patients. Black dotted line shows the minimum threshold (4 μg/ml ELF) required for onset of disease. Grey dotted lines represent the median antibody titre in different aPAP cohorts.

FIG. 11: Graph showing results of in vitro neutralisation of GM-CSF by antibody pair (B2.6 and A7.39). Neutralization of GM-CSF (calculated as percentage of inhibition of FDC-P1 growth with the following formula: [1−(OD of a single well−average OD of control cells grown without GM-CSF)×(average OD of control cells grown with GM-CSF−average OD of control cells grown without GM-CSF)−1]×100) was dose-dependent. Data presented as median±interquartile range. n=6-12 wells per group

FIG. 12: Graph showing expression of luciferase reporter gene using different GL67A/mRNA formulations. Total protein concentration and luciferase expression were quantified. All data is represented as Relative Light Units (RLU)/mg in total protein. Every dot represents an individual well.

### DEFINITIONS

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages, and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

A “vector” or “construct” (sometimes referred to as gene delivery or gene transfer “vehicle”) refers to a macromolecule or complex of molecules comprising a polynucleotide to be delivered to a host cell, either in vitro or in vivo. A vector can be a linear or a circular molecule. A vector of the invention may be viral or non-viral. All disclosure herein in relation vectors of the invention applies equally to viral and non-viral vectors unless otherwise stated. All disclosure in relation to viral vectors of the invention applies equally and without reservation to lentiviral (e.g. SIV) vectors, particularly to lentiviral (e.g. SIV) vectors that are pseudotyped with hemagglutinin-neuraminidase (HN) and fusion (F) proteins from a respiratory paramyxovirus (also referred to herein as SIV F/HN or SIV-FHN).

As used herein, the terms “viral vector”, refer to any viral particle which can be used to deliver genetic material into a target cell, including both in vivo and in vitro delivery. The term “viral vector” encompasses both retroviral and lentiviral vectors. All disclosure herein in relation to viral vectors of the invention applies equally and without reservation to retroviral/lentiviral vectors of the invention, and all disclosure herein in relation to retroviral/lentiviral vectors of the invention applies equally and without reservation to viral vectors of the invention.

As used herein, the terms “retroviral vector” and “retroviral F/HN vector” are used interchangeably to mean a retroviral vector comprising a retroviral RNA sequence and pseudotyped with hemagglutinin-neuraminidase (HN) and fusion (F) proteins from a respiratory paramyxovirus, unless otherwise stated. The terms “lentiviral vector” and “lentiviral F/HN vector” are used interchangeably to mean a lentiviral vector pseudotyped with hemagglutinin-neuraminidase (HN) and fusion (F) proteins from a respiratory paramyxovirus, unless otherwise stated. All disclosure herein in relation to retroviral vectors of the invention applies equally and without reservation to lentiviral vectors of the invention and to SIV vectors that are pseudotyped with hemagglutinin-neuraminidase (HN) and fusion (F) proteins from a respiratory paramyxovirus (also referred to herein as SIV F/HN or SIV-FHN).

The term “intron” as used herein refers to a nucleic acid sequence within a gene that is located between exons. Introns are transcribed along with the exons but are removed from the primary gene transcript by RNA splicing to leave mature mRNA. The removal of introns typically leads to the stabilization of mRNA, increasing the amount of mRNA in the cell.

As used herein, the term “plasmid”, refers to a common type of non-viral vector. A plasmid is an extra-chromosomal DNA molecule separate from the chromosomal DNA which is capable of replicating independently of the chromosomal DNA. Preferably a plasmid is circular and may be double-stranded.

The terms “nucleic acid cassette”, “nucleic acid construct”, “expression cassette” and “nucleic acid expression cassette” are used interchangeably to mean a nucleic acid molecule that is capable of directing transcription. A nucleic acid cassette includes, at the least, a promoter or a structure functionally equivalent to a promoter and a nucleic acid sequence to be transcribed. Thus, a nucleic acid cassette includes, at the least, a promoter or a structure functionally equivalent to a promoter and a nucleic acid sequence encoding a protein of interest. In the present invention, a nucleic acid cassette includes, at the least, a promoter or a structure functionally equivalent to a promoter, a nucleic acid sequence encoding a signal peptide and a nucleic acid encoding a therapeutic protein. A nucleic acid cassette may include additional elements, such as an enhancer, and/or a transcription termination signal.

As used herein the terms “signal peptide”, “signal sequence”, “targeting sequence”, “leader sequence” and “secretory signal” are used interchangeably to mean heterogenous peptide sequences that are found at the N-terminus of secreted proteins that are instrumental in initiating the secretion process. In particular, signal peptides are found in proteins that are targeted to the endoplasmic reticulum and eventually destined to be either secreted or retained in the cell membrane of the cell, particularly as single-pass membrane proteins. Signal peptides are typically removed to produce the mature form of the protein. Signal peptides are normally short peptides, typically about 5 to about 40 amino acids in length, such as about 5 to about 35, or about 10 to about 35 amino acids in length, preferably about 10 to about 30 or about 15 to about 30 amino acids in length. A signal peptide may comprise a core of hydrophobic amino acids, said core typically being about 4 to about 20, such as about 5 to about 20, about 5 to about 16 or about 5 to about 15 amino acids in length). When present, a signal peptide is typically present at the N-terminus of a protein.

As used herein, the terms “transduced” and “modified” are used interchangeably to describe cells which have been modified to express a transgene of interest. Typically the modification occurs through transduction of the cells.

As used herein, the terms “titre” and “yield” are used interchangeably to mean the amount of viral/retroviral/lentiviral (e.g. SIV) vector produced by a method of the invention. Titre is the primary benchmark characterising manufacturing efficiency, with higher titres generally indicating that more viral/retroviral/lentiviral (e.g. SIV) vector is manufactured (e.g. using the same amount of reagents). Titre or yield may relate to the number of vector genomes that have integrated into the genome of a target cell (integration titre), which is a measure of “active” virus particles, i.e. the number of particles capable of transducing a cell. Transducing units (TU/mL also referred to as TTU/mL) is a biological readout of the number of host cells that get transduced under certain tissue culture/virus dilutions conditions, and is a measure of the number of “active” virus particles. The total number of (active+inactive) virus particles may also be determined using any appropriate means, such as by measuring either how much Gag is present in the test solution or how many copies of viral RNA are in the test solution. Assumptions are then made that a lentivirus particle contains either 2000 Gag molecules or 2 viral RNA molecules. Once total particle number and a transducing titre/TU have been measured, a particle:infectivity ratio calculated. Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, lie, Phe or Val), (iii) a cysteine or proline is substituted for, or by, any other residue, or (iv) a residue having a bulky hydrophobic or aromatic side chain (e.g., Val, His, lie or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, or at least about 100%, or at least about 150%, or at least about 200%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. aPAP or any other disease described herein. Preferably said healthy individual(s) is not on medication affecting aPAP and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia.

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, and vice versa.

### Treatment of Pulmonary Alveolar Proteinosis (PAP)

The invention relates to the treatment of Pulmonary Alveolar Proteinosis (PAP). PAP is a pulmonary alveoli-filling disease, characterised by dense phospholipoproteinaceous deposits in the alveoli, cough, and shortness of breath. This disease is often related to impaired processing of pulmonary surfactants by alveolar macrophages, a process dependent on granulocyte-macrophage colony-stimulating factor (GM-CSF). PAP has three distinct aetiologies: hereditary, autoimmune, and secondary. Approximately 90-95% of cases of PAP are of autoimmune aetiology, in which a high level of autoantibodies against GM-CSF neutralise the biologic activity of GM-CSF, thereby causing poor surfactant clearance. The invention relates to the treatment of PAP, particularly autoimmune PAP (aPAP). In aPAP, the minimum threshold of autoantibodies against GM-CSG which may cause disease onset has been described in the art as 4 μg/mL in the epithelial lining fluid (ELF) (see Sakagami et al. Am J Respir Crit Care Med. 2010 Jul. 1; 182(1): 49-61., which is herein incorporated by reference in its entirety).

Treatment according to the present invention provides a clinical benefit to a patient. Treatment according to the present invention may be defined as providing any one or more of a treatment outcome as defined below. These definitions may apply to therapeutic and prophylactic treatments as described herein. These treatment biomarkers (e.g. BALF turbidity; SF-D concentration in the lungs and/or BALF; surfactant deposition; lung pathology, such as (i) pulmonary opacities, (ii) pulmonary oedema, and/or (iii) pulmonary consolidation; and/or lung function, such as (i) VC, (ii) FVC, and/or (iii) FEV (e.g. FEV1)) may be considered as biomarkers for PAP, particularly aPAP.

Treatment of PAP, particularly aPAP, according to the invention may reduce the turbidity of BALF from a patient and/or may reduce the duration of lavage and/or lavage fluid volume required until the BALF becomes clear. In particular, treatment may reduce BALF turbidity, duration of lavage and/or lavage fluid volume by at least 20%, at least 25%, at least 30%, at least 40%, at least 50%, at least 60% or more. Preferably, there is a reduction in BALF turbidity of at least 30%, more preferably at least 40%. The reduction in BALF turbidity may be compared with a suitable control, such as the turbidity of BALF from a healthy individual, or the turbidity of BALF from the patient prior to treatment according to the invention. Any appropriate method may be used to assess or quantify BALF turbidity. Standard techniques are known in the art and can be readily used by one of ordinary skill without undue burden. Byway of non-limiting example, BALF turbidity may be analysed at 600 nm absorbance, or may be judged by eye by the clinical practitioner carrying out the lavage.

Alternatively or in addition, treatment of PAP, particularly aPAP, according to the invention may reduce the concentration of surfactant protein D (SF-D) in the lungs and/or BALF of a patient. In particular, treatment may decrease the concentration of surfactant protein D (SF-D) within the lungs and/or BALF by at least 20%, at least 25%, at least 30%, at least 40%, at least 50%, at least 60% or more. Preferably, there is a reduction in BALF turbidity of at least 30%, more preferably at least 40%. The reduction in SF-D concentration in the lungs and/or BALF of a patient may be compared with a suitable control, such as the SF-D concentration in the lungs and/or BALF of a healthy individual, or the SF-D concentration in the lungs and/or BALF of the patient prior to treatment according to the invention. Any appropriate method may be used to assess or quantify SF-D concentration in the lungs and/or BALF. Standard techniques are known in the art and can be readily used by one of ordinary skill without undue burden. By way of non-limiting example, SF-D concentration in the lungs and/or BALF may be analysed by ELISA.

Alternatively or in addition, treatment of PAP, particularly aPAP, according to the invention may reduce surfactant deposition in the lungs, particularly the alveoli, of a patient. In particular, treatment may reduce surfactant deposition within the lungs, particularly the alveoli, by at least 20%, at least 25%, at least 30%, at least 40%, at least 50%, at least 60% or more. Preferably, there is a reduction in surfactant deposition within the lungs, particularly the alveoli, of at least 30%, more preferably at least 40%. The reduction in surfactant deposition in the lungs of a patient may be compared with a suitable control, such as the surfactant deposition in the lungs of a healthy individual, or the surfactant deposition in the lungs of the patient prior to treatment according to the invention. Standard techniques are known in the art and can be readily used by one of ordinary skill without undue burden. By way of non-limiting example, surfactant deposition in the lungs may be analysed by Periodic acid-Schiff (PAS) stain, which detects polysaccharides and mucosubstances such as surfactant.

Alternatively or in addition, treatment of PAP, particularly aPAP, according to the invention may reduce lung pathology, such as reducing pulmonary opacities, pulmonary oedema and/or pulmonary consolidation in a patient. In particular, treatment may reduce pulmonary opacities by at least 20%, at least 25%, at least 30%, at least 40%, at least 50%, at least 60% or more. Preferably, there is a reduction in pulmonary opacities of at least 30%, more preferably at least 40%. In particular, treatment may reduce pulmonary oedema by at least 20%, at least 25%, at least 30%, at least 40%, at least 50%, at least 60% or more. Preferably, there is a reduction in pulmonary oedema of at least 30%, more preferably at least 40%. In particular, treatment may reduce pulmonary consolidation by at least 20%, at least 25%, at least 30%, at least 40%, at least 50%, at least 60% or more. Preferably, there is a reduction in pulmonary consolidation of at least 30%, more preferably at least 40%. The reduction in lung pathology, such as reducing pulmonary opacities, pulmonary oedema and/or pulmonary consolidation in a patient may be compared with a suitable control, such as the lung pathology, such as pulmonary opacities, pulmonary oedema and/or pulmonary consolidation in the lungs of a healthy individual, or the lung pathology, such as pulmonary opacities, pulmonary oedema and/or pulmonary consolidation in the lungs of the patient prior to treatment according to the invention. Standard techniques are known in the art and can be readily used by one of ordinary skill without undue burden. By way of non-limiting example, lung pathology, such as pulmonary opacities, pulmonary oedema and/or pulmonary consolidation in the lungs may be detected by imaging, such as highly sensitive imaging techniques including computerised tomography (CT) and or magnetic resonance imaging (MRI).

Alternatively or in addition, treatment of PAP, particularly aPAP, according to the invention may increase a patient's lung function. There are numerous metrics for lung functions, including vital capacity (VC), forced vital capacity (FVC), forced expiratory volume (FEV); arterial oxygen tension, Pa,O₂; and alveolar to arterial oxygen tension difference (PA-a,O₂). Other metrics for lung function include peak metabolic equivalents (peak METS) and/or 6-min walk distance (6 MWD). One or more of these parameters may be measured at timed intervals. By way of non-limiting example, FEV over 1 second (FEV1) is particularly preferred. Even relatively small improvements in numerical terms can have a significant impact on patient quality of life. Therefore, treatment may increase VC, FVC and/or FEV (e.g. FEV1) by at least 5%, at least 6%, at least 7%, at least 8%, at least 9%, at least 10%, at least 11%, at least 12%, at least 15%, at least 20%, at least 25% or more. Alternatively or in addition, Pa,O₂ and/or PA-a,O₂ may be increased by at least about 5 mmHg, at least about 6 mmHg, at least about 7 mmHg, at least about 8 mmHg, at least about 9 mmHg, at least about 10 mmHg, at least about 11 mmHg, at least about 12 mmHg, at least about 13 mmHg, at least about 14 mmHg, at least about 15 mmHg, or more. Alternatively or in addition, peak METS may be increased by at least about 2 METS, at least about 3 METs or at least about 4 METS. Alternatively or in addition, 6 MWD may be increased by at least about 100 m, at least about 150 m, at least about 200 m, at least about 250 m, at least about 300 m, or more. Preferably, there is an increase in VC, FVC and/or FEV (e.g. FEV1) of at least 5%, more preferably at least 10%. Alternatively or in addition, Pa,O₂ and/or PA-a,O₂ may preferably be increased by at least about 10 mmHg, or at least about 12 mmHg. Alternatively or in addition, peak METS may preferably be increased by at least about 2 METs. Alternatively or in addition, 6 MWD may preferably be increased by at least about 200 m. The increase in VC, FVC, FEV (e.g. FEV1), Pa,O₂, PA-a,O₂, peak METs and/or 6 MWD may be compared with a suitable control, such as the corresponding parameter measured in a healthy individual, or measured in the patient prior to treatment according to the invention. Standard techniques are known in the art and can be readily used by one of ordinary skill without undue burden. By way of non-limiting example, VC, FVC and/or FEV (e.g. FEV1) may be measured by spirometry.

A suitable control may be used as described herein. By way of non-limiting example, one or more treatment outcome in an individual treated according to the present invention may be compared with a suitable control, such as the same parameter in healthy individual, or the parameter in an individual (typically the same individual) with PAP, particularly aPAP, prior to treatment. Any one or more of these treatment outcomes may be measured at one or more time point following treatment and compared with the corresponding one or more parameter in the patient prior to treatment. By way of non-limiting example, any one or more of these treatment outcomes may be measured at 4 weeks, 8 weeks, 12 weeks, 16 weeks, 20 weeks, 24 weeks, 28 weeks or more, preferably 24 weeks following treatment and compared with the corresponding one or more parameter in the patient prior to treatment.

Any combination of (a) BALF turbidity, or the duration of lavage and/or lavage fluid volume required to result in clear BALF; (b) SF-D concentration in the lungs; (c) SF-D concentration in the BALF; (d) surfactant deposition; (e) lung pathology, such as (i) pulmonary opacities, (ii) pulmonary oedema, and/or (iii) pulmonary consolidation; and/or (f) lung function, such as (i) VC, (ii) FVC, (iii) FEV (e.g. FEV1); (iv) Pa,O₂, (v) PA-a,O₂, (vi) peak METs and/or (vii) 6 MWD; as described above may be assessed, quantified or determined in order to evaluate treatment according to the invention. By way of non-limiting example the following combinations may be used: BALF turbidity, or the duration of lavage and/or lavage fluid volume required to result in clear BALF; and SF-D concentration in the lungs (a+b); BALF turbidity, or the duration of lavage and/or lavage fluid volume required to result in clear BALF; and SF-D concentration in the BALF (a+c); BALF turbidity, or the duration of lavage and/or lavage fluid volume required to result in clear BALF; and surfactant deposition (a+d); BALF turbidity, or the duration of lavage and/or lavage fluid volume required to result in clear BALF; and lung pathology (a+e); BALF turbidity, or the duration of lavage and/or lavage fluid volume required to result in clear BALF; and lung function (a+f); SF-D concentration in the lungs; and SF-D concentration in the BALF (b+c); SF-D concentration in the lungs; and surfactant deposition (b+d); SF-D concentration in the lungs; and lung pathology (b+e); SF-D concentration in the lungs; and lung function (b+f); SF-D concentration in the BALF; and surfactant deposition (c+d); SF-D concentration in the BALF; and lung pathology (c+e); SF-D concentration in the BALF; and lung function (c+f); surfactant deposition; and lung pathology (d+e); surfactant deposition; and lung function (d+f); lung pathology; and lung function (e+f); BALF turbidity, or the duration of lavage and/or lavage fluid volume required to result in clear BALF; SF-D concentration in the lungs and SF-D concentration in the BALF (a+b+c); BALF turbidity, or the duration of lavage and/or lavage fluid volume required to result in clear BALF; SF-D concentration in the lungs; and surfactant deposition (a+b+d); BALF turbidity, or the duration of lavage and/or lavage fluid volume required to result in clear BALF; SF-D concentration in the lungs; and lung pathology (a+b+e); BALF turbidity, or the duration of lavage and/or lavage fluid volume required to result in clear BALF; SF-D concentration in the lungs; and lung function (a+b+f); BALF turbidity, or the duration of lavage and/or lavage fluid volume required to result in clear BALF; SF-D concentration in the BALF; and surfactant deposition (a+c+d); BALF turbidity, or the duration of lavage and/or lavage fluid volume required to result in clear BALF; SF-D concentration in the BALF; and lung pathology (a+c+e); BALF turbidity, or the duration of lavage and/or lavage fluid volume required to result in clear BALF; SF-D concentration in the BALF; and lung function (a+c+f); BALF turbidity, or the duration of lavage and/or lavage fluid volume required to result in clear BALF; surfactant deposition; and lung pathology (a+d+e); BALF turbidity, or the duration of lavage and/or lavage fluid volume required to result in clear BALF; surfactant deposition; and lung function (a+d+f); BALF turbidity, or the duration of lavage and/or lavage fluid volume required to result in clear BALF; lung pathology; and lung function (a+e+f); SF-D concentration in the lungs; SF-D concentration in the BALF; and surfactant deposition (b+c+d); SF-D concentration in the lungs; SF-D concentration in the BALF; and lung pathology (b+c+e); SF-D concentration in the lungs; SF-D concentration in the BALF; and lung function (b+c+f); SF-D concentration in the lungs; surfactant deposition; and lung pathology (b+d+e); SF-D concentration in the lungs; surfactant deposition; and lung function (b+d+f); SF-D concentration in the BALF; surfactant deposition; and lung pathology (c+d+e); SF-D concentration in the lungs; lung pathology; and lung function (b+e+f); SF-D concentration in the BALF; surfactant deposition; and lung function (c+d+f); SF-D concentration in the BALF; lung pathology; and lung function (c+e+f); surfactant deposition; lung pathology; and lung function (d+e+f); BALF turbidity, or the duration of lavage and/or lavage fluid volume required to result in clear BALF; SF-D concentration in the lungs; SF-D concentration in the BALF; and surfactant deposition (a+b+c+d); BALF turbidity, or the duration of lavage and/or lavage fluid volume required to result in clear BALF; SF-D concentration in the lungs; SF-D concentration in the BALF; and lung pathology (a+b+c+e); BALF turbidity, or the duration of lavage and/or lavage fluid volume required to result in clear BALF; SF-D concentration in the lungs; SF-D concentration in the BALF; and lung function (a+b+c+f); BALF turbidity, or the duration of lavage and/or lavage fluid volume required to result in clear BALF; SF-D concentration in the lungs; surfactant deposition; and lung pathology (a+b+d+e); BALF turbidity, or the duration of lavage and/or lavage fluid volume required to result in clear BALF; SF-D concentration in the lungs; surfactant deposition; and lung function (a+b+d+f); BALF turbidity, or the duration of lavage and/or lavage fluid volume required to result in clear BALF; SF-D concentration in the lungs; lung pathology; and lung function (a+b+e+f); BALF turbidity, or the duration of lavage and/or lavage fluid volume required to result in clear BALF; SF-D concentration in the BALF; surfactant deposition; and lung pathology (a+c+d+e); BALF turbidity, or the duration of lavage and/or lavage fluid volume required to result in clear BALF; SF-D concentration in the BALF; surfactant deposition; and lung function (a+c+d+f); BALF turbidity, or the duration of lavage and/or lavage fluid volume required to result in clear BALF; SF-D concentration in the BALF; lung pathology; and lung function (a+c+e+f); BALF turbidity, or the duration of lavage and/or lavage fluid volume required to result in clear BALF; surfactant deposition; lung pathology; and lung function (a+d+e+f); SF-D concentration in the lungs; SF-D concentration in the BALF; surfactant deposition; and lung pathology (b+c+d+e); SF-D concentration in the lungs; SF-D concentration in the BALF; surfactant deposition; and lung function (b+c+d+f); SF-D concentration in the lungs; SF-D concentration in the BALF; lung pathology; and lung function (b+c+e+f); SF-D concentration in the lungs; surfactant deposition; lung pathology; and lung function (b+d+e+f); SF-D concentration in the BALF; surfactant deposition; lung pathology; and lung function (c+d+e+f); BALF turbidity, or the duration of lavage and/or lavage fluid volume required to result in clear BALF; SF-D concentration in the lungs; SF-D concentration in the BALF; surfactant deposition; and lung pathology (a+b+c+d+e); BALF turbidity, or the duration of lavage and/or lavage fluid volume required to result in clear BALF; SF-D concentration in the lungs; SF-D concentration in the BALF; surfactant deposition; and lung function (a+b+c+d+f); BALF turbidity, or the duration of lavage and/or lavage fluid volume required to result in clear BALF; SF-D concentration in the lungs; SF-D concentration in the BALF; lung pathology; and lung function (a+b+c+e+f); BALF turbidity, or the duration of lavage and/or lavage fluid volume required to result in clear BALF; SF-D concentration in the lungs; surfactant deposition; lung pathology; and lung function (a+b+d+e+f); BALF turbidity, or the duration of lavage and/or lavage fluid volume required to result in clear BALF; SF-D concentration in the BALF; surfactant deposition; lung pathology; and lung function (a+c+d+e+f); SF-D concentration in the lungs; SF-D concentration in the BALF; surfactant deposition; lung pathology; and lung function (b+c+d+e+f); and BALF turbidity, or the duration of lavage and/or lavage fluid volume required to result in clear BALF; SF-D concentration in the lungs; SF-D concentration in the BALF; surfactant deposition; lung pathology; and lung function (a+b+c+d+e+f) may be used. In any combination above where lung pathology (e) is assessed, quantified or determined, the pathology assessed, quantified or determined may be selected from (i) pulmonary opacities, (ii) pulmonary oedema, (iii) pulmonary consolidation, (iv) pulmonary opacities and pulmonary oedema, (v) pulmonary opacities and pulmonary consolidation, (vi) pulmonary oedema and pulmonary consolidation; or (vii) pulmonary opacities, pulmonary oedema and pulmonary consolidation. Alternatively or in addition, in any combination above where lung function (f) is assessed, quantified or determined, the function assessed, quantified or determined may be selected from (i) VC, (ii) FVC, (iii) FEV (e.g. FEV1), (iv) Pa,O₂, (v) PA-a,O₂, (vi) peak METs; (vii) 6 MWD; or any combination thereof, with PA-a,O₂ or a combination comprising PA-a,O₂ being preferred.

Treatment of PAP, particularly aPAP, according to the invention is typically not associated with one or more histopathological change within the patient. Non-limiting examples of such histopathological changes include (a) one or more histopathological change in the lungs; (b) one or more histopathological change in the liver; (c) one or more histopathological change in the kidneys; and/or (d) one or more histopathological change in the spleen. Treatment of PAP, particularly aPAP, according to the invention may not be associated with any combination of (a), (b), (c) and/or (d).

Histopathological change in the lungs that are typically not associated with treatment according to the invention may include one or more of distorted lung architecture, inflammatory cell infiltration of the lung above the PAP phenotype, increased alveolar wall thickness, pulmonary alveolar microlithiasis (PAM) alveoli, PAM bronchi, the presence of neutrophils in the bronchi, consolidation, the presence of giant cells, eosinophilic material and/or oedema. These histopathological changes may be assessed or determined by any appropriate means, including direct and indirect assessment and/or quantification, such as by imaging (e.g. by CT scan), lung function test or histological analysis, as described herein.

Histopathological change in the liver that are typically not associated with treatment according to the invention may include one or more of inflammatory cell infiltration above the PAP phenotype (e.g. as assessed or quantified in the patient prior to treatment), portal area inflammation, dilated congested sinusoids and/or dilated congested blood vessels. These histopathological changes may be assessed or determined by any appropriate means, including direct and indirect assessment and/or quantification, such as by imaging (e.g. by CT scan), liver function tests or histological analysis, as described herein.

Histopathological change in the kidneys that are typically not associated with treatment according to the invention may include one or more of inflammatory cell infiltration above the PAP phenotype (e.g. as assessed or quantified in the patient prior to treatment), dilated blood vessels, fibrosis, eosinophilic material and/or cysts. These histopathological changes may be assessed or determined by any appropriate means, including direct and indirect assessment and/or quantification, such as by imaging (e.g. by CT scan), kidney function tests or histological analysis, as described herein.

Histopathological change in the spleen that are typically not associated with treatment according to the invention may include one or more of clusters of megakaryocytes and/or the presence of macrophages. These histopathological changes may be assessed or determined by any appropriate means, including direct and indirect assessment and/or quantification, such as by imaging (e.g. by CT scan), splenic function tests or histological analysis, as described herein.

### Expression of GM-CSF

The present invention provides gene therapy vectors which are capable of expressing GM-CSF within a target cell, as described herein. An exemplary GM-CSF is human GM-CSF, which has UniProt Accession No. P04141 (version 1, deposited 1 Nov. 1986, accessed 25 Sep. 2022), or SEQ ID NO: 1. The therapeutic GM-CSF protein may be encoded by the gene CSF2. An example of the human CSF2 transgene is given in GenBank Accession No. M11220.1 (version 1, deposited 8 Nov. 1994, accessed 25 Sep. 2022), which is SEQ ID NO: 2. A further exemplary GM-CSF is mouse GM-CSF, which has UniProt Accession No. P01587 (version 1, deposited 1 Apr. 1988, accessed 25 Sep. 2022), or SEQ ID NO: 3. An example of the mouse CSF2 transgene is given in GenBank Accession No. AY950559.1 (version 1, deposited 19 Dec. 2026, accessed 29 Sep. 2022), which is SEQ ID NO: 4, another example is SEQ ID NO: 5. Preferably the GM-CSF is human GM-CSF (hGM-CSF). Preferably the CSF2 transgene is human CSF2. Variants thereof (as described therein) are also included, particularly variants with at least 90% (such as at least 90, 92, 94, 95, 96, 97, 98, 99 or 100%) to SEQ ID NO: 1, 2, 3, 4 or 5, preferably 1 or 2. Any reference herein to GM-CSF protein may refer to the GM-CSF of SEQ ID NO: 1 or 3, preferably 1, or a functional fragment and/or variant thereof. Any reference herein to a GM-CSF transgene may refer to the GM-CSF transgene of SEQ ID NO: 2, 4 or 5, preferably 2, or a functional fragment and/or variant thereof.

The therapeutic window (also referred to interchangeably herein as the toxicity/efficacy window) is the concentration range of a drug which achieves a therapeutic effect. Below this range there is little or no therapeutic benefit, and above this range the toxicity occurs at an unacceptable level. The therapeutic window for GM-CSF is narrow. This is evidenced by the fact that in GM-CSG knock-out mice, the therapeutic window has been calculated to be in the range of 1×10⁵ TU to less than 1×10⁶ TU using a lentiviral vector (rSIV.F/HN-mGM-CSF).

The prevailing teaching in gene therapy is that large numbers of gene therapy agent must be delivered to achieve a therapeutic effect, driving research to achieve this aim, including increasing vector yield, increasing transgene expression from a vector and introducing gain of function mutations to increase potency of therapeutic proteins.

In contrast to this standard teaching in the art, for the treatment of PAP, particularly aPAP, it is necessary to express GM-CSF within a patient within this narrow therapeutic window. Therefore, the conventional teaching and gene therapy vectors are not suitable for this indication. Preferably, it is the level of free GM-CSF that must be present within a narrow therapeutic window. By free GM-CSF, it is meant GM-CSF that is not neutralised by autoimmune antibodies against GM-CSF. Methods for determining the neutralisation of GM-CSF are routine to one of skill in the art, and are exemplified herein, such as the neutralisation assay described in Example 8. In aPAP, the autoimmune antibodies against GM-CSF may neutralise a proportion of the GM-CSF that is administered to a patient, such that not all the GM-CSF administered is available to perform its physiological function. The levels of autoimmune antibodies against GM-CSF may vary between patients. Typically the invention seeks to provide sufficient GM-CSF such that the free concentration of GM-CSF in a patient falls within the narrow therapeutic window, resulting a therapeutic benefit without the histopathological changes associated with administration of high and/or sustained doses of GM-CSF. Accordingly, any reference herein to the therapeutic window of GM-CSF applies equally and without reservation to the therapeutic window of free GM-CSF.

Instead, the inventors are the first to appreciate that transient and/or low levels of GM-CSF (particularly free GM-CSF) expression can provide a therapeutic benefit without the problems usually associated with GM-CSF expression at higher levels and/or over a longer period of time. In the present application, the present inventors are the first to provide gene therapy agents which are capable of driving GM-CSF expression (particularly free GM-CSF) within the narrow therapeutic window. In particular, the gene therapy agents of the invention allow for the duration of expression of GM-CSG within a patient's cells and/or the concentration of GM-CSF (particularly free GM-CSF) expressed within a patient's cells, to be carefully controlled, allowing transient and/or low-level expression, such that GM-CSF (particularly free GM-CSF) is expressed within a narrow toxicity/efficacy window.

Thus, a gene therapy agent of the invention is typically able to transiently express GM-CSF within a patient (i.e. within cells of the patient into which the agent is introduced). Transient expression of GM-CSF may be defined as expression of six months or less, such as five months or less, four months or less, three months or less, two months or less, one more or less, less than three weeks, less than two weeks or less. In some preferred embodiments, transient expression of GM-CSF is for between about 1-6 months, such as between about 1-4 months, 1-3 months, 1-2 months, 1 week-4 months, 1 week-4 months, 1 week-3 months, 1 week-2 months, or 1 week-1 month. In some preferred embodiments, transient expression of GM-CSF is for three months or less.

Reference herein to expression of GM-CSF applies equally and without reservation to both expression of the GM-CSF transgene and expression of the encoded GM-CSF protein, unless expressly stated to the contrary. Expression levels of the GM-CSF transgene and/or the encoded GM-CSF protein of the invention may be measured in the lung tissue, epithelial lining fluid and/or serum/plasma as appropriate. A therapeutic expression level may therefore refer to the concentration in the lung, epithelial lining fluid and/or serum/plasma. As described herein, in healthy individuals, the concentration of GM-CSF is typically low, or even below the lower limit of detection using standard assays (e.g. ELISA or other standard protein quantification assay). Therefore, the duration of transient GM-CSF (particularly free GM-CSF) expression according to the invention may be defined as the time for which GM-CSF protein can be detected, or the time for which one or more of the treatment outcomes as defined herein is observed.

As described herein, viral vectors of the invention, particularly retroviral/lentiviral (e.g. SIV) vectors of the invention can integrate into the genome of target cells within a patient. Once integrated, these viral vectors, particularly these retroviral/lentiviral (e.g. SIV) vectors are retained within the genome of the target cell for the life of the cell. Accordingly, whilst these viral vectors, particularly these retroviral/lentiviral (e.g. SIV) vectors may be used to drive transient expression of GM-CSF, the vector is typically present (integrated within the genome of) the target cell for longer than the duration of expression. By way of non-limiting example, a viral vector, particularly a retroviral/lentiviral (e.g. SIV) vector, may be present within a target cell for at least 180 days, at least 250 days, at least 360 days, at least 450 days, at least 720 days or more. Using an inducible promoter as described herein, such a viral vector, particularly a retroviral/lentiviral (e.g. SIV) vector may be used to transiently express GM-CSF for a period of six months or less, such as five months or less, four months or less, three months or less, two months or less, one more or less, less than three weeks, less than two weeks or less, preferably three months or less, as described herein.

As described herein, expression of GM-CSF may be induced a single time using an inducible promoter of the invention (whether in a viral/non-viral gene therapy agent). Where a gene therapy agent is retained within a target cell for a prolonged period of time, such as typically the case for a viral vector, particularly a retroviral/lentiviral (e.g. SIV) vector as described herein, expression of GM-CSF may be induced multiple times using the inducible promoter, such as 2, 3, 4, 5, 6, 7, 8, 9 10 or more times.

Accordingly, a gene therapy agent, may be administered a single time, be retained within the target cell, and then used to express GM-CSF in short bursts. This can allow the concentration of GM-CSF (particularly free GM-CSF) to be maintained within the narrow therapeutic window, achieving a therapeutic effect for the patient, whilst reducing and/or eliminating histopathological changes within the patient that are normally associated with prolonged and/or high levels of GM-CSF expression. Without being bound by theory, viral gene therapy agents, such as viral vector, particularly a retroviral/lentiviral (e.g. SIV) vector of the invention, may typically be used for repeat administration, as they do integrate into the genome of target cells and so are retained within the target cells over a prolonged period of time.

Alternatively, repeated doses of a gene therapy agent may be used. Such repeated doses may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated. The frequency of repeated doses may be determined such that the concentration of GM-CSF expressed by the gene therapy agent (particularly free GM-CSF) is maintained within the therapeutic window. Where repeated doses are used, the gene therapy agent may express GM-CSF for as long as it is retained by the target cell. One the gene therapy agent is eliminated (e.g. by degradation) from the target cell, expression of GM-CSF by the vector will cease. Without being bound by theory, non-viral gene therapy agents, such as non-viral nucleic acid molecules, including plasmids, mRNA or self-replicating RNA molecules may typically be used for repeat administration, as they do not integrate into the genome of target cells and so are eliminated from the target cells over time. Repeated administration may be beneficial when a patient has autoimmune antibodies against GM-CSF, which would otherwise neutralise some or all of the GM-CSF expressed by a single administration of a gene therapy agent of the invention, resulting in the level of free GM-CSF falling below the therapeutic window.

Accordingly, the gene therapy agents of the invention are capable of producing repeatable, carefully controlled expression of GM-CSF (particularly free GM-CSF) within its narrow therapeutic window, particularly in airway cells. Further, the transient expression of GM-CSF can be achieved without inducing an undue immune response and whilst reducing and/or eliminating histopathological changes within the patient that are normally associated with prolonged and/or high levels of GM-CSF expression.

### Non-Viral Nucleic Acids

The gene therapy agent of the invention may be a non-viral nucleic acid molecule which encodes GM-CSF. Typically said non-viral nucleic acid molecule is administered with a lipid carrier, as defined herein.

The nucleic acid of the nucleic acid may be as defined herein. The nucleic acid may comprise DNA and/or RNA. Non-limiting examples of non-viral nucleic acid molecules include plasmids, mRNA, and self-amplifying RNA (saRNA), as described herein. Any and all disclosure herein in relation to non-viral nucleic acid molecules of the invention applies equally and without reservation to plasmids, mRNA and/or saRNA molecules of the invention, unless expressly stated to the contrary.

A non-viral nucleic acid molecule may be a DNA molecule or vector, such as a DNA plasmid. A non-viral nucleic acid molecule may be an RNA molecule or vector, such as a mRNA vector or a self-amplifying RNA vector. The DNA and/or RNA vector(s) of the invention may be capable of expression in eukaryotic and/or prokaryotic cells. Typically, the DNA and/or RNA vector(s) is capable of expression in a cell of a patient, for example, a cell of a mammalian or avian subject to be immunised.

A non-viral nucleic acid molecule may be a phage vector, such as an AAV/phage hybrid vector as described in Hajitou et al., Cell 2006; 125(2) pp. 385-398; herein incorporated by reference.

Typically, in a DNA vector of the invention, the GM-CSF transgene is operably linked to a suitable promoter, as described herein. The polynucleotide may also be linked to a suitable terminator sequence. Suitable promoter and terminator sequences are well known in the art. The choice of promoter will depend on where the ultimate expression of the polynucleotide will take place. In general, constitutive promoters are preferred, but inducible promoters may likewise be used. The construct produced in this manner includes at least one part of a vector, in particular, regulatory elements.

Thus, a DNA vector of the invention typically comprises a GM-CSF transgene operably linked to a promoter. The promoter may be an inducible promoter as described herein or a non-inducible promoter. Non-limiting examples of (non-inducible) promoters are disclosed herein in the context of plasmids of the invention. For the avoidance of doubt, the promoters disclosed in the context of plasmids may be operably linked to a GM-CSF transgene in any other type of DNA vector of the invention. Further, any and all disclosure herein of DNA vectors (e.g. plasmids) of the invention applies equally and without reservation to DNA vectors (e.g. plasmids) in which the GM-CSF transgene is operably linked to an inducible promoter, unless expressly stated to the contrary.

The non-viral nucleic acid molecule is preferably capable of expressing a GM-CSF transgene in a given host cell. Any appropriate host cell may be used, such as mammalian, bacterial, insect, yeast, and/or plant host cells. In addition, cell-free expression systems may be used. Such expression systems and host cells are standard in the art. Typically the non-viral nucleic acid molecule is capable of expressing a GM-CSF transgene within a target cell in a patient. Non-limiting examples of suitable target cells within the lungs and airways of a patient include basal cells and submucosal gland duct cells in the upper airways, club cells and neuroendocrine cells in the bronchiolar airways, bronchioalveolar stem cells in the terminal bronchioles, type II pneumocytes in the alveoli, submucosal acinar cells, ionocytes, and type I pneumocytes.

The non-viral nucleic acid molecules of the invention may be made using any suitable process known in the art. Thus, the nucleic acid molecules may be made using chemical synthesis techniques. Alternatively, the nucleic acid molecules of the invention may be made using molecular biology techniques. Non-viral nucleic acid molecules of the present invention may be designed in silico, and then synthesised by conventional polynucleotide synthesis techniques.

As described herein, the gene therapy agents of the invention, including non-viral nucleic acid molecules of the invention, allow for the duration of expression of GM-CSG within a patient's cells and/or the concentration of GM-CSF expressed within a patient's cells, to be carefully controlled, allowing transient and/or low-level expression, such that GM-CSF is expressed within a narrow toxicity/efficacy window.

Thus, a non-viral nucleic acid molecule of the invention is typically able to transiently express GM-CSF within a patient (i.e. within cells of the patient into which the agent is introduced), as defined herein. In some preferred embodiments, transient expression of GM-CSF is for three months or less.

As described herein, expression of GM-CSF may be induced a single time from a non-viral nucleic acid molecule of the invention. Said expression from a DNA vector may comprise transcription from an inducible or non-inducible promoter, as described herein. Expression of GM-CSF may be induced multiple times from a non-viral nucleic acid molecule of the invention using an inducible promoter, such as 2, 3, 4, 5, 6, 7, 8, 9 10 or more times.

Accordingly, a non-viral nucleic acid molecule of the invention may be administered a single time, be retained within the target cell, and then used to express GM-CSF in short bursts, as described herein. Typically for a non-viral nucleic acid molecule of the invention, repeated doses of said non-viral nucleic acid molecule may be used, as described herein. In particular, the frequency of repeated doses may be determined such that the concentration of GM-CSF expressed by the non-viral nucleic acid molecule of the invention (particularly free GM-CSF) is maintained within the therapeutic window.

A non-viral nucleic acid molecule of the invention may optionally be codon optimised for expression in a particular cell type, for example, eukaryotic cells (e.g. mammalian cells, yeast cells, insect cells or plants cells) or prokaryotic cells (e.g. E. coli). The term “codon optimised” refers to the replacement of at least one codon within a base polynucleotide sequence with a codon that is preferentially used by the host organism in which the polynucleotide is to be expressed. Typically, the most frequently used codons in the host organism are used in the codon-optimised polynucleotide sequence. Methods of codon optimisation are well known in the art.

It will be understood by a skilled person that numerous different polynucleotides can encode the same polypeptide as a result of the degeneracy of the genetic code. It is also understood that skilled persons may, using routine techniques, make nucleotide substitutions that do not affect the polypeptide sequence encoded by the nucleic acid molecules to reflect the codon usage of any particular host organism in which the polypeptides are to be expressed. Therefore, unless otherwise specified, a nucleic acid that encodes GM-CSF according to the invention includes all polynucleotide sequences that are degenerate versions of each other and that encode the same amino acid sequence. A DNA molecule of the invention typically comprises a promoter operably linked to the nucleic acid sequence encoding GM-CSF. By operably linked, it is meant that the promoter is configured to express the nucleic acid sequence encoding the signal peptide and/or the nucleic acid sequence encoding GM-CSF.

The (non-viral) nucleic acid molecules of the invention may include at least one part of a vector, in particular, regulatory elements. By way of non-limiting example, the promoter within a DNA molecule of the invention may be used to express more than one polypeptide, including one or more therapeutic proteins in addition to GM-CSF. Thus, the DNA molecule of the invention may comprise a nucleic acid sequence which, when transcribed, gives rise to multiple polypeptides, for instance a transcript may contain multiple open reading frames (ORFs) and also one or more Internal Ribosome Entry Sites (IRES) to allow translation of ORFs after the first ORF. A transcript may be polycistronic, i.e. it may be translated to give a polypeptide which is subsequently cleaved to give a plurality of polypeptides. Alternatively, a DNA molecule of the invention may comprise multiple promoters and hence give rise to a plurality of transcripts and hence a plurality of polypeptides, including a plurality of therapeutic proteins, including GM-CSF. Nucleic acids may, for instance, express one, two, three, four or more polypeptides via a promoter or promoters.

A (non-viral) nucleic acid molecule of the invention may comprise one or more translation initiation sequences (TIS). Translation initiation plays an important role in mRNA translation, canonically a methionyl tRNA unique for initiation (Met-tRNAi) identifies the AUG start codon and triggers the downstream translation process. Non-canonical start codons (e.g. CUG for valyl-tRNA)/TIS may also be used.

A DNA molecule of the present invention may comprise at least one termination signal. A “termination signal” or “terminator” is comprised of the DNA sequences involved in specific termination of an RNA transcript by an RNA polymerase. Thus, a termination signal that ends the production of an RNA transcript is contemplated according to the present invention. A terminator may be necessary in vivo to achieve desirable message levels. In eukaryotic systems, a terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, when the nucleic acid is for expression in eukaryotes, a terminator typically comprises a signal for the cleavage of the RNA, and it is preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

A non-viral nucleic acid molecule of the invention (e.g. a plasmid), or part thereof may be codon-optimised. By way of non-limiting example, the GM-CSF transgene may be codon-optimised and/or the promoter may be codon-optimised, or the entire molecule may be codon-optimised.

A non-viral nucleic acid molecule of the invention (e.g. a plasmid) or part thereof may be modified to reduce the CpG dinucleotide content. Thus, the non-viral nucleic acid molecule of the invention (e.g. a plasmid) or part thereof may have low or no CpG dinucleotide content. By low CpG content, it is meant 20 or fewer, 15 or fewer, 10 or fewer, 5 or fewer CpG dinucleotides (e.g. 20, 19, 18, 17, 16, 15, 14, 13, 12, 11, 10, 9, 8, 7, 6, 5, 4, 3, 2, 1 or 0 CpG dinucleotides). The non-viral nucleic acid molecule of the invention (e.g. a plasmid) or part thereof may have all CG dinucleotides replaced with any one of AG, TG or GT. Thus, the non-viral nucleic acid molecule of the invention (e.g. a plasmid) or part thereof may be CpG-free.

The GM-CSF transgene within a non-viral nucleic acid molecule of the invention (e.g. a plasmid) may have low CpG dinucleotide content as defined herein, preferably the GM-CSF transgene comprises 10 or fewer CpG dinucleotides, or is CpG dinucleotide free. Alternatively or in addition, the promoter within a non-viral nucleic acid molecule of the invention (e.g. a plasmid) may have low CpG dinucleotide content as defined herein, preferably the promoter comprises 10 or fewer CpG dinucleotides, or is CpG dinucleotide free. Preferably both the GM-CSF transgene and the promoter within a non-viral nucleic acid molecule of the invention (e.g. a plasmid) have low CpG dinucleotide content as defined herein, preferably both the GM-CSF transgene and the promoter each comprise 10 or fewer CpG dinucleotides, or are CpG dinucleotide free.

A nucleic acid of the invention may be used in the production of a retroviral/lentiviral (e.g. SIV) vector, as described herein. By way of non-limiting example, a non-viral nucleic acid of the invention may be a plasmid which may be used in the treatment of PAP as described herein, or used in the manufacture of a viral/retroviral/lentiviral (e.g. SIV) vector of the invention. A nucleic acid of the invention may be comprised in a viral/retroviral/lentiviral (e.g. SIV) vector.

Typically the non-viral nucleic acids of the invention are capable of expressing the therapeutic protein in airway cells (as described herein).

Non-viral nucleic acid molecules cannot replicate in the subject to be treated, as they lack the viral genetic material which hijacks the body's normal production machinery. However they are capable of replicating in appropriate host cells, such as yeasts or bacteria including E. coli, and particularly airway cells as defined herein.

### Plasmids

The term “plasmid” as used herein refers to a construction comprised of genetic material designed to direct transformation of a targeted cell. The plasmid contains a plasmid backbone. A “plasmid backbone” as used herein contains multiple genetic elements positionally and sequentially oriented with other necessary genetic elements such that the nucleic acid in the nucleic acid can be transcribed and when necessary translated in the transfected cells.

The plasmid backbone can contain one or more unique restriction sites within the backbone. The plasmid may be capable of autonomous replication in a defined host or organism such that the cloned sequence is reproduced. The plasmid can confer some well-defined phenotype on the host organism which is either selectable or readily detected. The plasmid or plasmid backbone may have a linear or circular configuration. The components of a plasmid can contain, but is not limited to, a DNA molecule incorporating: (1) the plasmid backbone; (2) a sequence encoding a signal peptide; (3) a sequence encoding GM-CSF and optionally one or more additional therapeutic protein; and (4) regulatory elements for transcription, translation, RNA stability and replication

The purpose of the plasmid in human gene therapy for the efficient delivery of nucleic acid sequences to, and expression of therapeutic proteins in, a cell or tissue. In particular, the purpose of the plasmid is to achieve high copy number, avoid potential causes of plasmid instability and provide a means for plasmid selection. As for expression, a nucleic acid of the invention contains the necessary elements for expression of the GM-CSF transgene comprised in the nucleic acid. Expression includes the efficient transcription of an inserted gene, nucleic acid sequence, or nucleic acid within the plasmid.

Thus, a plasmid of the invention typically comprises a GM-CSF transgene operably linked to a promoter. The promoter may be an inducible promoter as described herein. Any and all disclosure herein of plasmids of the invention applies equally and without reservation to plasmids in which the GM-CSF transgene is operably linked to an inducible promoter, unless expressly stated to the contrary.

Alternatively, the promoter may be a (non-inducible) promoter which is capable of expressing GM-CSF within one or more target cell type. Non-limiting examples of suitable target cells within the lungs and airways of a patient include basal cells and submucosal gland duct cells in the upper airways, club cells and neuroendocrine cells in the bronchiolar airways, bronchioalveolar stem cells in the terminal bronchioles, type II pneumocytes in the alveoli, submucosal acinar cells, ionocytes, and type I pneumocytes.

Non-limiting examples of promoters which may used according to the invention, particularly which may be operably linked to a GM-CSF transgene in a non-viral nucleic acid molecule (e.g. plasmid) of the invention, include a hybrid human CMV enhancer/EF1a (hCEF) promoter, a cytomegalovirus (CMV) promoter, and elongation factor 1a (EF1a) promoter. Preferably the non-viral nucleic acid molecule (e.g. plasmid) comprises a hybrid human CMV enhancer/EF1a (hCEF) promoter.

A preferred example of an hCEF promoter sequence of the invention is provided by SEQ ID NO: 6. Alternatively, the promoter may be a CMV promoter. An example of a CMV promoter sequence is provided by SEQ ID NO: 26 or 7, preferably SEQ ID NO: 26. The promoter may be a human elongation factor 1a (EF1a) promoter. An example of a EF1a promoter is provided by SEQ ID NO: 8. Other promoters for transgene expression are known in the art and their suitability for the non-viral nucleic acid molecules (e.g. plasmids) of the invention determined using routine techniques known in the art. Non-limiting examples of other promoters include UBC and UCOE. As described herein, the promoter may be modified to further regulate expression of the transgene of the invention.

The promoter included in the non-viral nucleic acid molecule (e.g. plasmid) of the invention may be specifically selected and/or modified to further refine regulation of expression of the GM-CSF gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90(12): 1487-96), which is herein incorporated by reference in its entirety.

A plasmid of the invention, or part thereof may be codon-optimised. By way of non-limiting example, the GM-CSF transgene may be codon-optimised and/or the promoter may be codon-optimised, or the entire plasmid may be codon-optimised.

A plasmid of the invention or part thereof may be modified to reduce the CpG dinucleotide content. Thus, the plasmid of the invention or part thereof may have low or no CpG dinucleotide content. By low CpG content, it is meant 20 or fewer, 15 or fewer, 10 or fewer, 5 or fewer CpG dinucleotides (e.g. 20, 19, 18, 17, 16, 15, 14, 13, 12, 11, 10, 9, 8, 7, 6, 5, 4, 3, 2, 1 or 0 CpG dinucleotides). The plasmid of the invention or part thereof may have all CG dinucleotides replaced with any one of AG, TG or GT. Thus, the plasmid of the invention or part thereof may be CpG-free.

The GM-CSF transgene within a plasmid of the invention may have low CpG dinucleotide content as defined herein, preferably the GM-CSF transgene comprises 10 or fewer CpG dinucleotides, or is CpG dinucleotide free. Alternatively or in addition, the promoter within a plasmid of the invention may have low CpG dinucleotide content as defined herein, preferably the promoter comprises 10 or fewer CpG dinucleotides, or is CpG dinucleotide free. Preferably both the GM-CSF transgene and the promoter within a plasmid of the invention have low CpG dinucleotide content as defined herein, preferably both the GM-CSF transgene and the promoter each comprise 10 or fewer CpG dinucleotides, or are CpG dinucleotide free.

Preferably, the non-viral nucleic acid molecule (e.g. plasmid) of the invention comprise a hCEF promoter having low or no CpG dinucleotide content. By low CpG content, it is meant 20 or fewer, 15 or fewer, 10 or fewer, 5 or fewer CpG dinucleotides (e.g. 20, 19, 18, 17, 16, 15, 14, 13, 12, 11, 10, 9, 8, 7, 6, 5, 4, 3, 2, 1 or 0 CpG dinucleotides). The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO: 6. The absence of CpG dinucleotides typically further improves the performance of non-viral nucleic acid molecules (e.g. plasmids) of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The non-viral nucleic acid molecule (e.g. plasmid) of the invention may be modified to allow shut down of gene expression. Standard techniques for modifying the vector in this way are known in the art. As a non-limiting example, Tet-responsive promoters are widely used.

A non-viral nucleic acid molecule (e.g. plasmid) of the invention may be codon-optimised as described herein.

Methods of preparing plasmid DNA are well known in the art. Typically, they are capable of autonomous replication in an appropriate host or producer cell.

Host cells containing (e.g. transformed, transfected, or electroporated with) the plasmid may be prokaryotic or eukaryotic in nature, either stably or transiently transformed, transfected, or electroporated with the plasmid. Suitable host cells include bacterial, yeast, fungal, invertebrate, and mammalian cells. Preferably the host cell is bacterial; more preferably E. coli.

Host cells can then be used in methods for the large scale production of the plasmid. The cells are grown in a suitable culture medium under favourable conditions, and the desired plasmid isolated from the cells, or from the medium in which the cells are grown, by any purification technique well known to those skilled in the art; e.g. see Sambrook et al, supra.

The invention also provides host cells comprising a nucleic acid (e.g. plasmid) of the invention. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein).

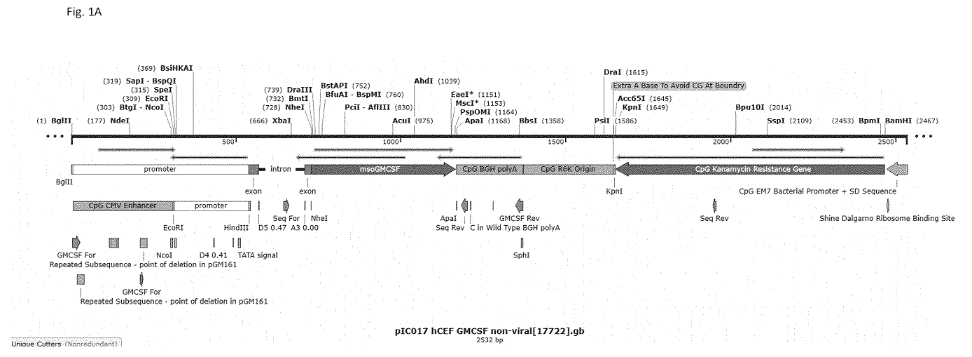

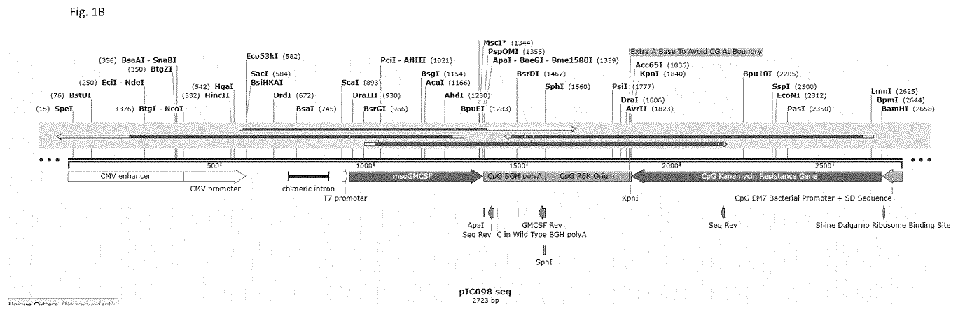

Non-limiting examples of plasmids according to the invention include pIC017 hCEF GMCSF, as illustrated in FIG. 1A and pIC098 CMV GMCSF, as illustrated in FIG. 1B.

pIC017 hCEF GMCSF (illustrated in FIG. 1A) comprises the GM-CSF transgene under the control of a hCEF promoter, a bovine growth hormone (BGM) polyA sequence, an R6K origin of replication (comprising CpG dinucleotides) and a kanamycin resistance cassette (also comprising CpG dinucleotides). Furthermore, the pIC017 plasmid further comprises a chimeric intron downstream of the enhancer/promoter region. This chimeric intron is composed of the 5′-donor site from the first intron of the human β-globin gene and the branch and 3′-acceptor site from the intron that is between the leader and the body of an immunoglobulin gene heavy chain variable region. The sequences of the donor and acceptor sites, along with the branchpoint site, have been changed to match the consensus sequences for splicing. The present of an intron, and particularly the chimeric intron in pIC017, flanking the transgene has been shown to increase the level of gene expression. An exemplary β-globin/IgG chimeric intron sequence is given in SEQ ID NO: 9. pIC017 hCEF GMCSF has the nucleic acid sequence of SEQ ID NO: 10. Variants thereof (as described therein) are also included, particularly variants with at least 90% (such as at least 90, 92, 94, 95, 96, 97, 98, 99 or 100%) to SEQ ID NO: 10. Elements of pIC017 hCEF GMCSF may be replaced to provide further exemplary plasmids of the invention. By way of non-limiting example, the murine GM-CSF transgene may be replaced by a human GM-CSF transgene, such as that of SEQ ID NO: 2 as described herein, the hCEF promoter may be replaced by another promoter, preferably an inducible promoter as described herein, and/or the CpG dinucleotides may be removed from one or more element of the pIC017 hCEF GMCSF plasmid.

pIC098 CMV GMCSF (illustrated in FIG. 1B) comprises the GM-CSF transgene under the control of a CMV promoter, a bovine growth hormone (BGM) polyA sequence, an R6K origin of replication (comprising CpG dinucleotides) and a kanamycin resistance cassette (also comprising CpG dinucleotides). pIC098 also comprises β-globin/IgG chimeric intron, as described above in the context of pIC017. pIC098 CMV GMCSF has the nucleic acid sequence of SEQ ID NO: 11. Variants thereof (as described therein) are also included, particularly variants with at least 90% (such as at least 90, 92, 94, 95, 96, 97, 98, 99 or 100%) to SEQ ID NO: 11. Elements of pIC098 CMV GMCSF may be replaced to provide further exemplary plasmids of the invention. By way of non-limiting example, the murine GM-CSF transgene may be replaced by a human GM-CSF transgene, such as that of SEQ ID NO: 2 as described herein, the CMV promoter may be replaced by another promoter, preferably an inducible promoter as described herein, and/or the CpG dinucleotides may be removed from one or more element of the pIC098 CMV GMCSF plasmid.

mRNA and saRNA

The non-viral nucleic acid molecule of the invention may be an mRNA or a self-amplifying RNA (saRNA) which encodes for GM-CSF. Both mRNA and saRNA can transfect target cells. Once inside the target cell, the mRNA or saRNA is translated by the host cell, resulting in production of the GM-CSF protein. Typically the mRNA and/or saRNA molecule is a linear RNA molecule.

In addition to the nucleic acid sequence encoding GM-CSF, an mRNA of the invention typically comprises the following basic elements: (i) a cap; (ii) a 5′ UTR; (iii) a 3′UTR; and (iv) a poly(A) tail (which may be of variable length). These elements may be as defined herein. An mRNA of the invention is typically of sequence length from about 0.2 kb to about 10 kb, such as from about 0.2 kb to about 7 kb, from about 0.2 kb to about 5 kb, from about 0.5 kb to about 5 kb, or from about 0.5 kb to about 2 kb, with a sequence length of from about 0.5 kb to about 5 kb or from about 0.5 kb to about 2 kb, being preferred.

An saRNA is a type of RNA molecule with many structural similarities to mRNA: it is a linear, single-stranded RNA molecule with elements in common with an mRNA. In particular, In addition to the nucleic acid sequence encoding GM-CSF, an saRNA of the invention typically comprises the following basic elements: (i) a cap; (ii) a 5′ untranslated region (UTR, also referred to as a conserved sequence element, CSE); (iii) alphavirus non-structural proteins 1-4 (nsP1-4) which encode the replicase as described herein; (iv) a subgenomic promoter and/or an internal ribosome entry site (IRES); (v) a 3′UTR (or CSE); and (vi) a poly(A) tail (which may be of variable length). These elements may be as defined herein. The main difference between an saRNA and an mRNA is that an saRNA is typically of greater length than an mRNA. An saRNA of the invention is typically of sequence length from about 8 kb to about 15 kb, such as from about 8 kb to about 12 kb, from about 9 kb to about 12 kb, or from about 9 kb to about 10 kb, with a sequence length of from about 9 kb to about 12 kb or from about 9 kb to about 10 kb, being preferred.

The difference in size between an saRNA and an mRNA is because an saRNA typically encodes at least one protein (e.g. 1, 2, 3 or 4 additional proteins) in addition to GM-CSF. In particular, an saRNA typically encodes at least a replicase in addition to GM-CSF. Typically an saRNA encodes four extra proteins in addition to GM-CSF. The four extra proteins encode an RNA-dependent RNA polymerase (RdRP) complex which amplifies synthetic transcripts in situ, resulting in efficient expression of GM-CSF protein within a target cell. As such, lower doses/concentrations of saRNA may be required to treat a patient compared with an equivalent mRNA (or plasmid).

The backbone sequence of an saRNA, including the genes encoding the RdRP complex are typically derived from an alphavirus, such as Venezuelan Equine Encephalitis virus (VEEV), Sindbis virus (SINV), and Semliki forest virus (SFV), preferably from a VEEV.

In an saRNA of the invention, the sequence encoding GM-CSF is downstream of the subgenomic promoter and/or IRES.

mRNA and/or saRNA according to the present invention may be synthesised as unmodified or modified mRNA. Typically, the mRNA and/or saRNA may include one or more chemical or structural modifications to abrogate mRNA interaction with toll-like receptors TLR3, TLR7, TLR8, and retinoid-inducible gene I (RIG-I) to reduce immunogenicity as well as improve stability of the mRNA. Accordingly, an mRNA or saRNA molecule of the invention is typically modified to replace any uridine bases with a chemically modified alternative, pseudouridine (ψ or ψ-UTP). The use of pseudouridine is well-known in the art. Alternatively, or additionally, any cytidine bases may be replaced with a chemically modified alternative, 5-methylcytidine (m5C), again, this is well-known in the art. Substitution of uridine by pseudouridine and/or cytidine by 5-methylcytidine typically reduces degradation of mRNA and/or saRNA by a target cell, enabling enhanced translation of the mRNA and/or saRNA molecule and increased GM-CSF protein expression. Other chemically modified bases may be used, either alone or in combination with. Non-limiting examples of such bases include m6A, 5-methyluridine (m5U), 2-thiouridine (s2U) and/or N1-methylpseudouridine (N1-m ψ-UTP), with N1-m ψ-UTP being particularly preferred.

Other modifications to an mRNA and/or saRNA may be made alternatively or in addition to chemical modification of one or more base as described above. Any combination of the modifications as described herein may be used.

mRNAs and/or saRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

mRNAs and/or saRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

Stabilising modifications may be made to either or both the 3′ and 5′ ends of the mRNA and/or saRNA. Preferably stabilising modifications are made at the 5′ end, and optionally also the 3′ end. Non-limiting examples of stabilising modifications include, e.g., end capping, polyA tail, replacement of unstable non-coding sequences (such as adenylate uridylate rich elements (AREs) or addition or 3′ or 5′ untranslated sequences from stable mRNA (such as, e.g., β-globin, actin, GAPDH, tubulin, histone, or citric acid cycle enzyme mRNA). Stabilising modifications may also be made within the mRNA and/or saRNA, and include, e.g., codon optimization and/or modification of the Kozak sequence. and/or incorporation of modified nucleosides (such as, e.g., pyrrolo-pyrimidine, C5-iodouridine, 2-amino adenosine, and 2-thiothymidine).

Typically a mRNA and/or saRNA of the invention comprises a cap. The presence of the cap is important in providing resistance to nucleases found in most eukaryotic cells. 5′ capping typically stabilises the mRNA and/or saRNA and helps the molecule evade the patient's immune system. Thus, in some embodiments, mRNAs and/or saRNAs of the invention include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. The 5′ cap is not particularly limited, and examples of 5′ caps are known in the art. By way of non-limiting example, 5′ caps include Cap1 (^{m7}GpppG_{2′Om} N), Cap2 (^{m7} GpppN_{2′Om} N_{2′Om}), m⁷ GpppG analog, anti-reverse cap analog (ARCA; m₂^{7,3′-O} GpppG), m7G(5′)ppp (5′(A,G(5′)ppp(5′)A and G(5′)ppp(5′)G. A 5′ Cap1 may be preferred, as it mimic natural eukaryotic mRNA structures, and the 2′O methylation may reduce recognition of the mRNA and/or saRNA by pattern recognition receptors.

An mRNA and/or saRNA of the invention may include a 5′ and/or 3′ untranslated region (UTR). 5′ and/or 3′ UTR, particularly 5′ UTR may include one or more elements that improve the nuclease resistance and/or improve the half-life of the mRNA and/or saRNA, for example, an iron responsive element. A 5′ UTR may be between about 50 and 500 nucleotides in length. A 3′ UTR may include one or more of a polyadenylation signal (e.g. a poly(A) tail as described herein), a binding site for protein(s) that affect the stability and/or intracellular location of the mRNA and/or saRNA, and/or one or more binding sites for miRNAs. A 3′ UTR may be between 50 and 500 nucleotides in length or longer. In an saRNA of the invention, the 5′ and/or 3′ UTR may be conserved sequence elements (CSEs). CSEs are present within alphavirus genomes, and are thought to bind to viral and/or cellular proteins and regulate viral RNA synthesis.

Typically an mRNA and/or saRNA of the invention comprises a tail. The presence of a “tail” serves to protect the mRNA and/or saRNA from exonuclease degradation, and thus increases the half-life of the mRNA and/or saRNA. Thus, an mRNA and/or saRNA of the invention may include a 3′ poly(A) tail structure. A poly-A tail on the 3′ terminus of mRNA and/or saRNA typically includes about 10 to 300 adenosine nucleotides (e.g., about 10 to 200 adenosine nucleotides, about 10 to 150 adenosine nucleotides, about 10 to 100 adenosine nucleotides, about 20 to 90 adenosine nucleotides, about 20 to 80 adenosine nucleotides or about 120 to 150 adenosine nucleotides, preferably about 80 adenosine nucleotides). In some embodiments, mRNAs of the current invention include a 3′ poly(C) tail structure. Alternatively or in addition, an mRNA and/or saRNA of the invention may include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some preferred embodiments the non-viral nucleic acid molecule of the invention is an mRNA or saRNA which comprises a sequence encoding GM-CSF and one or more of (i) uridine replaced by pseudouridine; (ii) a 5′ Cap1; and/or (ii) a poly(A) tail between about 10 to 100 adenosine nucleotides, preferably about 80 adenosine nucleotides. Particularly preferred are mRNA and/or saRNA molecules which comprise all of (i) to (iii).

An mRNA and/or saRNA of the invention will typically be a synthetic molecule that structurally resembles natural mRNA counterparts, and will rapidly express GM-CSF protein when transfected into a target cell. mRNAs and/or saRNAs according to the present invention may be synthesized according to any of a variety of known methods. For example, mRNAs and/or saRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions may be readily determined by one of skill in the art.

### Lipid Carriers

A gene therapy agent of the invention may comprise a lipid carrier to facilitate delivery to a patient and/or uptake by a target cell. Typically where a gene therapy agent of the invention comprises a non-viral nucleic acid molecule, said agent also further comprises a lipid carrier.

The lipid carrier may be formulated as a lipid nanoparticle. The phrase “lipid nanoparticle”, “lipid carrier vehicle” and “lipid-derived nanoparticle” are all used interchangeably, and refer to a delivery vehicle comprising one or more lipids (e.g., cationic lipids, non-cationic lipids, cholesterol-based lipids, and PEG-modified lipids). The contemplated lipid nanoparticles may be prepared by including multicomponent lipid mixtures of varying ratios employing one or more cationic lipids, non-cationic lipids, cholesterol-based lipids, and PEG-modified lipids. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Preferably the lipid nanoparticle is a liposome, which is a bilayer vesicle typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.).

In the context of the present invention, a lipid carrier vehicle typically serves to transport a non-viral nucleic acid molecule of the invention to a target cell. For the purposes of the present invention, the liposomal transfer vehicles are prepared to contain the desired nucleic acids. The process of incorporation of a desired entity (e.g., a non-viral nucleic acid molecule) into a liposome is often referred to as “loading” (Lasic, et al., FEBS Lett., 312: 255-258, 1992). The liposome-incorporated nucleic acids may be completely or be partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating a non-viral nucleic acid molecule of the invention into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments of the present invention, the selected transfer vehicle is capable of enhancing the stability of the non-viral nucleic acid molecule of the invention contained therein. The liposome can allow the encapsulated non-viral nucleic acid molecule of the invention to reach the target cell.

As used herein, liposomal delivery vehicles, are usually characterized as microscopic vesicles having an interior aqua space sequestered from an outer medium by a membrane of one or more bilayers.

A suitable lipid carrier may contain a cationic lipid. As used herein, the phrase “cationic lipid” refers to any of a number of lipid species that have a net positive charge at a selected pH, such as physiological pH. Several cationic lipids have been described in the literature, many of which are commercially available. In certain embodiments, the compositions of the invention may employ a lipid nanoparticles comprising an ionizable cationic lipid described in U.S. provisional patent application 61/617,468, filed Mar. 29, 2012, such as, e.g., (15Z, 18Z)—N,N-dimethyl-6-(9Z, 12Z)-octadeca-9, 12-dien-1-yl)tetracosa-15,18-dien-1-amine (HGT5000), (15Z, 18Z)—N,N-dimethyl-6-((9Z, 12Z)-octadeca-9, 12-dien-1-yl)tetracosa-4,15,18-trien-1-amine (HGT5001), and (15Z,18Z)—N,N-dimethyl-6-((9Z, 12Z)-octadeca-9, 12-dien-1-yl)tetracosa-5, 15, 18-trien-1-amine (HGT5002).

Any appropriate delivery means can be used to deliver a gene therapy agent of the invention, particularly a non-viral nucleic acid molecule (e.g. plasmid, mRNA or saRNA) of the invention to a target cell or patient. Suitable delivery means are known in the art and within the routine skill of one of ordinary skill in the art. Non-limiting examples include the use of cationic lipids, polymers (e.g. polyethyleneimine and poly-L-lysine) and electroporation.

Typically a lipid carrier according to the invention comprises one or more cationic lipid, one or more non-cationic lipid, one or more cholesterol-based lipids and one or more PEG-modified lipids.

Preferably a lipid carrier comprising one or more cationic lipids is be used to deliver a non-viral nucleic acid molecule (e.g. plasmid, mRNA or saRNA) of the invention to target cells or to a patient. Non-limiting examples of cationic lipids suitable for use according to the invention are GL67A and lipofectamine. Further non-limiting examples of lipid carriers include C12-200, HGT4003, HGT5000, HGT5001, ICE, DLinKC2-DMA, DODAP, DODMA, DLinDMA and CLinDMA, which are described in EP2858679B1, which is herein incorporated by reference in its entirety.

Non-cationic lipids that may be comprised in a lipid carrier of the invention may be defined as neutral lipids, i.e., lipids that do not carry a net charge in the conditions under which the composition is formulated and/or administered. Non-limiting examples of non-cationic lipids include DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)), and cholesterol.

Non-limiting examples of cholesterol-based lipids that may be comprised in a lipid carrier of the invention include DC-Choi (N,N-dimethyl-N-ethylcarboxamidocholesterol) and 1,4-bis(3-N-oleylamino-propyl)piperazine.

Non-limiting examples of polyethylene glycol (PEG)-modified phospholipids and derivatized lipids that may be comprised in a lipid carrier of the invention include derivatized ceramides (PEG-CER), including N-Octanoyl-Sphingosine-1-[Succinyl(Methoxy Polyethylene Glycol)-2000] (C8 PEG-2000 ceramide). PEG-modified lipids may include, but are not limited to, a polyethylene glycol chain of up to 5 kDa in length covalently attached to a lipid with alkyl chain(s) of C6-C20 length. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613).

Preferably the lipid carrier is GL67A. The cationic lipid mixture GL67A is a mixture of three components—GL67 (Cholest-5-en-3-ol (3β)-,3-[(3-aminopropyl)[4-[(3-aminopropyl)amino]butyl]carbamate], (CAS Number: 179075-30-0)), DOPE (1,2-dioleoyl-sn-glycero-3-phosphoethanolamine) and DMPE-PEG5000 (1,2-Dimyristoyl-sn-Glycero-3-Phosphoethanolamine-N-[methoxy (Polyethylene glycol)5000]). These components are formulated at a 1:2:0.05 molar ratio to form GL67A. The composition of GL67A and methods for its production are disclosed in WO2013/061091, as are methods for preparing mixtures of GL67A with exemplary non-viral vectors. The contents of WO2013/061091 are herein incorporated by reference in their entirety.

Lipofectamine consists of a 3:1 mixture of DOSPA (2,3-dioleoyloxy-N-[2(sperminecarboxamido)ethyl]-N,N-dimethyl-1-propaniminium trifluoroacetate) and DOPE.

A lipid carrier of the invention, particularly GL67A may be used in a lipid: non-viral nucleic acid molecule ratio of between about 1:1 to about 7:1, preferably of between about 2:1 to about 6:1, more preferably of between about 2:1 to about 4:1. Exemplary ratios include 1:1, 2:1, 3:1, 4:1, 5:1, 6:1 and 7:1, preferably 2:1, 3:1 or 4:1. The non-viral nucleic acid molecule may be an RNA (particularly an mRNA) or a plasmid as described herein.

### Viral Vectors

The gene therapy agent of the invention may be a viral vector. Thus, a viral vector may be used to transiently express GM-CSF within a patient to treat PAP, as described herein.

A viral vector of the invention comprises an inducible promoter as described herein. Inclusion of an inducible promoter within a viral vector of the invention allows for the concentration of GM-CSF expressed within a patient's cells to be carefully controlled, allowing transient and/or low-level expression, such that GM-CSF is expressed within a narrow toxicity/efficacy window. In this way, viral vectors of the invention allow for the treatment of PAP, particularly aPAP, whilst decreasing or eliminating side effects associated with over-expression of GM-CSF within the lungs.

A viral vector of the invention may be an retroviral vector, a lentiviral vector, an adenoviral vector, an adeno-associated viral (AAV) vector, a baculoviral vector, a herpes simplex viral (HSV) vector, or a pox viral vector. As described in detail herein, retroviral vectors and lentiviral vectors are preferred, and lentiviral vectors are particularly preferred.

The viral vectors of the present invention enable therapeutic levels of expression of GM-CSF. The viral vectors of the present invention typically provide therapeutic expression levels of GM-CSF when administered to a patient. Expression may be measured by any appropriate method (qualitative or quantitative, preferably quantitative), and concentrations given in any appropriate unit of measurement, for example ng/ml or nM. As described herein, a therapeutic level or concentration of GM-CSF expression may be comparatively low, and even potentially below the lower limit of detection using standard assays, such as quantifying GM-CSF levels in bronchoalveolar lavage fluid (BALF) or in lung tissue. However, a therapeutic effect can still be quantified based on parameters such as a BALF turbidity, surfactant protein D (SF-D) concentration in the BALF or lungs, surfactant deposition in the lungs, CT scanning and/or lung function metrics, as described herein.

Viral vectors are usually non-replicating or replication impaired vectors, which means that the viral vector cannot replicate to any significant extent in normal cells (e.g. normal human cells), as measured by conventional means—e.g. via measuring DNA synthesis and/or viral titre. Non-replicating or replication impaired vectors may have become so naturally (i.e. they have been isolated as such from nature) or artificially (e.g. by breeding in vitro or by genetic manipulation). There will generally be at least one cell-type in which the replication-impaired viral vector can be grown—for example, the pox virus vector modified vaccinia Ankara (MVA) can be grown in CEF cells. In one embodiment, the vector is selected from a human or simian adenovirus or a poxvirus vector

Typically, the viral vector is incapable of causing a significant infection in an animal subject, typically in a mammalian subject such as a human or other primate.

The nucleic acid sequence encoding GM-CSF to be included in a viral vector of the invention may be modified to facilitate expression. For example, the GM-CSF transgene sequence may be in CpG-depleted (or CpG-fee) and/or codon-optimised form to facilitate gene expression. Standard techniques for modifying the transgene sequence in this way are known in the art. The promoter within the viral vector may be CpG-depleted (or CpG-fee) and/or codon-optimised. The genome of the viral vector may have low CpG dinucleotide content, or be CpG dinucleotide free (the disclosure above in relation to codon-optimisation and/or CpG depletion in relation to non-viral nucleic acid molecules applies equally and without reservation to viral vectors of the invention).

The viral vectors of the invention may be used to transduce isolated and expanded stem/progenitor cells ex vivo prior administration to a patient. Preferably, the viral vectors of the invention are used to transduce cells within the lung (or airways/respiratory tract) in vivo.

The invention also provides host cells comprising a viral vector of the invention. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein).

### Retroviral and Lentiviral Vectors

The gene therapy agent of the invention may be a retroviral or lentiviral viral vector. Thus, a retroviral/lentiviral vector may be used to transiently express GM-CSF within a patient to treat PAP, as described herein.

Retroviral/lentiviral vectors of the invention, can integrate into the genome of transduced cells. In the context of the present invention, integration of a retroviral/lentiviral vector into the genome of a target cell has the potential to allow transient expression of GM-CSF to be induced over a long period of time, rather than continuous lost-lasting expression. This is because once integrated, the inducible promoter may be induced transiently to turn on GM-CSF expression according to a patient's clinical needs (as described here), and this induction/transient expression may be repeated according to a patient's clinical needs over a long period of time.

A retroviral/lentiviral vector of the invention comprises an inducible promoter as described herein. Inclusion of an inducible promoter within a retroviral/lentiviral vector of the invention allows for the concentration of GM-CSF expressed (particularly free GM-CSF) within a patient's cells to be carefully controlled, allowing transient and/or low-level expression, such that GM-CSF (particularly free GM-CSF) is expressed within a narrow toxicity/efficacy window. In this way, retroviral/lentiviral vectors of the invention allow for the treatment of PAP, particularly aPAP, whilst decreasing or eliminating side effects associated with over-expression of GM-CSF within the lungs. Alternatively, a promoter may be selected which provides transient GM-CSF expression. By way of non-limiting example, a CMV promoter (or CMV promoter and enhancer) has been exemplified by the present inventors to drive expression for less than 22 days. Therefore unlike for viral gene therapy agents for other indications, where prolonged expression is desired, the present invention may relate to a retroviral/lentiviral vector in which GM-CSF expression is under the control of a CMV promoter (or CMV promoter and enhancer).

The term “retrovirus” refers to any member of the Retroviridae family of RNA viruses that encode the enzyme reverse transcriptase. The term “lentivirus” refers to a family of retroviruses. Thus, all references herein to retroviral vectors of the invention apply equally and without reservation to lentiviral vectors. Further, all references herein to lentiviral vectors of the invention apply equally and without reservation to retroviral vectors.

Examples of retroviruses suitable for use in the present invention include gamma retroviruses such as murine leukaemia virus (MLV) and feline leukaemia virus (FLV). Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. Preferably the invention relates to lentiviral vectors and the production thereof. A particularly preferred lentiviral vector is an SIV vector (including all strains and subtypes), such as a SIV-AGM (originally isolated from African green monkeys, Cercopithecus aethiops). Alternatively the invention relates to HIV vectors.

The retroviral/lentiviral (e.g. SIV) vectors of the present invention are typically pseudotyped with hemagglutinin-neuraminidase (HN) and fusion (F) proteins from a respiratory paramyxovirus, or with G glycoprotein from Vesicular Stomatitis Virus (referred to as VSV-G or G-VSV). Preferably the lentiviral (e.g. SIV) vectors of the present invention are pseudotyped with HN and F from a respiratory paramyxovirus. Particularly preferably the respiratory paramyxovirus is a Sendai virus (murine parainfluenza virus type 1). The retroviral/lentiviral (e.g. SIV) vectors of the present invention may be pseudotyped with proteins from another virus, provided that the pseudotyping proteins do not negatively impact the manufactured titre of the vector (or even result in an increased titre of the vector) and/or transgene expression (or even result in increased transgene expression). Non-limiting examples of other proteins that may be used to pseudotype retroviral/lentiviral (e.g. SIV) vectors of the present invention include severe acute respiratory syndrome coronavirus 2 (SARS-CoV-2) spike protein or modified forms thereof. VSV-G and SARS-Cov2 spike protein used for pseudotyping are as those described in UK Patent Application Nos. 2118685.3 and International Application No. PCT/GB2022/050933, each of which is herein incorporated by reference in its entirety.

A retroviral/lentiviral (e.g. SIV) vector for use according to the invention may be integrase-competent (IC). Alternatively, the lentiviral (e.g. SIV) vector may be integrase-deficient (ID).

Viral vectors of the invention, particularly retroviral/lentiviral (e.g. SIV) vectors as described herein may transduce one or more cell types as described herein to achieve transient GM-CSF expression repeated over a long period of time.

The nucleic acid sequence encoding a therapeutic protein to be included in a retroviral/lentiviral (e.g. SIV) vector of the invention may be modified to facilitate expression. For example, the transgene sequence may be in CpG-depleted (or CpG-fee) and/or codon-optimised form to facilitate gene expression. Standard techniques for modifying the transgene sequence in this way are known in the art. The genome of the retroviral/lentiviral (e.g. SIV) vector may be fully or partially CpG-depleted (or CpG-fee) and/or codon-optimised.

Retroviral/lentiviral (e.g. SIV) vectors, such as those of the invention, can integrate into the genome of transduced cells and lead the potential for repeated transient expression over a long period of time, making them suitable for transduction of stem/progenitor cells. In the lung, several cell types with regenerative capacity have been identified as responsible for maintaining specific cell lineages in the conducting airways and alveoli. These include basal cells and submucosal gland duct cells in the upper airways, club cells and neuroendocrine cells in the bronchiolar airways, bronchioalveolar stem cells in the terminal bronchioles and type II pneumocytes in the alveoli. Therefore, and without being bound by theory, it is believed that said retroviral/lentiviral (e.g. SIV) vectors allow for transient GM-CSF expression over a long period of time by introducing the transgene into one or more long-lived airway epithelial cells or cell types, such as basal cells and submucosal gland duct cells in the upper airways, club cells and neuroendocrine cells in the bronchiolar airways, bronchioalveolar stem cells in the terminal bronchioles type II pneumocytes in the alveoli, submucosal acinar cells, ionocytes, and type I pneumocytes.

Accordingly, the retroviral/lentiviral (e.g. SIV) vectors of the invention may transduce one or more cells or cell lines with regenerative potential within the lung (including the airways and respiratory tract) to allow for transient GM-CSF expression over a long period of time. For example, the retroviral/lentiviral (e.g. SIV) vectors may transduce basal cells, such as those in the upper airways/respiratory tract. Basal cells have a central role in processes of epithelial maintenance and repair following injury. In addition, basal cells are widely distributed along the human respiratory epithelium, with a relative distribution ranging from 30% (larger airways) to 6% (smaller airways).

The retroviral/lentiviral (e.g. SIV) vectors of the invention may be used to transduce isolated and expanded stem/progenitor cells ex vivo prior administration to a patient. Preferably, the retroviral/lentiviral (e.g. SIV) vectors of the invention are used to transduce cells within the lung (or airways/respiratory tract) in vivo.

The retroviral/lentiviral (e.g. SIV) vectors of the invention demonstrate remarkable resistance to shear forces with only modest reduction in transduction ability when passaged through clinically-relevant delivery devices such as bronchoscopes, spray bottles and nebulisers.

The retroviral/lentiviral vectors of the present invention enable therapeutic levels of expression of GM-CSF (particularly free GM-CSF). The retroviral/lentiviral (e.g. SIV) vectors of the present invention typically provide therapeutic expression levels of GM-CSF when administered to a patient. Expression may be measured by any appropriate method (qualitative or quantitative, preferably quantitative), and concentrations given in any appropriate unit of measurement, for example ng/ml or nM. As described herein, a therapeutic level or concentration of GM-CSF expression may be comparatively low, and even potentially below the lower limit of detection using standard assays, such as quantifying GM-CSF levels in bronchoalveolar lavage fluid (BALF) or in lung tissue. However, a therapeutic effect can still be quantified based on parameters such as a BALF turbidity, surfactant protein D (SF-D) concentration in the BALF or lungs, surfactant deposition in the lungs, CT scanning and/or lung function metrics, as described herein.

Preferably, the invention relates to F/HN retroviral/lentiviral vectors comprising a promoter and a GM-CSF transgene, particularly SIV F/HN vectors.

A retroviral/lentiviral (e.g. SIV) vector of the invention may have its endogenous Rev response element (RRE) genomic element deleted and a retroviral RRE is inserted into an intron located within 100 bp 5′ of the splice acceptor's branch site of the intron. Said intron may be a chimeric intron, such as a β-globin/IgG chimeric intron as described herein. Such β-globin/IgG chimeric introns comprising a retroviral/lentiviral RRE are described in UK Patent Applicant No. 2213936.4 (although not in the context of, providing transient and/or low level GM-CSF expression to provide GM-CSF within a narrow therapeutic window, which is taught for the first time herein). UK Patent Applicant No. 2213936.4 is herein incorporated by reference in its entirety.

The viral vectors of the invention may be made using any suitable process known in the art. In particular, retroviral/lentiviral (e.g. SIV) vectors of the invention may be made using the methods disclosed in International Application No. PCT/GB2022/050524 which is herein incorporated by reference in its entirety.

The viral vectors of the invention, particularly the retroviral/lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO: 12.

The invention also provides host cells comprising a retroviral/lentiviral (e.g. SIV) vector of the invention. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein).

### Inducible Promoters

Expression of the GM-CSF transgene according to the invention may be controlled using an inducible promoter (also referred to interchangeably herein as a regulated promoter).

Inducible promoters may be used in non-viral nucleic acids of the invention, particularly plasmids as described herein. Inducible promoters are used in viral vectors of the invention.

The use of an inducible promoter allows expression of GM-CSF to be controlled within a target cell. In particular, the use of an inducible promoter allows for the duration and/or level of GM-CSF expression to be controlled. Thus, the use of an inducible promoter enables the duration of expression of GM-CSG within a patient's cells and/or the concentration of GM-CSF expressed within a patient's cells, to be carefully controlled, allowing transient and/or low-level expression, such that GM-CSF is expressed within a narrow toxicity/efficacy window as described herein. Thus, inducible promoters facilitate a treatment of PAP as described herein, can allow the concentration of GM-CSF (particularly free GM-CSF) to be maintained within the narrow therapeutic window, achieving a therapeutic effect for the patient, whilst reducing and/or eliminating histopathological changes within the patient that are normally associated with prolonged and/or high levels of GM-CSF expression.

As used herein, the term “inducible promoter” refers to a promoter that is initiates transcription only when it receives a stimulus, typically an exogenous stimulus. An inducible promoter may be regulated by an exogenous factor (also referred to as an inducer), such as a steroid, chemical inducer of dimerization, or another inducer, and may initiate transcription only when it is stimulated by said inducer.

Inducible promoters may be positive inducible, whereby in the off state, the promoter is inactive because its activator protein cannot bind. After the inducer binds to the activator protein, the activator protein can bind to the promoter, turning it on and initiating transcription.

Inducible promoters may be negative inducible, whereby in the off state, the promoter is inactive because a bound repressor protein actively prevents transcription. Once an inducer binds the repressor protein, the repressor protein is removed from the DNA. With the repressor protein absent, transcription is turned on.

Chemical agents, temperature, and light are all examples of factors that can lead to the induction of a promoter. Other non-limiting examples of inducible promoters that could be used according to the invention are synthetic promoters that rely upon endogenous transcription elements (such as those produced by Sympromics-AskBio).

Non-limiting examples of chemically-regulated/inducible promoters include steroid-regulated promoters (e.g. the AlcA promoter which is activated by AlcR) and alcohol-regulated promoters (e.g. the LexA promoter which is activated by XVE, or the GeneSwitch™ system by Thermo Fisher). Rapamycin-induced dimerization system is another example of a chemically-regulated promoter.

Non-limiting examples of temperature inducible/regulated promoters include the heat shock-inducible Hsp70 or Hsp90-derived promoters, in which a gene of choice is only expressed following exposure to a brief heat shock.

Typically the inducible promoters of the invention may be regulated by chemical agents/inducers. Chemically-regulated promoters are typically easier to induce in an in vivo/therapeutic setting. Regulated promoter systems typically comprise a regulated promoter (which can replace hCEF or any of the other promoters described herein) and a transactivator (which may be encoded by a regulatory plasmid or by a plasmid of the invention). By way of non-limiting example, in the context of a retroviral/lentiviral (e.g. SIV) vector of the invention, the vector genome plasmid (pDNA1) may comprise a GM-CSF transgene operably linked to an regulated promoter. The pDNA1 may further encodes the corresponding trans-activator. Thus, the GM-CSF transgene operably linked to the regulated promoter and the trans-activator can be encoded by a single retroviral/lentiviral (e.g. SIV) vector. In the single retroviral/lentiviral (e.g. SIV) vector system, the (i) GM-CSF transgene operably linked to the regulated promoter and (ii) the gene encoding the trans-activator are present in the same vector backbone, typically in opposite orientations. Alternatively, the GM-CSF transgene operably linked to the regulated promoter may be encoded by a first retroviral/lentiviral (e.g. SIV) vector and the trans-activator may be encoded by a second retroviral/lentiviral (e.g. SIV) vector. Preferably, a two-vector system is used, i.e. the trans-activator is encoded on a second/separate retroviral/lentiviral (e.g. SIV) vector to the GM-CSF transgene operably linked to the regulated promoter. By way of further non-limiting example, for a non-viral nucleic acid molecule of the invention, such as a plasmid for delivery to a patient, said non-viral nucleic acid molecule (e.g. plasmid) may comprise a GM-CSF transgene operably linked to an regulated promoter. The non-viral nucleic acid molecule (e.g. plasmid) may further encodes the corresponding trans-activator. Thus, the GM-CSF transgene operably linked to the regulated promoter and the trans-activator can be encoded by a single non-viral nucleic acid molecule (e.g. plasmid). In the single non-viral nucleic acid molecule (e.g. plasmid) system, the (i) GM-CSF transgene operably linked to the regulated promoter and (ii) the gene encoding the trans-activator are present in the same non-viral nucleic acid molecule (e.g. plasmid), typically in opposite orientations. Alternatively, the GM-CSF transgene operably linked to the regulated promoter may be encoded by a first non-viral nucleic acid molecule (e.g. plasmid) and the trans-activator may be encoded by a second non-viral nucleic acid molecule (e.g. plasmid). Both these non-viral nucleic acid molecules (e.g. plasmids) may be comprised in a lipid carrier as described herein for delivery to a patient.

In some embodiments a steroid-regulated promoter may be used. Steroid-regulated promoter systems are known in the art, with suitable systems being commercially available (e.g. the GeneSwitch™ system by Thermo Fisher). Use of such steroid-regulated promoters with non-viral nucleic acid molecules and viral/retroviral/lentiviral (e.g. SIV) vectors of the invention is within the routine practice of one of ordinary skill in the art.

Steroid-regulated promoter systems typically comprise a steroid-regulated promoter (which can replace hCEF or any of the other promoters described herein) and a transactivator (which may be encoded by a regulatory plasmid or by a plasmid of the invention). By way of non-limiting example, in the context of a retroviral/lentiviral (e.g. SIV) vector of the invention, the vector genome plasmid (pDNA1) may comprise a GM-CSF transgene operably linked to a steroid-regulated promoter. The pDNA1 may further encodes the corresponding trans-activator. Thus, the GM-CSF transgene operably linked to the steroid-regulated promoter and the trans-activator can be encoded by a single retroviral/lentiviral (e.g. SIV) vector. In the single retroviral/lentiviral (e.g. SIV) vector system, the (i) GM-CSF transgene operably linked to the steroid-regulated promoter and (ii) the gene encoding the trans-activator are present in the same vector backbone, typically in opposite orientations. Alternatively, the GM-CSF transgene operably linked to the steroid-regulated promoter may be encoded by a first retroviral/lentiviral (e.g. SIV) vector and the trans-activator may be encoded by a second retroviral/lentiviral (e.g. SIV) vector. Preferably, a two-vector system is used, i.e. the trans-activator is encoded on a second/separate retroviral/lentiviral (e.g. SIV) vector to the GM-CSF transgene operably linked to the steroid-regulated promoter. By way of further non-limiting example, for a non-viral nucleic acid molecule of the invention, such as a plasmid for delivery to a patient, said non-viral nucleic acid molecule (e.g. plasmid) may comprise a GM-CSF transgene operably linked to a steroid-regulated promoter. The non-viral nucleic acid molecule (e.g. plasmid) may further encodes the corresponding trans-activator. Thus, the GM-CSF transgene operably linked to the steroid-regulated promoter and the trans-activator can be encoded by a single non-viral nucleic acid molecule (e.g. plasmid). In the single non-viral nucleic acid molecule (e.g. plasmid) system, the (i) GM-CSF transgene operably linked to the steroid-regulated promoter and (ii) the gene encoding the trans-activator are present in the same non-viral nucleic acid molecule (e.g. plasmid), typically in opposite orientations. Alternatively, the GM-CSF transgene operably linked to the steroid-regulated promoter may be encoded by a first non-viral nucleic acid molecule (e.g. plasmid) and the trans-activator may be encoded by a second non-viral nucleic acid molecule (e.g. plasmid). Both these non-viral nucleic acid molecules (e.g. plasmids) may be comprised in a lipid carrier as described herein for delivery to a patient.

A trans-activator typically comprises or consists of three parts: (i) a DNA-binding domain, which is composed of zinc finger; (ii) a drug or ligand binding domain (which binds to the inducer, e.g. mifepristone); and (iii) an activation domain (e.g. p65), which is needed for turning on transgene expression. The activator will be present will be present within the gene therapy agent and hence the target cells all the time following delivery of the gene therapy agent. However, it will only be activated when the inducer is also delivered. Once the inducer is delivered the trans-activator becomes functional and will search for its specific DNA binding site, known as the zinc finger binding sequence. The GM-CSF transgene cassette under the control of an inducible promoter will comprise or consist of: (i) a zinc finger binding sequence; (ii) GM-CSF cDNA; and (iii) a bovine growth hormone polyadenylation sequence to facilitate correct processing of the GM-CSF transgene.

In a patient, GM-CSF transgene expression by target cells may be initiated by administration of the appropriate activating agent, such as the appropriate steroid when using a steroid-regulated promoter (mifepristone in the case of a mifepristone-regulated promoter, such as GeneSwitch™, or a one vector variation thereof).

One non-limiting example of a steroid-regulated promoter which may be used with the present invention is a mifepristone-regulated promoter, such as the commercially available GeneSwitch™. This exemplary mifepristone-regulated promoter has the following structure: (i) a GAL4 upstream activating sequence (UAS), which may comprise six GAL4 binding site; (ii) the adenovirus E1b TATA box; and (iii) an intron (e.g. the synthetic intron IVS8). A non-limiting example of a mifepristone-regulated promoter sequence is found in SEQ ID NO: 13. An exemplary trans-activator for use with a mifepristone-regulated promoter may have the following structure: (i) a GAL4 DNA-binding domain (DBD); (ii) a human progesterone receptor ligand binding domain (IPR-LBD) which binds to mifepristone; and (iii) human NF-κB p65 activation domain (AD). A non-limiting example of a nucleic acid sequence encoding a trans-activator for use with a mifepristone-regulated promoter is found in SEQ ID NO: 14. In this exemplary two vector system, in the presence of mifepristone, the hPR-LBD domain on the GeneSwitch™ regulatory protein undergoes a conformational change, enabling activation of the GAL4-E1b promoter, resulting in transgene expression. The trans-activator further upregulates its own expression by binding to a Gal4 DNA Binding Domain upstream of the HSV TK promoter, therefore amplifying the induction of expression of the gene of interest. In an exemplary one vector system, the regulated promoter upstream of GM-CSF is SEQ ID NO: 13, and the trans-activator of SEQ ID NO: 14 is also used. However, the promoter sequence driving expression of the in the one vector system is a constitutive promoter, such as hCEF.

### Methods of Production

Methods for the production of retroviral/lentiviral (e.g. SIV) vectors of the invention as also described herein.

The present inventors have previously demonstrated that the use of codon-optimised gal-pol genes from SIV does not negatively impact the manufactured titre of a SIV vector pseudotyped with hemagglutinin-neuraminidase (HN) and fusion (F) proteins from a respiratory paramyxovirus, and can even result in an increased titre of the vector. This is described in PCT/GB2022/050524, which is herein incorporated by reference in its entirety. Further, the inventors have shown that retroviral vectors comprising a retroviral/lentiviral RNA sequence comprising (i) codon substitutions and (ii) a reduced number of modified retroviral/lentiviral open reading frames (ORFs) do not negatively impact the manufactured vector titre, transgene expression and/or integration of the retroviral/lentiviral RNA sequence into the host/target cell genome, and can even result in an increase in vector titre, transgene expression and/or integration of the retroviral/lentiviral RNA sequence. This is described in UK Application No. 2212472.1, which is herein incorporated by reference in its entirety.

Accordingly, the present invention provides a method of producing a retroviral/lentiviral (e.g. SIV) vector comprising a GM-CSF transgene operably linked to an inducible promoter, such as an inducible promoter described herein.

A retroviral/lentiviral (e.g. SIV) may typically be pseudotyped with hemagglutinin-neuraminidase (HN) and fusion (F) proteins from a respiratory paramyxovirus or with VSV-G, and which comprises a promoter and a transgene. Preferably said retroviral/lentiviral (e.g. SIV) vector is a lentiviral vector, with Simian immunodeficiency virus (SIV) vectors being particularly preferred.

The method of the invention may be a scalable GMP-compatible method.

The method of the invention allows the generation of high titres of retroviral/lentiviral (e.g. SIV) vectors as described herein, which exhibit therapeutic levels of GM-CSF transgene expression. Typically a method of the invention produces retroviral/lentiviral (e.g. SIV) vectors as described herein that allow expression of GM-CSF to be controlled within a target cell. In particular, the retroviral/lentiviral (e.g. SIV) vectors of the invention allow for the duration and/or level of GM-CSF expression to be controlled. Thus, the inclusion of an inducible promoter within a retroviral/lentiviral (e.g. SIV) vector of the invention enables the duration of expression of GM-CSG within a patient's cells and/or the concentration of GM-CSF expressed within a patient's cells, to be carefully controlled, allowing transient and/or low-level expression, such that GM-CSF is expressed within a narrow toxicity/efficacy window as described herein.

A method of the invention typically allows the generation of retroviral/lentiviral (e.g. SIV) vectors comprising a modified retroviral/lentiviral (e.g. SIV) RNA sequence with high levels of vector integration into the host/target cell genome. Alternatively or additionally, a method of the invention may allow the generation of high titre purified retroviral/lentiviral (e.g. SIV) vectors comprising a modified retroviral/lentiviral (e.g. SIV) RNA sequence. These advantageous properties of the vectors and methods of the invention are as described in UK Application No. 2212472.1, which is herein incorporated by reference in its entirety.

The production of a two retroviral/lentiviral (e.g. SIV) vector system typically employs one or more plasmids which provide the elements needed for the production of the vector comprising the GM-CSF transgene: the genome for the retroviral/lentiviral vector, the Gag-Pol, Rev, F and HN. Multiple elements can be provided on a single plasmid. Preferably each element is provided on a separate plasmid, such that there five plasmids, one for each of the vector genome, the Gag-Pol, Rev, F and HN, respectively. Alternatively, a single plasmid may provide the Gag-Pol and Rev elements, and may be referred to as a packaging plasmid (pDNA2). The remaining elements (genome, F and HN) may be provided by separate plasmids (pDNA1, pDNA3a, pDNA3b respectively), such that four plasmids are used for the production of a retroviral/lentiviral (e.g. SIV) vector comprising the GM-CSF transgene according to the invention. In the four plasmid methods, pDNA1, pDNA3a and pDNA3b may be as described herein in the context of the five-plasmid method. In a two vector system, the trans-activator is encoded by an alternative vector genome plasmid (pDNA1*). The remaining elements may be encoded by (i) the same pDNA2a, pDNA2b, pDNA3a and pDNA3b (in a five plasmid method); or (ii) the same pDNA2, pDNA3a and pDNA3b (in a four plasmid method) as used to produce the vector comprising the GM-CSF transgene.

In a one vector system, the transgene encoding the trans-activator is encoded by the same vector genome plasmid as the GM-CSF transgene (pDNA1^{ta+}). This vector genome plasmid may be used in a four or five plasmid method to produce a retroviral/lentiviral (e.g. SIV) one vector system according to the invention. Thus, the remaining elements may be encoded by (i) the same pDNA2a, pDNA2b, pDNA3a and pDNA3b (in a five plasmid method); or (ii) the same pDNA2, pDNA3a and pDNA3b (in a four plasmid method) as used to produce the vector comprising the GM-CSF transgene in the two vector system. For retroviral/lentiviral (e.g. SIV) vectors pseudotyped with another envelope protein, such as VSV-G, rather than F and HN proteins, again a method for producing a two vector system of the invention typically employs one or more plasmids which provide the elements needed for the production of the vector comprising the GM-CSF transgene: the genome for the retroviral/lentiviral vector, the Gag-Pol (pDNA2a), Rev (pDNA2b), and envelope (e.g. VSV-G) (pDNA3). Multiple elements can be provided on a single plasmid. Preferably each element is provided on a separate plasmid, such that there are four plasmids, one for each of the vector genome, the Gag-Pol, Rev and envelope (e.g. VSV-G), respectively. In the four plasmid methods for producing VSV-G pseudotyped retroviral/lentiviral vectors comprising the GM-CSF transgene for two vector systems, pDNA1, pDNA2a and pDNA2b may be as described herein in the context of the five-plasmid method for producing two retroviral/lentiviral vector systems pseudotyped with F and HN proteins. Alternatively, a single plasmid may provide the Gag-Pol and Rev elements, and may be referred to as a packaging plasmid (pDNA2). The remaining elements (genome and VSV-G) may be provided by separate plasmids (pDNA1 and pDNA3 respectively), such that three plasmids are used for the production of a retroviral/lentiviral (e.g. SIV) vectors comprising the GM-CSF transgene for two vector system according to the invention. In the three plasmid methods, pDNA1 may be as described herein in the context of the five/four-plasmid methods. In a two vector system, the trans-activator is encoded by an alternative vector genome plasmid (pDNA1*). The remaining elements may be encoded by (i) the same pDNA2a, pDNA2b and pDNA3 (in a four plasmid method); or (ii) the same pDNA2 and pDNA3 (in a three plasmid method) as used to produce the vector comprising the GM-CSF transgene.

In a one vector system, the transgene encoding the trans-activator is encoded by the same vector genome plasmid as the GM-CSF transgene (pDNA1^{ta+}). This vector genome plasmid may be used in a four or three plasmid method to produce a retroviral/lentiviral (e.g. SIV) one vector system according to the invention. Thus, the remaining elements may be encoded by (i) the same pDNA2a, pDNA2b and pDNA3 (in a four plasmid method); or (ii) the same pDNA2 and pDNA3 (in a three plasmid method) as used to produce the vector comprising the GM-CSF transgene in the two vector system.

Preferably, the vector genome plasmid encodes all the genetic material that is packaged into the final retroviral/lentiviral vector, including the transgene. The vector genome plasmid may be designated herein as “pDNA1”, and typically comprises the GM-CSF transgene. In a two vector system, the trans-activator is encoded by an alternative vector genome plasmid (pDNA1*). In a one vector system, the transgene encoding the trans-activator is encoded by the same vector genome plasmid as the GM-CSF transgene (pDNA1*). In a preferred five plasmid method for producing the retroviral/lentiviral vector comprising the GM-CSF transgene, the other plasmids are manufacturing plasmids encoding the Gag-Pol, Rev, F and HN proteins. These plasmids may be designated “pDNA2a”, “pDNA2b”, “pDNA3a” and “pDNA3b” respectively.

Typically, the lentivirus is SIV, such as SIV1, preferably SIV-AGM. The F and HN proteins are derived from a respiratory paramyxovirus, preferably a Sendai virus.

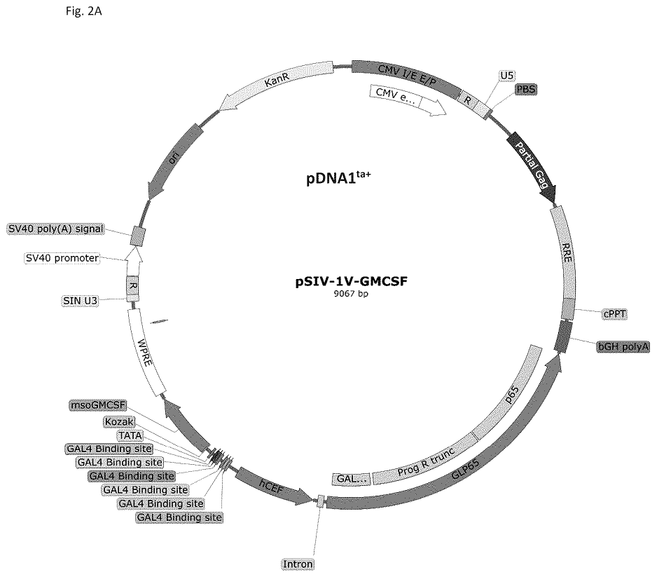

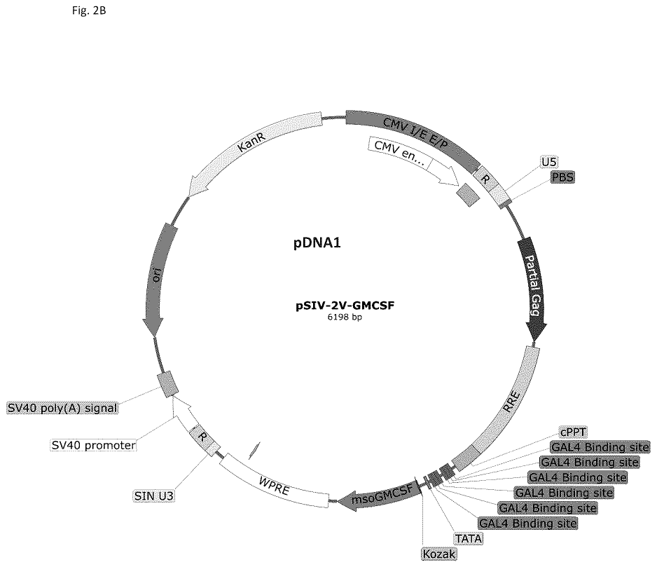

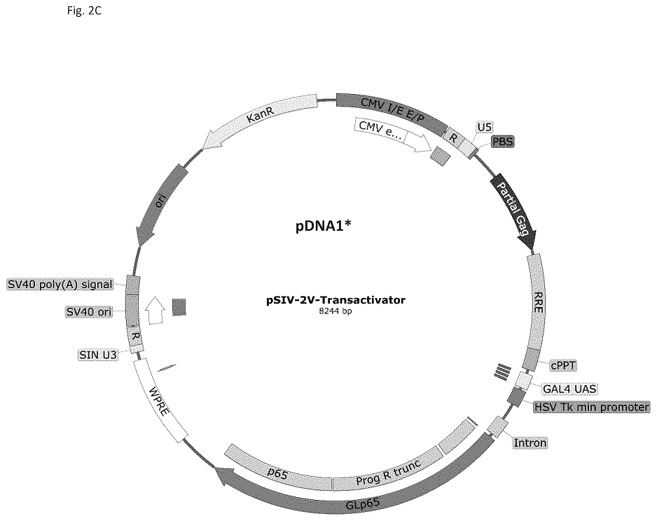

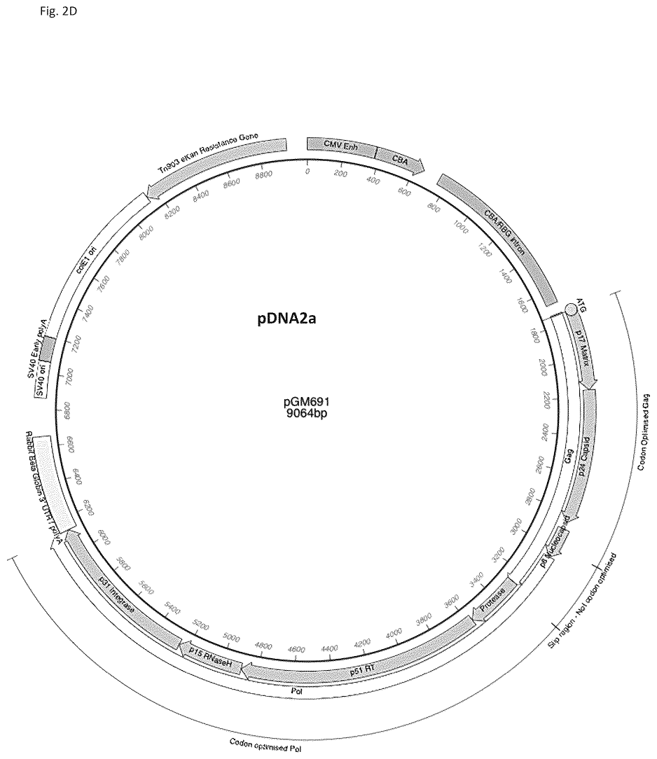

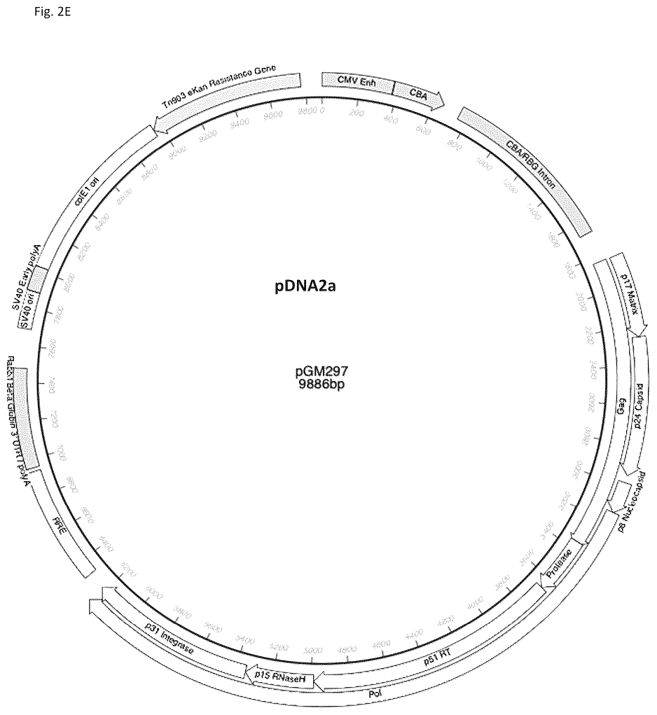

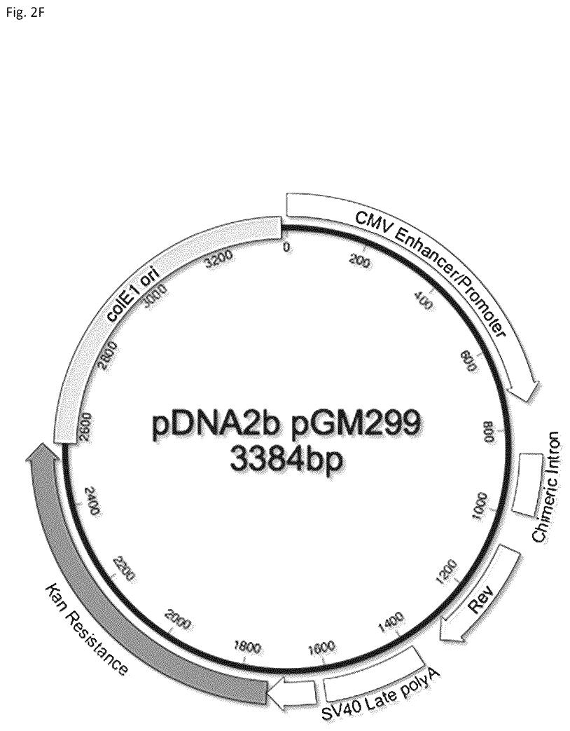

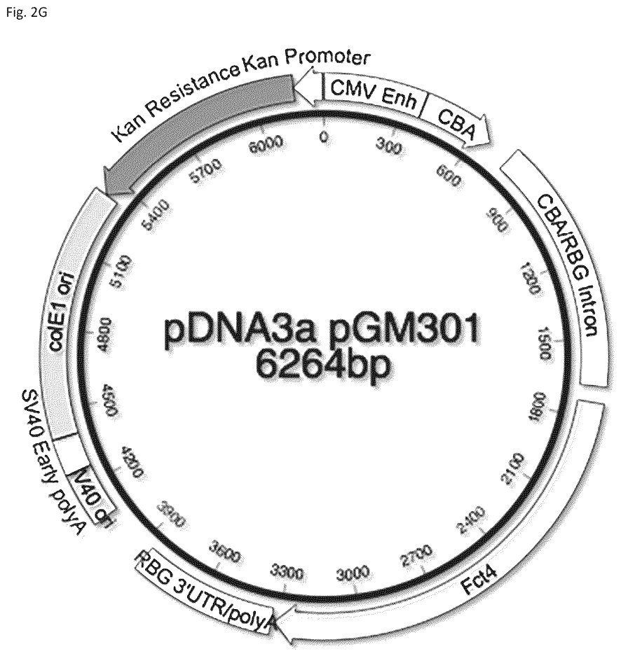

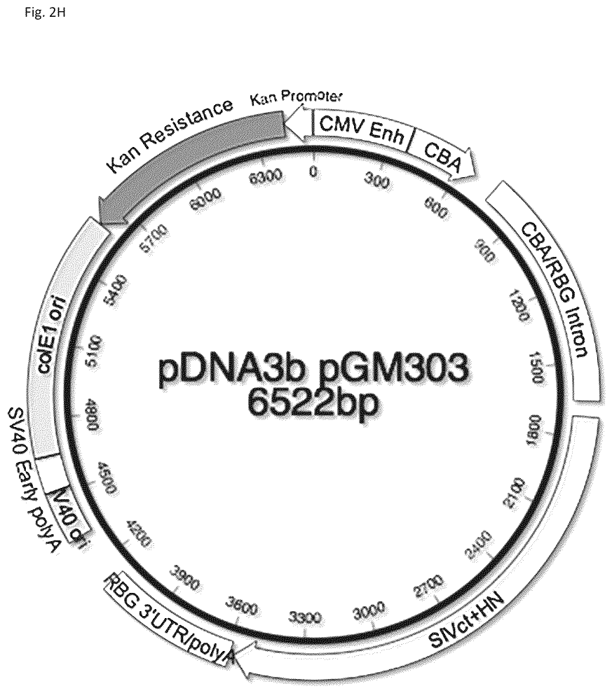

In a specific embodiment the five plasmids for producing an SIV vector comprising a GM-CSF transgene as part of a two vector system are characterised by FIGS. 2B and 2D-H, thus pDNA1 is the pSIV-2V-GMCSF plasmid of FIG. 2B, pDNA2a is the pGM691 plasmid of FIG. 2D or the pGM297 plasmid of FIG. 2E, pDNA2b is the pGM299 plasmid of FIG. 2F, pDNA3a is the pGM301 plasmid of FIG. 2G and pDNA3b is the pGM303 plasmid of FIG. 2H, or variants thereof any of these plasmids (as described herein).

The plasmid as defined in FIG. 2B is represented by SEQ ID NO: 15; the plasmid as defined in FIG. 2D is represented by SEQ ID NO: 16; the plasmid as defined in FIG. 2E is represented by SEQ ID NO: 17; the plasmid as defined in FIG. 2F is represented by SEQ ID NO: 18; the plasmid as defined in FIG. 2G is represented by SEQ ID NO: 19; the plasmid as defined in FIG. 2H is represented by SEQ ID NO: 20. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 15 to 20 are encompassed.

In a specific embodiment the five plasmids for producing an SIV vector comprising a transactivator as part of a two vector system are characterised by FIGS. 2C-2H, thus pDNA1* is the pSIV-2V-Transactivator plasmid of FIG. 2C, pDNA2a is the pGM691 plasmid of FIG. 2D or the pGM297 plasmid of FIG. 2E, pDNA2b is the pGM299 plasmid of FIG. 2F, pDNA3a is the pGM301 plasmid of FIG. 2G and pDNA3b is the pGM303 plasmid of FIG. 2H, or variants thereof any of these plasmids (as described herein).

The plasmid as defined in FIG. 2C is represented by SEQ ID NO: 21; the plasmid as defined in FIG. 2D is represented by SEQ ID NO: 16; the plasmid as defined in FIG. 2E is represented by SEQ ID NO: 17; the plasmid as defined in FIG. 2F is represented by SEQ ID NO: 18; the plasmid as defined in FIG. 2G is represented by SEQ ID NO: 19; the plasmid as defined in FIG. 2H is represented by SEQ ID NO: 20. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 16 to 21 are encompassed.

In a specific embodiment the five plasmids for producing an SIV vector comprising a GM-CSF transgene and transactivator as part of a one vector system are characterised by FIGS. 2A and 2D-2H, thus pDNA1^{ta+} is the pSIV-1V-GM-CSF plasmid of FIG. 2A, pDNA2a is the pGM691 plasmid of FIG. 2D or the pGM297 plasmid of FIG. 2E, pDNA2b is the pGM299 plasmid of FIG. 2F, pDNA3a is the pGM301 plasmid of FIG. 2G and pDNA3b is the pGM303 plasmid of FIG. 2H, or variants thereof any of these plasmids (as described herein).

The plasmid as defined in FIG. 2A is represented by SEQ ID NO: 22; the plasmid as defined in FIG. 2D is represented by SEQ ID NO: 16; the plasmid as defined in FIG. 2E is represented by SEQ ID NO: 17; the plasmid as defined in FIG. 2F is represented by SEQ ID NO: 18; the plasmid as defined in FIG. 2G is represented by SEQ ID NO: 19; the plasmid as defined in FIG. 2H is represented by SEQ ID NO: 20. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 16 to 20 and 22 are encompassed.

In each of the three-, four- or five-plasmid methods of the invention all of the plasmids contribute to the formation of the final retroviral/lentiviral (e.g. SIV) vector (whether encoding the GM-CSF transgene or the trans-activator for a two vector system, or both the GM-CSF transgene and the trans-activator in a one vector system), although only the vector genome plasmid provides nucleic acid sequence comprised in the retroviral/lentiviral (e.g. SIV) RNA sequence. During manufacture of the retroviral/lentiviral (e.g. SIV) vector, the vector genome plasmid (pDNA1/pDNA1*/pDNA^{ta+}) provides the enhancer/promoter, Psi, RRE-comprising intron, cPPT, mWPRE, SIN LTR, SV40 polyA (see FIG. 2A-C), which are important for virus manufacture. Using pSIV-2V-GMCSF as a non-limiting example of a pDNA1, the CMV enhancer/promoter, SV40 polyA, colE1 Ori and KanR are involved in manufacture of the retroviral/lentiviral (e.g. SIV) vector of the invention, but are not found in the final retroviral/lentiviral (e.g. SIV) vector. The cPPT (central polypurine tract), RRE-comprising intron (inserted between hCEF and the AAT transgene), hCEF, AAT (transgene) and mWPRE from pSIV-2V-GMCSF are found in the final retroviral/lentiviral (e.g. SIV) vector. SIN LTR (long terminal repeats, SIN/IN self-inactivating) and Psi (packaging signal) may be found in the final retroviral/lentiviral (e.g. SIV) vector.

For other retroviral/lentiviral (e.g. SIV) vectors of the invention, corresponding elements from the other vector genome plasmids (pDNA1) are required for manufacture (but not found in the final vector), or are present in the final retroviral/lentiviral (e.g. SIV) vector.

In a specific embodiment relating to pseudotyping with VSV-G, the four plasmids for producing an SIV vector comprising a GM-CSF transgene as part of a two vector system are characterised by FIGS. 2B, 2D-2F and 2I, thus pDNA1 is the pSIV-2V-GMCSF plasmid of FIG. 2B, pDNA2a is the pGM691 plasmid of FIG. 2D or the pGM297 plasmid of FIG. 2E, pDNA2b is the pGM299 plasmid of FIG. 2F, pDNA3 is the pMD2.G plasmid of FIG. 2I, or variants thereof any of these plasmids (as described herein). The plasmid as defined in FIG. 2B is represented by SEQ ID NO: 15; the plasmid as defined in FIG. 2D is represented by SEQ ID NO: 16; the plasmid as defined in FIG. 2E is represented by SEQ ID NO: 17; the plasmid as defined in FIG. 2F is represented by SEQ ID NO: 18; the plasmid as defined in FIG. 2I is represented by SEQ ID NO: 23. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 15 to 18 and 23 are encompassed.

In a specific embodiment the four plasmids for producing an SIV vector comprising a transactivator as part of a two vector system are characterised by FIGS. 2C-2F and 2I, thus pDNA1* is the pSIV-2V-Transactivator plasmid of FIG. 2C, pDNA2a is the pGM691 plasmid of FIG. 2D or the pGM297 plasmid of FIG. 2E, pDNA2b is the pGM299 plasmid of FIG. 2F, pDNA3 is the pMD2.G plasmid of FIG. 2I, or variants thereof any of these plasmids (as described herein).

The plasmid as defined in FIG. 2C is represented by SEQ ID NO: 21; the plasmid as defined in FIG. 2D is represented by SEQ ID NO: 16; the plasmid as defined in FIG. 2E is represented by SEQ ID NO: 17; the plasmid as defined in FIG. 2F is represented by SEQ ID NO: 18; the plasmid as defined in FIG. 2I is represented by SEQ ID NO: 23. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 16 to 18, 21 and 23 are encompassed.

In a specific embodiment the four plasmids for producing an SIV vector comprising a GM-CSF transgene and transactivator as part of a one vector system are characterised by FIGS. 2A, 2D-2F and 2I, thus pDNA1^{ta+} is the pSIV-1V-GM-CSF plasmid of FIG. 2A, pDNA2a is the pGM691 plasmid of FIG. 2D or the pGM297 plasmid of FIG. 2E, pDNA2b is the pGM299 plasmid of FIG. 2F, pDNA3 is the pMD2.G plasmid of FIG. 2I, or variants thereof any of these plasmids (as described herein).

The plasmid as defined in FIG. 2A is represented by SEQ ID NO: 22; the plasmid as defined in FIG. 2D is represented by SEQ ID NO: 16; the plasmid as defined in FIG. 2E is represented by SEQ ID NO: 17; the plasmid as defined in FIG. 2F is represented by SEQ ID NO: 18; the plasmid as defined in FIG. 2I is represented by SEQ ID NO: 23. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs: 16 to 18, 22 and 23 are encompassed.

The F and HN proteins from pDNA3a and pDNA3b (preferably Sendai F and HN proteins) or the VSV-G from pDNA3 are important for infection of target cells with the final retroviral/lentiviral (e.g. SIV) vector, i.e. for entry of a patient's epithelial cells (typically lung or nasal cells as described herein). The products of the pDNA2a and pDNA2b plasmids (or pDNA2 if the Gag-Pol and Rev elements are combined in a single plasmid) are important for virus transduction, i.e. for inserting the retroviral/lentiviral (e.g. SIV) DNA into the host's genome. The promoter, regulatory elements (such as WPRE) and transgene are important for transgene expression within the target cell(s).

A method of the invention may comprise or consist of the following steps: (a) growing cells in suspension; (b) transfecting the cells with one or more plasmids; (c) adding a nuclease; (d) harvesting the retrovirus/lentivirus (e.g. SIV); (e) adding trypsin; and (f) purification of the retrovirus/lentivirus (e.g. SIV).

This method may use the three-, four- or five-plasmid system described herein. Thus, for a five-plasmid method for producing vector comprising a GM-CSF transgene as part of a two vector system, the one or more plasmids may comprise or consist of: a vector genome plasmid pDNA1; a Gag-Pol plasmid (e.g. codon-optimised Gag-Pol plasmid), pDNA2a; a Rev plasmid, pDNA2b; a fusion (F) protein plasmid, pDNA3a; and a hemagglutinin-neuraminidase (HN) plasmid, pDNA3b. The pDNA1 may be pSIV-2V-GMCSF. The pDNA2a may be pGM297 or pGM691, preferably pGM691. The pDNA2b may be pGM299. The pDNA3a may be pGM301. The pDNA3b may be pGM303. Any combination of pDNA1, pDNA2a, pDNA2b, pDNA3a and pDNA3b may be used. Preferably, the pDNA1 is pSIV-2V-GMCSF; the pDNA2a is pGM691; the pDNA2b is pGM299; the pDNA3a is pGM301; and the pDNA3b is pGM303.

For a five-plasmid method for producing vector comprising a transactivator as part of a two vector system, the one or more plasmids may comprise or consist of: a vector genome plasmid pDNA1*; a Gag-Pol plasmid (e.g. codon-optimised Gag-Pol plasmid), pDNA2a; a Rev plasmid, pDNA2b; a fusion (F) protein plasmid, pDNA3a; and a hemagglutinin-neuraminidase (HN) plasmid, pDNA3b. The pDNA1* may be pSIV-2V-Transactivator. The pDNA2a may be pGM297 or pGM691, preferably pGM691. The pDNA2b may be pGM299. The pDNA3a may be pGM301. The pDNA3b may be pGM303. Any combination of pDNA1*, pDNA2a, pDNA2b, pDNA3a and pDNA3b may be used. Preferably, the pDNA1* is pSIV-2V-Transactivator; the pDNA2a is pGM691; the pDNA2b is pGM299; the pDNA3a is pGM301; and the pDNA3b is pGM303.

For a five-plasmid method for producing vector comprising a transactivator as part of a one vector system, the one or more plasmids may comprise or consist of: a vector genome plasmid pDNA1^{ta+}; a Gag-Pol plasmid (e.g. codon-optimised Gag-Pol plasmid), pDNA2a; a Rev plasmid, pDNA2b; a fusion (F) protein plasmid, pDNA3a; and a hemagglutinin-neuraminidase (HN) plasmid, pDNA3b. The pDNA1^{ta+} may be pSIV-1V-GMCSF. The pDNA2a may be pGM297 or pGM691, preferably pGM691. The pDNA2b may be pGM299. The pDNA3a may be pGM301. The pDNA3b may be pGM303. Any combination of pDNA1^{ta+}, pDNA2a, pDNA2b, pDNA3a and pDNA3b may be used. Preferably, the pDNA1^{ta+} is pSIV-1V-GMCSF; the pDNA2a is pGM691; the pDNA2b is pGM299; the pDNA3a is pGM301; and the pDNA3b is pGM303.

For a four-plasmid method for producing vector comprising a GM-CSF transgene as part of a two vector system, the one or more plasmids may comprise or consist of: a vector genome plasmid pDNA1; a Gag-Pol plasmid (e.g. codon-optimised Gag-Pol plasmid), pDNA2a; a Rev plasmid, pDNA2b; and a VSV-G plasmid, pDNA3. The pDNA1 may be pSIV-2V-GMCSF. The pDNA2a may be pGM297 or pGM691, preferably pGM691. The pDNA2b may be pGM299. The pDNA3 may be pMD2.G. Any combination of pDNA1, pDNA2a, pDNA2b, and pDNA3 may be used. Preferably, the pDNA1 is pSIV-2V-GMCSF; the pDNA2a is pGM691; the pDNA2b is pGM299; the pDNA3a is pGM301; and the pDNA3 is pMD2.G.

For a four-plasmid method for producing vector comprising a transactivator as part of a two vector system, the one or more plasmids may comprise or consist of: a vector genome plasmid pDNA1*; a Gag-Pol plasmid (e.g. codon-optimised Gag-Pol plasmid), pDNA2a; a Rev plasmid, pDNA2b; and a VSV-G plasmid, pDNA3. The pDNA1* may be pSIV-2V-Transactivator. The pDNA2a may be pGM297 or pGM691, preferably pGM691. The pDNA2b may be pGM299. The pDNA3 may be pMD2.G. Any combination of pDNA1*, pDNA2a, pDNA2b and pDNA3 may be used. Preferably, the pDNA1* is pSIV-2V-Transactivator; the pDNA2a is pGM691; the pDNA2b is pGM299; and the pDNA3 is pMD2.G.

For a four-plasmid method for producing vector comprising a transactivator as part of a one vector system, the one or more plasmids may comprise or consist of: a vector genome plasmid pDNA1^{ta+}; a Gag-Pol plasmid (e.g. codon-optimised Gag-Pol plasmid), pDNA2a; a Rev plasmid, pDNA2b; and a VSV-G plasmid, pDNA3. The pDNA1^{ta+} may be pSIV-1V-GMCSF. The pDNA2a may be pGM297 or pGM691, preferably pGM691. The pDNA2b may be pGM299. The pDNA3 may be pMD2.G. Any combination of pDNA1^{ta+}, pDNA2a, pDNA2b and pDNA3 may be used. Preferably, the pDNA1^{ta+} is pSIV-1V-GMCSF; the pDNA2a is pGM691; the pDNA2b is pGM299; and the pDNA3 is pMD2.G.

As described herein, a regulated promoter system, such as a steroid-regulated promoter system, typically comprise a regulated promoter and a transactivator. Preferably the vector genome plasmid (pDNA1* for a two vector system or pDNA^{ta+} in a two vector system) comprises a GM-CSF transgene operably linked to a regulated promoter, as exemplified in pSIV-2V-GMCSF (FIG. 2B and SEQ ID NO: 15) and pSIV-1V-GMCSF (FIG. 2A and SEQ ID NO: 23). The pDNA1, i.e. pDNA1^{ta+}, may further encodes the corresponding trans-activator, as exemplified in pSIV-1V-GMCSF (FIG. 2A and SEQ ID NO: 23). Thus, the transgene operably linked to the regulated promoter and the trans-activator can be encoded by a single lentiviral (e.g. SIV) vector, which may be produced according to a method of the invention. In the single vector system, the (i) GM-CSF transgene operably linked to the regulated promoter and (ii) the gene encoding the trans-activator are present in the same vector backbone, typically in opposite orientations. Alternatively, the transgene operably linked to the regulated promoter may be encoded by a first lentiviral (e.g. SIV) vector and the trans-activator may be encoded by a second lentiviral (e.g. SIV) vector.

Any appropriate ratio of vector genome plasmid:Gag-Pol plasmid:Rev plasmid:F plasmid:HN plasmid may be used to further optimise (increase) the retroviral/lentiviral (e.g. SIV) titre produced. By way of non-limiting example, the ratio of vector genome plasmid:Gag-Pol plasmid:Rev plasmid:F plasmid:HN plasmid may by in the range of 10-40:−4-20:3-12:3-12:3-12, typically 15-20:7-11:4-8:4-8:4-8, such as about 18-22:7-11:4-8:4-8:4-8, 19-21:8-10:5-7:5-7:5-7. Preferably the ratio of vector genome plasmid:Gag-Pol plasmid:Rev plasmid:F plasmid:HN plasmid is about 20:9:6:6:6. Preferably the ratio of vector genome plasmid:Gag-Pol plasmid:Rev plasmid:VSV-G plasmid is about 20:9:6:12.

Steps (a)-(f) of the method are typically carried out sequentially, starting at step (a) and continuing through to step (f). The method may include one or more additional step, such as additional purification steps, buffer exchange, concentration of the retroviral/lentiviral (e.g. SIV) vector after purification, and/or formulation of the retroviral/lentiviral (e.g. SIV) vector after purification (or concentration). Each of the steps may comprise one or more sub-steps. For example, harvesting may involve one or more steps or sub-steps, and/or purification may involve one or more steps or sub-steps.

Any appropriate cell type may be transfected with the one or more plasmids (e.g. the five-, four- or three-plasmids described herein) to produce a retroviral/lentiviral (e.g. SIV) vector of the invention. Typically mammalian cells, particularly human cell lines are used. Non-limiting examples of cells suitable for use in the methods of the invention are HEK293 cells (such as HEK293F or HEK293T cells) and 293T/17 cells. Commercial cell lines suitable for the production of virus are also readily available (e.g. Gibco Viral Production Cells—Catalogue Number A35347 from ThermoFisher Scientific).

The cells may be grown in animal-component free media, including serum-free media. The cells may be grown in a media which contains human components. The cells may be grown in a defined media comprising or consisting of synthetically produced components.

Any appropriate transfection means may be used according to the invention. Selection of appropriate transfection means is within the routine practice of one of ordinary skill in the art. By way of non-limiting example, transfection may be carried out by the use of PEIPro™, Lipofectamine2000™ or Lipofectamine3000™.

Any appropriate nuclease may be used according to the invention. Selection of appropriate nuclease is within the routine practice of one of ordinary skill in the art. Typically the nuclease is an endonuclease. By way of non-limiting example, the nuclease may be Benzonase® or Denarase®. The addition of the nuclease may be at the pre-harvest stage or at the post-harvest stage, or between harvesting steps.

The gag-pol genes used in the production of a retroviral/lentiviral (e.g. SIV) vectors of the invention may be codon-optimised. Thus, the gag-pol genes within the pDNA2a plasmid may be codon-optimised. By way of non-limiting example, codon-optimised gag-pol genes may comprise or consist of the nucleic acid sequence of SEQ ID NO: 24, or a variant thereof (as defined herein). In particular, the codon-optimised gag-pol genes of the invention may comprise or consist of a nucleic acid sequence having at least 80%, at least 85%, at least 90%, at least 95%, at least 96%, at least 97%, at least 98%, at least 99% or more sequence identity to SEQ ID NO: 24, preferably at least 95%, identity to SEQ ID NO: 24. The codon-optimised gag-po genes may consist of the nucleic acid sequence of SEQ ID NO: 24. The preferred pDNA2a, pGM691, comprises the codon-optimised gag-pol genes of SEQ ID NO: 24.

The gag-pol genes (e.g. SIV gag-pol genes), including codon-optimised gag-pol genes are typically operably linked to a promoter to facilitate expression of the gag-pol proteins. Any suitable promoter may be used, including those described herein in the context of promoters for the transgene. Preferably, the promoter is a CAG promoter, as used on the exemplified pGM691 plasmid. An exemplary CAG promoter is set out in SEQ ID NO: 25. The codon-optimised gag-pol genes of SEQ ID NO: 24 comprise a translational slip, and so do not form a single conventional open reading frame.

Codon-optimised gag-pol genes (or nucleic acids comprising or consisting thereof) and plasmids comprising said genes or nucleic acids are advantageous in the production of retroviral/lentiviral (e.g. SIV) vectors using methods of the invention, as they allow for the production of high titre retroviral/lentiviral (e.g. SIV) vectors. Typically said codon-optimised gag-pol genes (or nucleic acids comprising or consisting thereof) and plasmids comprising said genes or nucleic acids can be used to produces a titre of retroviral/lentiviral (e.g. SIV) vector that is at least equivalent to the titre of retroviral/lentiviral (e.g. SIV) vector produced by a corresponding method which does not use codon-optimised gag-pol genes, as described herein. Codon-optimised gag-pol genes are further disclosed in PCT/GB2022/050524, which is herein incorporated by reference in its entirety.

The invention also provides a retroviral/lentiviral (e.g. SIV) vector obtainable by a method of the invention.

Typically, the retroviral/lentiviral (e.g. SIV) vector obtainable by a method of the invention is produced at a high-titre, as described herein. Titre may be measured in terms of transducing units, as defined here.

Accordingly, the retroviral/lentiviral (e.g. SIV) vectors of the invention, including those obtainable by a method of the invention may optionally be at a titre of at least about 2.5×10⁶ TU/mL, at least about 3.0×10⁶ TU/mL, at least about 3.1×10⁶ TU/mL, at least about 3.2×10⁶ TU/mL, at least about 3.3×10⁶ TU/mL, at least about 3.4×10⁶ TU/mL, at least about 3.5×10⁶ TU/mL, at least about 3.6×10⁶ TU/mL, at least about 3.7×10⁶ TU/mL, at least about 3.8×10⁶ TU/mL, at least about 3.9×10⁶ TU/mL, at least about 4.0×10⁶ TU/mL or more. Preferably the retroviral/lentiviral (e.g. SIV) vector is produced at a titre of at least about 3.0×10⁶ TU/mL, or at least about 3.5×10⁶ TU/mL.

The production of high-titre retroviral/lentiviral (e.g. SIV) vectors may impart other desirable properties on the resulting vector products. For example, without being bound by theory, it is believed that production at high titres without the need for intense concentration by methods such as TFF results in a higher quality vector product than retroviral/lentiviral (e.g. SIV) vectors produced by corresponding methods without the use of codon-optimised gag-pol genes (and optionally a modified vector genome plasmid), because the vectors are exposed to less shear forces which can damage the viral particles and their RNA cargo.

Typically the gag-pol genes (e.g. codon-optimised gag-pol genes) used are matched to the retroviral/lentiviral vector being produced. By way of non-limiting example, when the lentiviral vector is an HIV vector, the codon-optimised gag-pol genes used are HIV gag-pol genes. By way of non-limiting example, when the lentiviral vector is an SIV vector, the codon-optimised gag-pol genes used are SIV gag-pol genes.

Preferably the codon-optimised gag-pol genes used are SIV gag-pol genes.

As used herein, the term “trypsin” refers to both trypsin and equivalents thereof. An equivalent enzyme is one with the same or essentially the same cleavage specificity as trypsin. Trypsin cleavage activity may be defined as cleavage C-terminal to arginine or lysine residues, typically exclusively C-terminal to arginine or lysine residues. The trypsin activity may preferably be provided by an animal origin free, recombinant enzyme such as TrypLE Select™. The addition of trypsin may be at the pre-harvest stage or at the post-harvest stage, or between harvesting steps.

Any appropriate purification means may be used to purify the retroviral/lentiviral (e.g. SIV) vector. Non-limiting examples of suitable purification steps include depth/end filtration, tangential flow filtration (TFF) and chromatography. The purification step typically comprises at least on chromatography step. Non-limiting examples of chromatography steps that may be used in accordance with the invention include mixed-mode size exclusion chromatography (SEC) and/or anion exchange chromatography. Elution may be carried out with or without the use of a salt gradient, preferably without.

### Therapeutic Indications

Viral/retroviral/lentiviral (e.g. SIV) vectors and non-viral nucleic acid molecules (e.g. plasmids, mRNAs or saRNAs) of the invention can effectively treat PAP by providing a GM-CSF transgene for the correction of the disease. Accordingly, viral/retroviral/lentiviral (e.g. SIV) vectors and non-viral nucleic acid molecules (e.g. plasmids, mRNAs or saRNAs) of the invention may be used to treat PAP (particularly aPAP), typically by gene therapy with a GM-CSF transgene as described herein. Thus, viral/retroviral/lentiviral (e.g. SIV) vectors and non-viral nucleic acid molecules (e.g. plasmids, mRNAs or saRNAs) of the invention may be considered gene therapy agents, particularly GM-CSF gene therapy agents, or may be comprised within gene therapy agents, particularly GM-CSF gene therapy agents.

Accordingly, the invention provides a method of treating PAP, particularly aPAP, the method comprising administering a viral/retroviral/lentiviral (e.g. SIV) vector and/or non-viral nucleic acid molecule (e.g. plasmid, mRNA or saRNA) of the invention to a patient in need thereof. The viral/retroviral/lentiviral (e.g. SIV) vector and/or non-viral nucleic acid molecule (e.g. plasmid, mRNA or saRNA) of the invention is typically administered to the patient at a therapeutically effective amount, which may be readily determined by a clinician without undue burden. Typically the viral/retroviral/lentiviral (e.g. SIV) vector and/or non-viral nucleic acid molecule (e.g. plasmid, mRNA or saRNA) of the invention is produced using a method of the present invention.

The invention also provides a viral/retroviral/lentiviral (e.g. SIV) vector and/or non-viral nucleic acid molecule (e.g. plasmid, mRNA or saRNA) of the invention for use in a method of treating a disease, specifically PAP, preferably aPAP. Typically the viral/retroviral/lentiviral (e.g. SIV) vector and/or non-viral nucleic acid molecule (e.g. plasmid, mRNA or saRNA) of the invention is produced using a method of the present disclosure.

The invention also provides the use of a viral/retroviral/lentiviral (e.g. SIV) vector and/or non-viral nucleic acid molecule (e.g. plasmid, mRNA or saRNA) of the invention in the manufacture of a medicament for use in a method of treating a disease, specifically PAP, preferably aPAP. Typically the viral/retroviral/lentiviral (e.g. SIV) vector and/or non-viral nucleic acid molecule (e.g. plasmid, mRNA or saRNA) of the invention is produced using a method of the present disclosure.

### Formulation, Compositions and Administration

The viral/retroviral/lentiviral (e.g. SIV) vector and/or non-viral nucleic acid molecule (e.g. plasmid, mRNA or saRNA) of the invention may be administered in any dosage appropriate for achieving the desired therapeutic effect. As described herein, typically the dose of viral/retroviral/lentiviral (e.g. SIV) vector and/or non-viral nucleic acid molecule (e.g. plasmid, mRNA or saRNA) of the invention administered is lower than that which would be used for other transgenes (e.g. CFTR or AAT), because of the narrow therapeutic window of GM-CSF. Appropriate dosages may be determined by a clinician or other medical practitioner using standard techniques and within the normal course of their work. Accordingly, non-limiting examples of suitable dosages of a viral/retroviral/lentiviral (e.g. SIV) vector include 1×10⁷ transduction units (TU), 1×10⁸ TU, 1×10⁹ TU, such as any dose in the range of 1×10⁷ TU to 1×10⁸ TU.

The invention also provides compositions comprising a viral/retroviral/lentiviral (e.g. SIV) vector and/or non-viral nucleic acid molecule (e.g. plasmid, mRNA or saRNA) of the invention, and a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long-term storage.

The viral/retroviral/lentiviral (e.g. SIV) vectors and/or non-viral nucleic acid molecules (e.g. plasmids, mRNAs or saRNAs) of the invention may be administered by any appropriate route. It may be desired to direct the compositions of the present invention (as described above) to the respiratory system of a subject. Efficient transmission of a therapeutic/prophylactic composition or medicament to the site of infection in the respiratory tract may be achieved by oral or intra-nasal administration, for example, as aerosols (e.g. nasal sprays), or by catheters. Typically the viral/retroviral/lentiviral (e.g. SIV) vectors and/or non-viral nucleic acid molecules (e.g. plasmids, mRNAs or saRNAs) of the invention are stable in clinically relevant nebulisers, inhalers (including metered dose inhalers), catheters and aerosols, etc. Typically, therefore, the viral/retroviral/lentiviral (e.g. SIV) vectors and/or non-viral nucleic acid molecules (e.g. plasmids, mRNAs or saRNAs) of the invention are formulated for administration to the lungs by any appropriate means, e.g. they may be formulated for intratracheal administration (e.g. intratracheal instillation), intranasal administration (e.g. intranasal instillation), aerosol delivery, nebulization, or direct injection or delivery to the lungs (e.g. delivered by catheter). Other modes of delivery, e.g. intravenous delivery, are also encompassed by the invention.

In some embodiments the nose is a preferred production site for a therapeutic protein using a viral/retroviral/lentiviral (e.g. SIV) vector and/or non-viral nucleic acid molecule (e.g. plasmid, mRNA or saRNA) of the invention for at least one of the following reasons: (i) extracellular barriers such as inflammatory cells and sputum are less pronounced in the nose; (ii) ease of vector administration; (iii) smaller quantities of vector/nucleic acid required; and (iv) ethical considerations. Thus, transduction of nasal epithelial cells with a viral/retroviral/lentiviral (e.g. SIV) vector or transfection with a non-viral nucleic acid molecule (e.g. plasmid, mRNA or saRNA) of the invention may result in efficient expression of the therapeutic GM-CSF transgene, as described herein. Accordingly, nasal administration of a viral/retroviral/lentiviral (e.g. SIV) vector and/or non-viral nucleic acid molecule (e.g. plasmid, mRNA or saRNA) of the invention may be preferred.

Formulations for intra-nasal administration may be in the form of nasal droplets or a nasal spray. An intra-nasal formulation may comprise droplets having approximate diameters in the range of 1-5000 μm, such as 500-4000 μm, 1000-3000 μm, 100-1000 μm, less than 500 μm, less than 400 μm, less than 300 μm, less than 250 μm, less than 200 μm, less than 100 μm, less than 75 μm, less than 50 μm, less than 25 μm, less than 20 μm, less than 15 μm, less than 12.5 μm, less than 10 μm, less than 5 μm, less than 2.5 μm or smaller. Alternatively, in terms of volume, the droplets may be in the range of about 0.001-100 μl, such as 0.1-50 μl or 1.0-25 μl, or such as 0.001-1 μl.

An aerosolised pharmaceutical composition of the invention may be characterised as having a droplet size having a Mass Median Aerodynamic Diameter (MMAD) of less than 5 μm, and having a Fine Particle Fraction (FPF defined as the proportion of aerosol contained within droplets with MMADs less than 5 μm) greater than 50%; and having greater than 50% of the total aerosolised plasmid delivered intact.

Mass Median Aerodynamic Diameter (MMAD) is a well known means of characterizing droplet size in an aerosol. The measurement, with the geometric standard deviation, used to describe the droplet size distribution of any aerosol statistically, based on the weight and size of the droplets. Means of calculating the MMAD of an aerosol are well known in the art.

Fine Particle Fraction is a measure of the proportion of droplets having the desired size characteristic. For the present invention, this is defined as the proportion of aerosol contained in droplets of between 1-3 μm in diameter. Again, means of calculating the Fine Particle Fraction of an aerosol are well known in the art.

Preferably an aerosolised pharmaceutical composition is formulated as an aerosol, wherein the aerosol has a droplet size having a MMAD in the range 1-3 m, and having a FPF greater than 50%; and having greater than 50% of the total aerosolised plasmid delivered intact. More preferably the aerosol has a MMAD in the range 1-3 μm and a FPF greater than 60%.

The aerosol formulation may take the form of a powder, suspension or solution. The size of aerosol droplets is relevant to the delivery capability of an aerosol. Smaller droplets may travel further down the respiratory airway towards the alveoli than would larger droplets. In one embodiment, the aerosol droplets have a diameter distribution to facilitate delivery along the entire length of the bronchi, bronchioles, and alveoli. Alternatively, the droplet size distribution may be selected to target a droplets section of the respiratory airway, for example the alveoli. In the case of aerosol delivery of the medicament, the droplets may have diameters in the approximate range of 0.1-50 μm, preferably 1-25 μm, more preferably 1-3 μm.

Aerosol droplets may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J. in Remington's Pharmaceutical Sciences (supra). The agents may be formulated as solution aerosols, dispersion or suspension aerosols of dry powders, emulsions or semisolid preparations. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising a vector of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

In some cases after an initial administration a subsequent administration of a viral/retroviral/lentiviral (e.g. SIV) vector and/or non-viral nucleic acid molecule (e.g. plasmid, mRNA or saRNA) may be performed. The administration may, for instance, be at least six months, eight months, ten months, a year or more after the initial administration. In some instances, a viral/retroviral/lentiviral (e.g. SIV) vector and/or non-viral nucleic acid molecule (e.g. plasmid, mRNA or saRNA) of the invention may be administered at least every six months, annually or at longer intervals. Preferably, administration is every six months, more preferably annually. The viral/retroviral/lentiviral (e.g. SIV) vector and/or non-viral nucleic acid molecule (e.g. plasmid, mRNA or saRNA) may, for instance, be administered at intervals dictated by when the effects of the previous administration are decreasing, and when an additional administration will not exceed the therapeutic window and/or be associated with one or more histopathological change as described herein.

Any two or more viral/retroviral/lentiviral (e.g. SIV) vectors and/or non-viral nucleic acid molecules (e.g. plasmids, mRNAs or saRNAs) of the invention may be administered separately, sequentially or simultaneously. Thus two or more viral/retroviral/lentiviral (e.g. SIV) vectors and/or non-viral nucleic acid molecules (e.g. plasmids, mRNAs or saRNAs), wherein at least one viral/retroviral/lentiviral (e.g. SIV) vector and/or non-viral nucleic acid molecule (e.g. plasmid, mRNA or saRNA) is a viral/retroviral/lentiviral (e.g. SIV) vector and/or non-viral nucleic acid molecule (e.g. plasmid, mRNA or saRNA) of the invention, may be administered separately, simultaneously or sequentially. In particular two or more viral/retroviral/lentiviral (e.g. SIV) vectors and/or non-viral nucleic acid molecules (e.g. plasmids, mRNAs or saRNAs) of the invention may be administered in such a manner. The two may be administered in the same or different compositions. In a preferred instance, the two viral/retroviral/lentiviral (e.g. SIV) vectors and/or non-viral nucleic acid molecules (e.g. plasmids, mRNAs or saRNAs) may be delivered in the same composition.

Treatment according to the invention preferably comprises administering the medicament as an aerosol to a patient in need thereof. Breath-actuated nebulisers may preferably used when administering an aerosolised medicament to the patient. This is because the breath enhancement mechanism increases the proportion of aerosol generated during patient inhalation. Preferably the aerosol may be generated from a breath-actuated nebuliser device with a formulation capacity of between 2 ml and 10 ml. Preferably the breath-actuated nebulizer is capable of generating stable formulation aerosols for the duration of aerosol delivery. By “stable aerosol generation” we include where the aerosols have the physical characteristics described above. Also, it may be preferred that the aerosol is delivered to a patient at an aerosol delivery rate of between 80 μl/min and 400 μl/min, assessed under standard simulated breathing conditions (sinusoidal breathing, tidal volume 500 ml and inspiratory:expiratory ratio of 1:1).

### Animal Models

Existing animal models for PAP all have disadvantages that limit their utility as models for PAP, particularly aPAP. The most common current animal model for PAP is a GM-CSF knock out (KO) mice. However, GM-CSF KO mice do not express anti-GM-CSF autoantibodies and it is currently unclear whether efficacy and/or toxicity may be affected by the presence of such autoantibodies. Some previous studies have shown that treatment of non-human primates to anti-GM-CSF antibodies can induce aPAP-like phenotype. However this model is too complex for early proof of concept studies. Rasgrp1-deficient mice develop autoantibody-mediated PAP. However, the phenotype only develops in older mice (^{˜}12 months) and is associated with high mortality which makes this model difficult to work with.

Accordingly, the present invention provides a new animal model for PAP, specifically aPAP which overcomes one or more of the problems associated with conventional PAP animal models. In particular, the present inventions have developed a murine model by passive immunisation of mice with anti-mGM-CSF antibodies.

In particular, the invention provides a rodent model for aPAP, wherein said rodent has been passively immunised with anti-GM-CSF antibodies by intranasal administration.

Typically the rodent is a mouse. The genetic background of the mouse is not limited. Non-limiting examples of mouse strains which may be used in a mouse model of the invention include, C57 black 6 background mice, wild-type mice or any strain of mice with a relevant genetic modification, such as a GM-CSF knock out mouse.

The anti-GM-CSF antibodies used in a model of the invention are not limited. When the model is a mouse model, murine anti-GM-CSF antibodies may typically be used. Non-limiting examples of murine anti-GM-CSF antibodies which may be used include MMGM-CSF A7.39 and MMGM-CSF B2.6, which are described in Uyttenhove et al. (Eur. J. Immunol. 2018. 48:1883-1891), which is herein incorporated by reference in its entirety. Other non-limiting examples of anti-GM-CSF antibodies include GCA21, GCA7 and GCB59, which are described in Piccoli et al. (Nat. Comms. 2015. 6:7375), which is herein incorporated by reference in its entirety.

Typically a rodent model of aPAP achieves a BALF concentration of anti-GM-CSF antibodies of at least about 2 μg/mL, such as at least about 3 μg/mL, at least about 4 μg/mL, at least about 5 μg/mL, at least about 6 μg/mL, at least about 7 μg/mL, at least about 8 μg/mL, at least about 9 μg/mL, at least about 10 μg/mL or greater, preferably at least about 4 μg/mL. Non-limiting examples of BALF concentration of anti-GM-CSF antibodies that may be achieved in a rodent model of the invention include between about 1-10 μg/mL, such as between about 2-7 μg/mL, between about 4-6 μg/mL or greater, preferably between about 4-6 μg/mL.

The invention also provides a method of generating a rodent model for aPAP, comprising administration of anti-GM-CSF antibodies to a rodent by intranasal administration. Any and all disclosure herein in relation to a rodent model of the invention applies equally and without reservation to method of generating a rodent model of the invention.

A rodent model of the invention, particularly a mouse model, as described herein may be useful for studying aPAP. A rodent model of the invention, particularly a mouse model, as described herein may be useful for studying pharmaceuticals, cell products, biologics or small molecules intended for the treatment of aPAP, optionally studying compositions of the invention.

Preferably the anti-GM-CSF antibodies used in the rodent model of the invention are prepared in a pure form and/or at a high concentration prior to passive immunisation. By way of non-limiting example, one or more anti-GM-CSF antibody used in the rodent model of the invention may be prepared at a concentration of at least 600 μg/mL, such as at least 700 μg/mL, at least 750 μg/mL, at least 800 μg/mL, or more, such as at about 820 μg/mL. Alternatively or in addition, one or more anti-GM-CSF antibody used for passive immunisation in the rodent model of the invention may have less than 0.1 ng endotoxin per mg antibody, such as less than 0.09 ng endotoxin per mg antibody, less than 0.08 ng endotoxin per mg antibody, less than 0.08 ng endotoxin per mg antibody, or less than 0.07 ng endotoxin per mg antibody, such as less 0.06 ng endotoxin per mg antibody.

Anti-GM-CSF antibodies may be detectable in the BALF of the rodent model for between about 1 to about 30 days, such as between about 1 to about 20 days, between about 5 to about 20 days, between about 1 to about 15 days, between about 5 to about 10 days or between about 1 to about 10 days, following passive immunisation. Alternatively or additionally, the concentration of anti-GM-CSF antibodies in the BALF of the rodent model may be at least about 5 ng/mL, such as at least about 6 ng/mL, at least about 7 ng/mL, at least about 8 ng/mL, at least about 9 ng/mL, at least about 10 ng/mL, at least about 11 ng/mL, at least about 12 ng/mL, at least about 13 ng/mL, at least about 14 ng/mL, at least about 15 ng/mL, or more.

A rodent model of PAP, specifically aPAP, may be generated by the administration of one or more anti-GM-CSF antibody at a dose of between about 1 μg/mouse to about 100 μg/mouse, such as between about 1 μg/mouse to about 50 μg/mouse, between about 5 μg/mouse to about 50 μg/mouse, between about 10 μg/mouse to about 40 μg/mouse. Where multiple anti-GM-CSF antibodies are administered to generate the model, the dose of each antibody to be administered may be determined independently. By way of non-limiting example, a rodent model of PAP, specifically aPAP, may be generated by the administration of MMGM-CSF A7.39 and MMGM-CSF B2.6, wherein optionally MMGM-CSF A7.39 is administered at a dose of 40 μg/mouse and MMGM-CSF B2.639 is administered at a dose of 10 μg/mouse.

As demonstrated herein, in some instances repeated passive immunisations may be required to maintain the anti-GM-CSF antibodies within a desired concentration range in an individual model subject. By way of non-limiting example, said one or more anti-GM-CSF antibody may be administered daily, every 2 days, every 3 days, every 4 days, every 5 days, every 6 days, weekly, every 10 days, every 2 weeks, or monthly. In some preferred embodiments, said one or more anti-GM-CSF antibody may be administered weekly. By way of example, a rodent model of PAP, specifically aPAP, may be generated by weekly administration of MMGM-CSF A7.39 and MMGM-CSF B2.6, wherein optionally MMGM-CSF A7.39 is administered at a dose of 40 μg/mouse at each administration and MMGM-CSF B2.639 is administered at a dose of 10 μg/mouse at each administration.

Repeated administration of the antibody may be carried out for the duration of an experiment being conducted on the model subject requires. For example, if an experiment is being carried out over a period of 10 months, then said one or more anti-GM-CSF antibody may be administered (e.g. weekly) for the 10-month duration of the experiment.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

ALIGNMENT SCORES FOR DETERMINING SEQUENCE IDENTITY
A R N D C Q E G H I L K M F P S T W Y V

A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

The percent identity is then calculated as:

$$\frac{Total number of identical matches}{[ length of the longer sequence plus the number of gaps introduced into the longer sequence in order to align the two sequences ]} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4-hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labelling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### Sequence Information

SEQ ID NO: 1 hGM-CSF amino acid sequence (UniProt Accession No. P04141)
SEQ ID NO: 2 hGM-CSF nucleic acid sequence (Genbank Accession No. M11220.1)
SEQ ID NO: 3 mGM-CSF amino acid sequence (UniProt Accession No. P01587)
SEQ ID NO: 4 mGM-CSF nucleic acid sequence (GenBank Accession No. AY950559.1)
SEQ ID NO: 5 mGM-CSF transgene sequence comprised in pIC017 and pIC098
SEQ ID NO: 6 exemplary hCEF promoter
SEQ ID NO: 7 exemplary CMV promoter
SEQ ID NO: 8 exemplary EF1a promoter
SEQ ID NO: 9 β-globin/IgG chimeric intron comprising a SIV RRE
SEQ ID NO: 10 pIC017 hCEF mGMCSF plasmid
SEQ ID NO: 11 pIC098 CMV mGMCSF plasmid
SEQ ID NO: 12 exemplary WPRE sequence
SEQ ID NO: 13 exemplary mifepristone-regulated promoter
SEQ ID NO: 14 exemplary trans-activator for use with a mifepristone-regulated promoter
SEQ ID NO: 15 pDNA1 plasmid pSIV-2V-GMCSF (FIG. 2B)
SEQ ID NO: 16 pDNA2a plasmid pGM691 (FIG. 2D)
SEQ ID NO: 17 pDNA2a plasmid pGM297 (FIG. 2E)
SEQ ID NO: 18 pDNA2b plasmid pGM299 (FIG. 2F)
SEQ ID NO: 19 pDNA3a plasmid pGM301 (FIG. 2G)
SEQ ID NO: 20 pDNA3b plasmid pGM303 (FIG. 2H)
SEQ ID NO: 21 pDNA1* plasmid pSIV-2V-Transactivator (FIG. 2C)
SEQ ID NO: 22 pDNA1^{ta+} plasmid pSIV-1V-GMCSF (FIG. 2A)
SEQ ID NO: 23 pDNA3 plasmid pMD2.G (FIG. 2I)
SEQ ID NO: 24 codon-optimised gag-pol genes (from pGM691)
SEQ ID NO: 25 exemplary CAG promoter
SEQ ID NO: 26 preferred exemplary CMV promoter

SEQ ID NO: 1 hGM-CSF amino acid sequence (UniProt Accession No. P04141)
MWLQSLLLLGTVACSISAPARSPSPSTQPWEHVNAIQEARRLLNLSRDTAAEMNETVEVISEMFDLQEPTCLQTR
LELYKQGLRGSLTKLKGPLTMMASHYKQHCPPTPETSCATQIITFESFKENLKDFLLVIPFDCWEPVQE
SEQ ID NO: 2 hGM-CSF nucleic acid sequence (Genbank Accession No. M11220.1)
acacagagag aaaggctaaa gttctctgga ggatgtggct gcagagcctg ctgctcttgg gcactgtggc
ctgcagcatc tctgcacccg cccgctcgcc cagccccagc acgcagccct gggagcatgt gaatgccatc
caggaggccc ggcgtctcct gaacctgagt agagacactg ctgctgagat gaatgaaaca gtagaagtca
tctcagaaat gtttgacctc caggagccga cctgcctaca gacccgcctg gagctgtaca agcagggcct
gcggggcagc ctcaccaagc tcaagggccc cttgaccatg atggccagcc actacaagca gcactgccct
ccaaccccgg aaacttcctg tgcaacccag attatcacct ttgaaagttt caaagagaac ctgaaggact
ttctgcttgt catccccttt gactgctggg agccagtcca ggagtgagac cggccagatg aggctggcca
agccggggag ctgctctctc atgaaacaag agctagaaac tcaggatggt catcttggag ggaccaaggg
gtgggccaca gccatggtgg gagtggcctg gacctgccct gggcacactg accctgatac aggcatggca
gaagaatggg aatattttat actgacagaa atcagtaata tttatatatt tatattttta aaatatttat
ttatttattt atttaagttc atattccata tttattcaag atgttttacc gtaataatta ttattaaaaa
tagcttctaa aaaaaaaaa
SEQ ID NO: 3 mGM-CSF amino acid sequence (UniProt Accession No. P01587)
MWLQNLLFLGIVVYSLSAPTRSPITVTRPWKHVEAIKEALNLLDDMPVTLNEEVEVVSNEFSFKKLTCVQTRLKI
FEQGLRGNFTKLKGALNMTASYYQTYCPPTPETDCETQVTTYADFIDSLKTFLTDIPFECKKPGQK
SEQ ID NO: 4 mGM-CSF amino acid sequence (Genbank Accession No. AY950559.1)
atggctcacg agcggaaggc taaggtgctg cgcagaatgt ggctgcagaa cctgctgttc ctgggcatcg
tggtgtacag cctgagcgcc cccaccagaa gccccatcac cgtgaccaga ccctggaagc acgtggaggc
catcaaggaa gctctgaacc tgctggacga catgcccgtg accctgaacg aggaggtgga ggtggtgagc
aacgagttta gctttaagaa gctgacctgc gtgcagaccc ggctgaagat cttcgagcag ggactgcggg
gcaactttac caagctgaag ggagccctga acatgaccgc cagctactac cagacctact gccctcccac
acccgagacc gactgtgaaa cccaggtgac cacctacgcc gactttatcg acagcctgaa gaccttcctg
accgacatcc ccttcgagtg taagaagccc gtgcagaagt gactcgagcg g
SEQ ID NO: 5 mGM-CSF transgene sequence comprised in pIC017 and pIC098
ctagccacca tgtggctgca gaacctgctg ttcctgggca ttgtggtgta cagcctgtct
gcccctacaa gatcccctat cacagtgacc agaccttgga aacatgtgga agccatcaaa
gaggccctga atctgctgga tgacatgcct gtgacactga atgaagaggt ggaagtggtg
tccaatgagt tcagcttcaa gaaactgacc tgtgtgcaga ccaggctgaa gatttttgag
cagggcctga gaggcaactt caccaagctg aaaggggctc tgaacatgac agccagctac
taccagacct actgtcctcc tacacctgag acagactgtg aaacccaagt gaccacctat
gctgacttca ttgacagcct caagaccttc ctgacagaca tcccctttga gtgcaagaaa
cctggccaga agtgagggcc
SEQ ID NO: 6 exemplary hCEF promoter

| | |
|---|---|
| agatctgtta cataacttat ggtaaatggc ctgcctggct gactgcccaa tgacccctgc | 60 |
| ccaatgatgt caataatgat gtatgttccc atgtaatgcc aatagggact ttccattgat | 120 |
| gtcaatgggt ggagtattta tggtaactgc ccacttggca gtacatcaag tgtatcatat | 180 |
| gccaagtatg ccccctattg atgtcaatga tggtaaatgg cctgcctggc attatgccca | 240 |
| gtacatgacc ttatgggact ttcctacttg gcagtacatc tatgtattag tcattgctat | 300 |
| taccatggga attcactagt ggagaagagc atgcttgagg gctgagtgcc cctcagtggg | 360 |
| cagagagcac atggcccaca gtccctgaga agttgggggg aggggtgggc aattgaactg | 420 |
| gtgcctagag aaggtggggc ttgggtaaac tgggaaagtg atgtggtgta ctggctccac | 480 |
| ctttttcccc agggtggggg agaaccatat ataagtgcag tagtctctgt gaacattcaa | 540 |
| gcttctgcct tctccctcct gtgagtttgc tagc | 574 |

SEQ ID NO: 7 exemplary CMV promoter

| | |
|---|---|
| ccgcggagat ctcaatattg gccattagcc atattattca ttggttatat agcataaatc | 60 |
| aatattggct attggccatt gcatacgttg tatctatatc ataatatgta catttatatt | 120 |
| ggctcatgtc caatatgacc gccatgttgg cattgattat tgactagtta ttaatagtaa | 180 |
| tcaattacgg ggtcattagt tcatagccca tatatggagt tccgcgttac ataacttacg | 240 |
| gtaaatggcc cgcctggctg accgcccaac gacccccgcc cattgacgtc aataatgacg | 300 |
| tatgttccca tagtaacgcc aatagggact ttccattgac gtcaatgggt ggagtattta | 360 |
| cggtaaactg cccacttggc agtacatcaa gtgtatcata tgccaagtcc gccccctatt | 420 |
| gacgtcaatg acggtaaatg gcccgcctgg cattatgccc agtacatgac cttacgggac | 480 |
| tttcctactt ggcagtacat ctacgtatta gtcatcgcta ttaccatggt gatgcggttt | 540 |
| tggcagtaca ccaatgggcg tggatagcgg tttgactcac ggggatttcc aagtctccac | 600 |
| cccattgacg tcaatgggag tttgttttgg caccaaaatc aacgggactt tccaaaatgt | 660 |
| cgtaataacc ccgccccgtt gacgcaaatg ggcggtaggc gtgtacggtg ggaggtctat | 720 |
| ataagcagag ctcgtttagt gaaccgtcag atcactagaa gctttattgc ggtagtttat | 780 |
| cacagttaaa ttgctaacgc agtcagtgct tctgacacaa cagtctcgaa cttaagctgc | 840 |
| agaagttggt cgtgaggcac tgggcaggct agc | 873 |

SEQ ID NO: 8 exemplary EF1a promoter

| | |
|---|---|
| agatccatat ccgcggcaat tttaaaagaa agggaggaat agggggacag acttcagcag | 60 |
| agagactaat taatataata acaacacaat tagaaataca acatttacaa accaaaattc | 120 |
| aaaaaatttt aaattttaga gccgcggaga tcccgtgagg ctccggtgcc cgtcagtggg | 180 |
| cagagcgcac atcgcccaca gtccccgaga agttgggggg aggggtcggc aattgaaccg | 240 |
| gtgcctagag aaggtggcgc ggggtaaact gggaaagtga tgtcgtgtac tggctccgcc | 300 |
| tttttcccga gggtggggga gaaccgtata taagtgcagt agtcgccgtg aacgttcttt | 360 |
| ttcgcaacgg gtttgccgcc agaacacagg ctagc | 395 |

SEQ ID NO: 9 β-globin/IgG chimeric intron comprising a SIV RRE
GTAAGTATCAAGGTTACAAGACAGGTTTAAGGAGACCAATAGAAACTGGGCTTGTCGAGACAGAGAAG
ACTCTTGCGTTTCTGATAGGCACGCGGCCGCCCGTTTGTGCTAGGGTTCTTAGGCTTCTTGGGGGCTG
CTGGAACTGCAATGGGAGCAGCGGCGACAGCCCTGACGGTCCAGTCTCAGCATTTGCTTGCTGGGATA
CTGCAGCAGCAGAAGAATCTGCTGGCGGCTGTGGAGGCTCAACAGCAGATGTTGAAGCTGACCATTTG
GGGTGTTAAAAACCTCAATGCCCGCGTCACAGCCCTTGAGAAGTACCTAGAGGATCAGGCACGACTAA
ACTCCTGGGGGTGCGCATGGAAACAAGTATGTCATACCACAGTGGAGTGGCCCTGGACAAATCGGACT
CCGGATTGGCAAAATATGACTTGGTTGGAGTGGGAAAGACAAATAGCTGATTTGGAAAGCAACATTAC
GAGACAATTAGTGAAGGCTAGAGAACAAGAGGAAAAGAATCTAGATGCCTATCAGAAGTTAACTAGTT
GGTCAGATTTCTGGTCTTGGTTCGATTTCTCAAAATGGCTTAACATTTTAAAAATGGGATTTTTAGTA
ATAGTAGGAATAATAGGGTTAAGATTACTTTACACAGTATATGGATGTATAGTGAGGGTTAGGCAGGG
ATATGTTCCTCTATCTCCACAGATCCATATGCGGCCGCCTATTGGTCTTACTGACATCCACTTTGCCT
TTCTCTCCACAG
Underlined = β-globin/IgG chimeric intron
Double underlined = NotI restriction sites
Italicised = SIV RRE sequence
SEQ ID NO: 10 pIC017 hCEF GMCSF plasmid
agatctgtta cataacttat ggtaaatggc ctgcctggct gactgcccaa tgacccctgc
ccaatgatgt caataatgat gtatgttccc atgtaatgcc aatagggact ttccattgat
gtcaatgggt ggagtattta tggtaactgc ccacttggca gtacatcaag tgtatcatat
gccaagtatg ccccctattg atgtcaatga tggtaaatgg cctgcctggc attatgccca
gtacatgacc ttatgggact ttcctacttg gcagtacatc tatgtattag tcattgctat
taccatggga attcactagt ggagaagagc atgcttgagg gctgagtgcc cctcagtggg
cagagagcac atggcccaca gtccctgaga agttgggggg aggggtgggc aattgaactg
gtgcctagag aaggtggggc ttgggtaaac tgggaaagtg atgtggtgta ctggctccac
ctttttcccc agggtggggg agaaccatat ataagtgcag tagtctctgt gaacattcaa
gcttctgcct tctccctcct gtgagtttgg taagtcactg actgtctatg cctgggaaag
ggtgggcagg agatggggca gtgcaggaaa agtggcacta tgaaccctgc agccctagga
atgcatctag acaattgtac taaccttctt ctctttcctc tcctgacagg ttggtgtaca
gtagcttgct agccaccatg tggctgcaga acctgctgtt cctgggcatt gtggtgtaca
gcctgtctgc ccctacaaga tcccctatca cagtgaccag accttggaaa catgtggaag
ccatcaaaga ggccctgaat ctgctggatg acatgcctgt gacactgaat gaagaggtgg
aagtggtgtc caatgagttc agcttcaaga aactgacctg tgtgcagacc aggctgaaga
tttttgagca gggcctgaga ggcaacttca ccaagctgaa aggggctctg aacatgacag
ccagctacta ccagacctac tgtcctccta cacctgagac agactgtgaa acccaagtga
ccacctatgc tgacttcatt gacagcctca agaccttcct gacagacatc ccctttgagt
gcaagaaacc tggccagaag tgagggccct gtgccttcta gttgccagcc atctgttgtt
tgcccctccc ctgtgccttc cttgaccctg gaaggtgcca ctcccactgt cctttcctaa
taaaatgagg aaattgcatt gcattgtctg agtaggtgtc attctattct ggggggtggg
gtggggcagg acagcaaggg ggaggattgg gaagacaata gcaggcatgc agatcagcag
ttcaacctgt tgatagtatg tactaagctc tcatgtttaa tgtactaagc tctcatgttt
aatgaactaa accctcatgg ctaatgtact aagctctcat ggctaatgta ctaagctctc
atgtttcatg tactaagctc tcatgtttga acaataaaat taatataaat cagcaactta
aatagcctct aaggttttaa gttttataag aaaaaaaaga atatataagg cttttaaagg
ttttaaggtt tcctaggtta tcctggtacc ttagaaaaac tcatccagca tcaaatgaaa
ctgcaattta ttcatatcag gattatcaat accatatttt tgaaaaagtc ttttctgtaa
tgaaggagaa aactcaccca ggcagttcca taggatggca agatcctggt atctgtctgc
aattccaact cttccaacat caatacaacc tattaatttc ccctcatcaa aaataaggtt
atcaagtgag aaatcaccat gagtgaccac tgaatctggt gagaatggca aaagcttatg
catttctttc cagacttgtt caacaggcca gccatttctc tcatcatcaa aatcactggc
atcaaccaaa ccattattca ttcttgattg ggcctgagcc agtctaaata ctctatcaga
gttaaaagga caattacaaa caggaatgga atgcaatctt ctcaggaaca ctgccagggc
atcaacaata ttttcacctg aatcaggata ttcttctaat acctggaatg ctgttttccc
tgggatggca gtggtgagta accatgcatc atcaggagtt ctgataaaat gcttgatggt
tggaagaggc ataaattcag tcagccagtt tagtctgacc atctcatctg taacatcatt
ggcaacagaa cctttgccat gtttcagaaa caactctggg gcatctggct tcccatacaa
tctatagatt gtggcacctg attgcccaac attatctcta gcccatttat acccatataa
atcagcatcc atgttggaat ttaatcttgg cctggagcaa gaggtttctc tttgaatatg
gctcatggat cccctcctat agtgagttgt attatactat gcagatatac tatgccaatg
tttaattgtc aa
SEQ ID NO: 11 pIC098 CMV GMCSF plasmid
GGCATTGATT ATTGACTAGT TATTAATAGT AATCAATTAC GGGGTCATTA GTTCATAGCC
CATATATGGA GTTCCGCGTT ACATAACTTA CGGTAAATGG CCCGCCTGGC TGACCGCCCA
ACGACCCCCG CCCATTGACG TCAATAATGA CGTATGTTCC CATAGTAACG CCAATAGGGA
CTTTCCATTG ACGTCAATGG GTGGAGTATT TACGGTAAAC TGCCCACTTG GCAGTACATC
AAGTGTATCA TATGCCAAGT CCGCCCCCTA TTGACGTCAA TGACGGTAAA TGGCCCGCCT
GGCATTATGC CCAGTACATG ACCTTACGGG ACTTTCCTAC TTGGCAGTAC ATCTACGTAT
TAGTCATCGC TATTACCATG GTGATGCGGT TTTGGCAGTA CACCAATGGG CGTGGATAGC
GGTTTGACTC ACGGGGATTT CCAAGTCTCC ACCCCATTGA CGTCAATGGG AGTTTGTTTT
GGCACCAAAA TCAACGGGAC TTTCCAAAAT GTCGTAATAA CCCCGCCCCG TTGACGCAAA
TGGGCGGTAG GCGTGTACGG TGGGAGGTCT ATATAAGCAG AGCTCGTTTA GTGAACCGTC
AGATCACTAG AAGCTTTATT GCGGTAGTTT ATCACAGTTA AATTGCTAAC GCAGTCAGTG
CTTCTGACAC AACAGTCTCG AACTTAAGCT GCAGAAGTTG GTCGTGAGGC ACTGGGCAGG
TAAGTATCAA GGTTACAAGA CAGGTTTAAG GAGACCAATA GAAACTGGGC TTGTCGAGAC
AGAGAAGACT CTTGCGTTTC TGATAGGCAC CTATTGGTCT TACTGACATC CACTTTGCCT
TTCTCTCCAC AGGTGTCCAC TCCCAGTTCA ATTACAGCTC TTAAGGCTAG AGTACTTAAT
ACGACTCACT ATAGGCTAGc tagccaccat gtggctgcag aacctgctgt tcctgggcat
tgtggtgtac agcctgtctg cccctacaag atcccctatc acagtgacca gaccttggaa
acatgtggaa gccatcaaag aggccctgaa tctgctggat gacatgcctg tgacactgaa
tgaagaggtg gaagtggtgt ccaatgagtt cagcttcaag aaactgacct gtgtgcagac
caggctgaag atttttgagc agggcctgag aggcaacttc accaagctga aaggggctct
gaacatgaca gccagctact accagaccta ctgtcctcct acacctgaga cagactgtga
aacccaagtg accacctatg ctgacttcat tgacagcctc aagaccttcc tgacagacat
cccctttgag tgcaagaaac ctggccagaa gtgagggccc tgtgccttct agttgccagc
catctgttgt ttgcccctcc cctgtgcctt ccttgaccct ggaaggtgcc actcccactg
tcctttccta ataaaatgag gaaattgcat tgcattgtct gagtaggtgt cattctattc
tggggggtgg ggtggggcag gacagcaagg gggaggattg ggaagacaat agcaggcatg
cagatcagca gttcaacctg ttgatagtat gtactaagct ctcatgttta atgtactaag
ctctcatgtt taatgaacta aaccctcatg gctaatgtac taagctctca tggctaatgt
actaagctct catgtttcat gtactaagct ctcatgtttg aacaataaaa ttaatataaa
tcagcaactt aaatagcctc taaggtttta agttttataa gaaaaaaaag aatatataag
gcttttaaag gttttaaggt ttcctaggtt atcctggtac cttagaaaaa ctcatccagc
atcaaatgaa actgcaattt attcatatca ggattatcaa taccatattt ttgaaaaagt
cttttctgta atgaaggaga aaactcaccc aggcagttcc ataggatggc aagatcctgg
tatctgtctg caattccaac tcttccaaca tcaatacaac ctattaattt cccctcatca
aaaataaggt tatcaagtga gaaatcacca tgagtgacca ctgaatctgg tgagaatggc
aaaagcttat gcatttcttt ccagacttgt tcaacaggcc agccatttct ctcatcatca
aaatcactgg catcaaccaa accattattc attcttgatt gggcctgagc cagtctaaat
actctatcag agttaaaagg acaattacaa acaggaatgg aatgcaatct tctcaggaac
actgccaggg catcaacaat attttcacct gaatcaggat attcttctaa tacctggaat
gctgttttcc ctgggatggc agtggtgagt aaccatgcat catcaggagt tctgataaaa
tgcttgatgg ttggaagagg cataaattca gtcagccagt ttagtctgac catctcatct
gtaacatcat tggcaacaga acctttgcca tgtttcagaa acaactctgg ggcatctggc
ttcccataca atctatagat tgtggcacct gattgcccaa cattatctct agcccattta
tacccatata aatcagcatc catgttggaa tttaatcttg gcctggagca agaggtttct
ctttgaatat ggctcatgga tcccctccta tagtgagttg tattatacta tgcagatata
ctatgccaat gtttaattgt caa
SEQ ID NO: 12 exemplary WPRE sequence

| | |
|---|---|
| gggcccaatc aacctctgga ttacaaaatt tgtgaaagat tgactggtat tcttaactat | 60 |
| gttgctcctt ttacgctatg tggatacgct gctttaatgc ctttgtatca tgctattgct | 120 |
| tcccgtatgg ctttcatttt ctcctccttg tataaatcct ggttgctgtc tctttatgag | 180 |
| gagttgtggc ccgttgtcag gcaacgtggc gtggtgtgca ctgtgtttgc tgacgcaacc | 240 |
| cccactggtt ggggcattgc caccacctgt cagctccttt ccgggacttt cgctttcccc | 300 |
| ctccctattg ccacggcgga actcatcgcc gcctgccttg cccgctgctg gacaggggct | 360 |
| cggctgttgg gcactgacaa ttccgtggtg ttgtcgggga aatcatcgtc ctttccttgg | 420 |
| ctgctcgcct gtgttgccac ctggattctg cgcgggacgt ccttctgcta cgtcccttcg | 480 |
| gccctcaatc cagcggacct tccttcccgc ggcctgctgc cggctctgcg gcctcttccg | 540 |
| cgtcttcgcc ttcgccctca gacgagtcgg atctcccttt gggccgcctc cccgcaagct | 600 |

SEQ ID NO: 13 exemplary mifepristone-regulated promoter
ACCGAGCTCTTACGCGGGTCGAAGCGGAGTACTGTCCTCCGAGTGGAGTACTGTCCTCCGAGCGGAGTACTGTCC
TCCGAGTCGAGGGTCGAAGCGGAGTACTGTCCTCCGAGTGGAGTACTGTCCTCCGAGCGGAGTACTGTCCTCCGA
GTCGACTCTAGAGGGTATATAATGGATCTCGAGATATCGGAGCTCGTTTAGTGAACCGTCAGATCGCCTGGAGAC
GCCATCCACGCTGTTTTGACCTCCATAGAAGACACCGGGACCGATCCAGCCTCCGCGGCCGGGAACGGTGCATTG
GAACGCGCATTCCCCGTGTTAATTAACAGGTAAGTGTCTTCCTCCTGTTTCCTTCCCCTGCTATTCTGCTCAACC
TTCCTATCAGAAACTGCAGTATCTGTATTTTTGCTAGCAGTAATACTAACGGTTCTTTTTTTCTCTTCACAGGCC
ACCAAGCTTGGTACCGAGCTCGGATCCACTAGTCCAGTGTGGTGGAATTCTGCAGATCGAAACGATGATAGATCC
C
SEQ ID NO: 14 exemplary trans-activator for use with a mifepristone-regulated
promoter
ATGGACTCCCAGCAGCCAGATCTGAAGCTACTGTCTTCTATCGAACAAGCATGCGATATTTGCCGACTTAAAAAG
CTCAAGTGCTCCAAAGAAAAACCGAAGTGCGCCAAGTGTCTGAAGAACAACTGGGAGTGTCGCTACTCTCCCAAA
ACCAAAAGGTCTCCGCTGACTAGGGCACATCTGACAGAAGTGGAATCAAGGCTAGAAAGACTGGAACAGCTATTT
CTACTGATTTTTCCTCGAGAAGACCTTGACATGATTTTGAAAATGGATTCTTTACAGGATATAAAAGCATTGTTA
GAATTCCCGGGTGTCGACCAGAAAAAGTTCAATAAAGTCAGAGTTGTGAGAGCACTGGATGCTGTTGCTCTCCCA
CAGCCAGTGGGCGTTCCAAATGAAAGCCAAGCCCTAAGCCAGAGATTCACTTTTTCACCAGGTCAAGACATACAG
TTGATTCCACCACTGATCAACCTGTTAATGAGCATTGAACCAGATGTGATCTATGCAGGACATGACAACACAAAA
CCTGACACCTCCAGTTCTTTGCTGACAAGTCTTAATCAACTAGGCGAGAGGCAACTTCTTTCAGTAGTCAAGTGG
TCTAAATCATTGCCAGGTTTTCGAAACTTACATATTGATGACCAGATAACTCTCATTCAGTATTCTTGGATGAGC
TTAATGGTGTTTGGTCTAGGATGGAGATCCTACAAACACGTCAGTGGGCAGATGCTGTATTTTGCACCTGATCTA
ATACTAAATGAACAGCGGATGAAAGAATCATCATTCTATTCATTATGCCTTACCATGTGGCAGATCCCACAGGAG
TTTGTCAAGCTTCAAGTTAGCCAAGAAGAGTTCCTCTGTATGAAAGTATTGTTACTTCTTAATACAATTCCTTTG
GAAGGGCTACGAAGTCAAACCCAGTTTGAGGAGATGAGGTCAAGCTACATTAGAGAGCTCATCAAGGCAATTGGT
TTGAGGCAAAAAGGAGTTGTGTCGAGCTCACAGCGTTTCTATCAACTTACAAAACTTCTTGATAACTTGCATGAT
CTTGTCAAACAACTTCATCTGTACTGCTTGAATACATTTATCCAGTCCCGGGCACTGAGTGTTGAATTTCCAGAA
ATGATGTCTGAAGTTATTGCTGGGTCGACGCCCATGGAATTCCAGTACCTGCCAGATACAGACGATCGTCACCGG
ATTGAGGAGAAACGTAAAAGGACATATGAGACCTTCAAGAGCATCATGAAGAAGAGTCCTTTCAGCGGACCCACC
GACCCCCGGCCTCCACCTCGACGCATTGCTGTGCCTTCCCGCAGCTCAGCTTCTGTCCCCAAGCCAGCACCCCAG
CCCTATCCCTTTACGTCATCCCTGAGCACCATCAACTATGATGAGTTTCCCACCATGGTGTTTCCTTCTGGGCAG
ATCAGCCAGGCCTCGGCCTTGGCCCCGGCCCCTCCCCAAGTCCTGCCCCAGGCTCCAGCCCCTGCCCCTGCTCCA
GCCATGGTATCAGCTCTGGCCCAGGCCCCAGCCCCTGTCCCAGTCCTAGCCCCAGGCCCTCCTCAGGCTGTGGCC
CCACCTGCCCCCAAGCCCACCCAGGCTGGGGAAGGAACGCTGTCAGAGGCCCTGCTGCAGCTGCAGTTTGATGAT
GAAGACCTGGGGGCCTTGCTTGGCAACAGCACAGACCCAGCTGTGTTCACAGACCTGGCATCCGTCGACAACTCC
GAGTTTCAGCAGCTGCTGAACCAGGGCATACCTGTGGCCCCCCACACAACTGAGCCCATGCTGATGGAGTACCCT
GAGGCTATAACTCGCCTAGTGACAGGGGCCCAGAGGCCCCCCGACCCAGCTCCTGCTCCACTGGGGGCCCCGGGG
CTCCCCAATGGCCTCCTTTCAGGAGATGAAGACTTCTCCTCCATTGCGGACATGGACTTCTCAGCCCTGCTGAGT
CAGATCAGCTCCTAA
SEQ ID NO: 15 pDNA1 plasmid pSIV-2V-GMCSF (FIG. 2B)
GGTACCTCAATATTGGCCATTAGCCATATTATTCATTGGTTATATAGCATAAATCAATATTGGCTATTGGCCATT
GCATACGTTGTATCTATATCATAATATGTACATTTATATTGGCTCATGTCCAATATGACCGCCATGTTGGCATTG
ATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGCCCACTTGGC
AGTACATCAAGTGTATCATATGCCAAGTCCGCCCCCTATTGACGTCAATGACGGTAAATGGCCCGCCTGGCATTA
TGCCCAGTACATGACCTTACGGGACTTTCCTACTTGGCAGTACATCTACGTATTAGTCATCGCTATTACCATGGT
GATGCGGTTTTGGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTGCGATCGCCC
GCCCCGTTGACGCAAATGGGCGGTAGGCGTGTACGGTGGGAGGTCTATATAAGCAGAGCTCGCTGGCTTGTAACT
CAGTCTCTTACTAGGAGACCAGCTTGAGCCTGGGTGTTCGCTGGTTAGCCTAACCTGGTTGGCCACCAGGGGTAA
GGACTCCTTGGCTTAGAAAGCTAATAAACTTGCCTGCATTAGAGCTTATCTGAGTCAAGTGTCCTCATTGACGCC
TCACTCTCTTGAACGGGAATCTTCCTTACTGGGTTCTCTCTCTGACCCAGGCGAGAGAAACTCCAGCAGTGGCGC
CCGAACAGGGACTTGAGTGAGAGTGTAGGCACGTACAGCTGAGAAGGCGTCGGACGCGAAGGAAGCGCGGGGTGC
GACGCGACCAAGAAGGAGACTTGGTGAGTAGGCTTCTCGAGTGCCGGGAAAAAGCTCGAGCCTAGTTAGAGGACT
AGGAGAGGCCGTAGCCGTAACTACTCTTGGGCAAGTAGGGCAGGCGGTGGGTACGCAATGGGGGCGGCTACCTCA
GCACTAAATAGGAGACAATTAGACCAATTTGAGAAAATACGACTTCGCCCGAACGGAAAGAAAAAGTACCAAATT
AAACATTTAATATGGGCAGGCAAGGAGATGGAGCGCTTCGGCCTCCATGAGAGGTTGTTGGAGACAGAGGAGGGG
TGTAAAAGAATCATAGAAGTCCTCTACCCCCTAGAACCAACAGGATCGGAGGGCTTAAAAAGTCTGTTCAATCTT
GTGTGCGTGCTATATTGCTTGCACAAGGAACAGAAAGTGAAAGACACAGAGGAAGCAGTAGCAACAGTAAGACAA
CACTGCCATCTAGTGGAAAAAGAAAAAAGTGCAACAGAGACATCTAGTGGACAAAAGAAAAATGACAAGGGAATA
GCAGCGCCACCTGGTGGCAGTCAGAATTTTCCAGCGCAACAACAAGGAAATGCCTGGGTACATGTACCCTTGTCA
CCGCGCACCTTAAATGCGTGGGTAAAAGCAGTAGAGGAGAAAAAATTTGGAGCAGAAATAGTACCCATTTTTTTG
TTTCAAGCCCTATCGAATTCCCGTTTGTGCTAGGGTTCTTAGGCTTCTTGGGGGCTGCTGGAACTGCAATGGGAG
CAGCGGCGACAGCCCTGACGGTCCAGTCTCAGCATTTGCTTGCTGGGATACTGCAGCAGCAGAAGAATCTGCTGG
CGGCTGTGGAGGCTCAACAGCAGATGTTGAAGCTGACCATTTGGGGTGTTAAAAACCTCAATGCCCGCGTCACAG
CCCTTGAGAAGTACCTAGAGGATCAGGCACGACTAAACTCCTGGGGGTGCGCATGGAAACAAGTATGTCATACCA
CAGTGGAGTGGCCCTGGACAAATCGGACTCCGGATTGGCAAAATATGACTTGGTTGGAGTGGGAAAGACAAATAG
CTGATTTGGAAAGCAACATTACGAGACAATTAGTGAAGGCTAGAGAACAAGAGGAAAAGAATCTAGATGCCTATC
AGAAGTTAACTAGTTGGTCAGATTTCTGGTCTTGGTTCGATTTCTCAAAATGGCTTAACATTTTAAAAATGGGAT
TTTTAGTAATAGTAGGAATAATAGGGTTAAGATTACTTTACACAGTATATGGATGTATAGTGAGGGTTAGGCAGG
GATATGTTCCTCTATCTCCACAGATCCATATCCGCGGCAATTTTAAAAGAAAGGGAGGAATAGGGGGACAGACTT
CAGCAGAGAGACTAATTAATATAATAACAACACAATTAGAAATACAACATTTACAAACCAAAATTCAAAAAATTT
TAAATTTTAGAGCCGCGGACCGAGCTCTTACGCGGGTCGAAGCGGAGTACTGTCCTCCGAGTGGAGTACTGTCCT
CCGAGCGGAGTACTGTCCTCCGAGTCGAGGGTCGAAGCGGAGTACTGTCCTCCGAGTGGAGTACTGTCCTCCGAG
CGGAGTACTGTCCTCCGAGTCGACTCTAGAGGGTATATAATGaagcttctgccttctccctcctgtaacgttgag
tttgctagccaccatgtggctgcagaacctgctgttcctgggcattgtggtgtacagcctgtctgcccctacaag
atcccctatcacagtgaccagaccttggaaacatgtggaagccatcaaagaggccctgaatctgctggatgacat
gcctgtgacactgaatgaagaggtggaagtggtgtccaatgagttcagcttcaagaaactgacctgtgtgcagac
caggctgaagatttttgagcagggcctgagaggcaacttcaccaagctgaaaggggctctgaacatgacagccag
ctactaccagacctactgtcctcctacacctgagacagactgtgaaacccaagtgaccacctatgctgacttcat
tgacagcctcaagaccttcctgacagacatcccctttgagtgcaagaaacctggccagaagtgagggcccaccca
gctttcttgtacaaagtggtgataatcgaattcAATCAACCTCTGGATTACAAAATTTGTGAAAGATTGACTGGT
ATTCTTAACTATGTTGCTCCTTTTACGCTATGTGGATACGCTGCTTTAATGCCTTTGTATCATGCTATTGCTTCC
CGTATGGCTTTCATTTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATTGCCACCACCTGTCAG
CTCCTTTCCGGGACTTTCGCTTTCCCCCTCCCTATTGCCACGGCGGAACTCATCGCCGCCTGCCTTGCCCGCTGC
TGGACAGGGGCTCGGCTGTTGGGCACTGACAATTCCGTGGTGTTGTCGGGGAAATCATCGTCCTTTCCTTGGCTG
CTCGCCTGTGTTGCCACCTGGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGCCCTCAGACGAGTCGGATC
TCCCTTTGGGCCGCCTCCCCGCAAGCTTCGCACTTTTTAAAAGAAAAGGGAGGACTGGATGGGATTTATTACTCC
GATAGGACGCTGGCTTGTAACTCAGTCTCTTACTAGGAGACCAGCTTGAGCCTGGGTGTTCGCTGGTTAGCCTAA
CCTGGTTGGCCACCAGGGGTAAGGACTCCTTGGCTTAGAAAGCTAATAAACTTGCCTGCATTAGAGCTCTTACGC
GTCCCGGGCTCGAGATCCGCATCTCAATTAGTCAGCAACCATAGTCCCGCCCCTAACTCCGCCCATCCCGCCCCT
AACTCCGCCCAGTTCCGCCCATTCTCCGCCCCATGGCTGACTAATTTTTTTTATTTATGCAGAGGCCGAGGCCGC
CTCGGCCTCTGAGCTATTCCAGAAGTAGTGAGGAGGCTTTTTTGGAGGCCTAGGCTTTTGCAAAAAGCTAACTTG
TTTATTGCAGCTTATAATGGTTACAAATAAAGCAATAGCATCACAAATTTCACAAATAAAGCATTTTTTTCACTG
CATTCTAGTTGTGGTTTGTCCAAACTCATCAATGTATCTTATCATGTCTGTCCGCTTCCTCGCTCACTGACTCGC
TGCGCTCGGTCGTTCGGCTGCGGCGAGCGGTATCAGCTCACTCAAAGGCGGTAATACGGTTATCCACAGAATCAG
GGGATAACGCAGGAAAGAACATGTGAGCAAAAGGCCAGCAAAAGGCCAGGAACCGTAAAAAGGCCGCGTTGCTGG
CGTTTTTCCATAGGCTCCGCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCATAGCTCACGCTGTAGGTATCTCAGTT
CGGTGTAGGTCGTTCGCTCCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAGCCACTGGTAACAGGATTA
GCAGAGCGAGGTATGTAGGCGGTGCTACAGAGTTCTTGAAGTGGTGGCCTAACTACGGCTACACTAGAAGAACAG
TATTTGGTATCTGCGCTCTGCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTAAAGTATATATGAGTAAA
CTTGGTCTGACAGTTAGAAAAACTCATCGAGCATCAAATGAAACTGCAATTTATTCATATCAGGATTATCAATAC
CATATTTTTGAAAAAGCCGTTTCTGTAATGAAGGAGAAAACTCACCGAGGCAGTTCCATAGGATGGCAAGATCCT
GGTATCGGTCTGCGATTCCGACTCGTCCAACATCAATACAACCTATTAATTTCCCCTCGTCAAAAATAAGGTTAT
CAAGTGAGAAATCACCATGAGTGACGACTGAATCCGGTGAGAATGGCAACAGCTTATGCATTTCTTTCCAGACTT
GTTCAACAGGCCAGCCATTACGCTCGTCATCAAAATCACTCGCATCAACCAAACCGTTATTCATTCGTGATTGCG
CCTGAGCGAGACGAAATACGCGATCGCTGTTAAAAGGACAATTACAAACAGGAATCGAATGCAACCGGCGCAGGA
ACACTGCCAGCGCATCAACAATATTTTCACCTGAATCAGGATATTCTTCTAATACCTGGAATGCTGTTTTTCCGG
GGATCGCAGTGGTGAGTAACCATGCATCATCAGGAGTACGGATAAAATGCTTGATGGTCGGAAGAGGCATAAATT
CCGTCAGCCAGTTTAGTCTGACCATCTCATCTGTAACATCATTGGCAACGCTACCTTTGCCATGTTTCAGAAACA
ACTCTGGCGCATCGGGCTTCCCATACAATCGATAGATTGTCGCACCTGATTGCCCGACATTATCGCGAGCCCATT
TATACCCATATAAATCAGCATCCATGTTGGAATTTAATCGCGGCCTAGAGCAAGACGTTTCCCGTTGAATATGGC
TCATAACACCCCTTGTATTACTGTTTATGTAAGCAGACAGTTTTATTGTTCATGATGATATATTTTTATCTTGTG
CAATGTAACATCAGAGATTTTGAGACACAACAATTGGTCGACGGATCC
SEQ ID NO: 16 pDNA2a plasmid pGM691 (FIG. 2D)

| | |
|---|---|
| attgattatt gactagttat taatagtaat caattacggg gtcattagtt catagcccat | 60 |
| atatggagtt ccgcgttaca taacttacgg taaatggccc gcctggctga ccgcccaacg | 120 |
| acccccgccc attgacgtca ataatgacgt atgttcccat agtaacgcca atagggactt | 180 |
| tccattgacg tcaatgggtg gagtatttac ggtaaactgc ccacttggca gtacatcaag | 240 |
| tgtatcatat gccaagtacg ccccctattg acgtcaatga cggtaaatgg cccgcctggc | 300 |
| attatgccca gtacatgacc ttatgggact ttcctacttg gcagtacatc tacgtattag | 360 |
| tcatcgctat taccatggtc gaggtgagcc ccacgttctg cttcactctc cccatctccc | 420 |
| ccccctcccc acccccaatt ttgtatttat ttatttttta attattttgt gcagcgatgg | 480 |
| gggcgggggg gggggggggg cgcgcgccag gcggggcggg gcggggcgag gggcggggcg | 540 |
| gggcgaggcg gagaggtgcg gcggcagcca atcagagcgg cgcgctccga aagtttcctt | 600 |
| ttatggcgag gcggcggcgg cggcggccct ataaaaagcg aagcgcgcgg cgggcgggag | 660 |
| tcgctgcgcg ctgccttcgc cccgtgcccc gctccgccgc cgcctcgcgc cgcccgcccc | 720 |
| ggctctgact gaccgcgtta ctcccacagg tgagcgggcg ggacggccct tctcctccgg | 780 |
| gctgtaatta gcgcttggtt taatgacggc ttgtttcttt tctgtggctg cgtgaaagcc | 840 |
| ttgaggggct ccgggagggc cctttgtgcg gggggagcgg ctcggggggt gcgtgcgtgt | 900 |
| gtgtgtgcgt ggggagcgcc gcgtgcggct ccgcgctgcc cggcggctgt gagcgctgcg | 960 |
| ggcgcggcgc ggggctttgt gcgctccgca gtgtgcgcga ggggagcgcg gccgggggcg | 1020 |
| gtgccccgcg gtgcgggggg ggctgcgagg ggaacaaagg ctgcgtgcgg ggtgtgtgcg | 1080 |
| tgggggggtg agcagggggt gtgggcgcgt cggtcgggct gcaacccccc ctgcaccccc | 1140 |
| ctccccgagt tgctgagcac ggcccggctt cgggtgcggg gctccgtacg gggcgtggcg | 1200 |
| cggggctcgc cgtgccgggc ggggggtggc ggcaggtggg ggtgccgggc ggggcggggc | 1260 |
| cgcctcgggc cggggagggc tcgggggagg ggcgcggcgg cccccggagc gccggcggct | 1320 |
| gtcgaggcgc ggcgagccgc agccattgcc ttttatggta atcgtgcgag agggcgcagg | 1380 |
| gacttccttt gtcccaaatc tgtgcggagc cgaaatctgg gaggcgccgc cgcaccccct | 1440 |
| ctagcgggcg cggggcgaag cggtgcggcg ccggcaggaa ggaaatgggc ggggagggcc | 1500 |
| ttcgtgcgtc gccgcgccgc cgtccccttc tccctctcca gcctcggggc tgtccgcggg | 1560 |
| gggacggctg ccttcggggg ggacggggca gggcggggtt cggcttctgg cgtgtgaccg | 1620 |
| gcggctctag agcctctgct aaccatgttc atgccttctt ctttttccta cagctcctgg | 1680 |
| gcaacgtgct ggttattgtg ctgtctcatc attttggcaa agaattgctc gagccaccat | 1740 |
| gggagctgcc acatctgccc tgaatagacg gcagctggac cagttcgaga agatcagact | 1800 |
| gcggcccaac ggcaagaaga agtaccagat caagcacctg atctgggccg gcaaagagat | 1860 |
| ggaaagattc ggcctgcacg agcggctgct ggaaaccgag gaaggctgca agagaattat | 1920 |
| cgaggtgctg taccctctgg aacctaccgg ctctgagggc ctgaagtccc tgttcaatct | 1980 |
| cgtgtgcgtg ctgtactgcc tgcacaaaga acagaaagtg aaggacaccg aagaggccgt | 2040 |
| ggccacagtt agacagcact gccacctggt ggaaaaagag aagtccgcca cagagacaag | 2100 |
| cagcggccag aagaagaacg acaagggaat tgctgcccct cctggcggca gccagaattt | 2160 |
| tcctgctcag cagcagggaa acgcctgggt gcacgttcca ctgagcccta gaacactgaa | 2220 |
| tgcctgggtc aaagccgtgg aagagaagaa gtttggcgcc gagatcgtgc ccatgttcca | 2280 |
| ggctctgtct gagggctgca ccccttacga catcaaccag atgctgaacg tgctgggaga | 2340 |
| tcaccagggc gctctgcaga tcgtgaaaga gatcatcaac gaagaggctg cccagtggga | 2400 |
| cgtgacacat ccattgcctg ctggacctct gccagccgga caactgagag atcctagagg | 2460 |
| ctctgatatc gccggcacca ccagctctgt gcaagagcag ctggaatgga tctacaccgc | 2520 |
| caatcctaga gtggacgtgg gcgccatcta cagaagatgg atcatcctgg gcctgcagaa | 2580 |
| atgcgtgaag atgtacaacc ccgtgtccgt gctggacatc agacagggac ccaaagagcc | 2640 |
| cttcaaggac tacgtggacc ggttctataa ggccattaga gccgagcagg ccagcggcga | 2700 |
| agtgaagcag tggatgacag agagcctgct gatccagaac gccaatccag actgcaaagt | 2760 |
| gatcctgaaa ggcctgggca tgcaccccac actggaagag atgctgacag cctgtcaagg | 2820 |
| cgttggcggc ccttcttaca aagccaaagt gatggccgag atgatgcaga ccatgcagaa | 2880 |
| ccagaacatg gtgcagcaag gcggccctaa gagacagagg cctcctctga gatgctacaa | 2940 |
| ctgcggcaag ttcggccaca tgcagagaca gtgtcctgag cctaggaaaa caaaatgtct | 3000 |
| aaagtgtgga aaattgggac acctagcaaa agactgcagg ggacaggtga attttttagg | 3060 |
| gtatggacgg tggatggggg caaaaccgag aaattttccc gccgctactc ttggagcgga | 3120 |
| accgagtgcg cctcctccac cgagcggcac caccccatac gacccagcaa agaagctcct | 3180 |
| gcagcaatat gcagagaaag ggaaacaact gagggagcaa aagaggaatc caccggcaat | 3240 |
| gaatccggat tggaccgagg gatattcttt gaactccctc tttggagaag accaataaag | 3300 |
| accgtgtaca tcgagggcgt gcccatcaag gctctgctgg atacaggcgc cgacgacacc | 3360 |
| atcatcaaag agaacgacct gcagctgagc ggcccttgga ggcctaagat cattggagga | 3420 |
| atcggcggag gcctgaacgt caaagagtac aacgaccggg aagtgaagat cgaggacaag | 3480 |
| atcctgaggg gcacaatcct gctgggcgcc acacctatca acatcatcgg cagaaatctg | 3540 |
| ctggcccctg ccggcgctag actggttatg ggacagctct ctgagaagat ccccgtgaca | 3600 |
| cccgtgaagc tgaaagaagg cgctagagga ccttgtgtgc gacagtggcc tctgagcaaa | 3660 |
| gagaagattg aggccctgca agaaatctgt agccagctgg aacaagaggg caagatcagc | 3720 |
| agagttggcg gcgagaacgc ctacaatacc cctatcttct gcatcaagaa aaaggacaag | 3780 |
| agccagtggc ggatgctggt ggactttaga gagctgaaca aggctaccca ggacttcttc | 3840 |
| gaggtgcagc tgggaattcc tcatcctgcc ggcctgcgga agatgagaca gatcacagtg | 3900 |
| ctggatgtgg gcgacgccta ctacagcatc cctctggacc ccaacttcag aaagtacacc | 3960 |
| gccttcacaa tccccaccgt gaacaatcaa ggccctggca tcagatacca gttcaactgc | 4020 |
| ctgcctcaag gctggaaggg cagccccacc atttttcaga ataccgccgc cagcatcctg | 4080 |
| gaagaaatca agagaaacct gcctgctctg accatcgtgc agtacatgga cgatctgtgg | 4140 |
| gtcggaagcc aagagaatga gcacacccac gacaagctgg tggaacagct gagaacaaag | 4200 |
| ctgcaggcct ggggcctcga aacccctgag aagaaggtgc agaaagaacc tccttacgag | 4260 |
| tggatgggct acaagctgtg gcctcacaag tgggagctga gccggattca gctcgaagag | 4320 |
| aaggacgagt ggaccgtgaa cgacatccag aaactcgtgg gcaagctgaa ttgggcagcc | 4380 |
| cagctgtatc ccggcctgag gaccaagaac atctgcaagc tgatccgggg aaagaagaac | 4440 |
| ctgctggaac tggtcacatg gacacctgag gccgaggccg aatatgccga gaatgccgaa | 4500 |
| atcctgaaaa ccgagcaaga ggggacctac tacaagcctg gcattccaat cagagctgcc | 4560 |
| gtgcagaaac tggaaggcgg ccagtggtcc taccagttta agcaagaagg ccaggtcctg | 4620 |
| aaagtgggca agtacaccaa gcagaagaac acccacacca acgagctgag gacactggct | 4680 |
| ggcctggtcc agaaaatctg caaagaggcc ctggtcattt ggggcatcct gcctgttctg | 4740 |
| gaactgccca ttgagcggga agtgtgggaa cagtggtggg ccgattactg gcaagtgtct | 4800 |
| tggatccccg agtgggactt cgtgtctacc cctcctctgc tgaaactgtg gtacaccctg | 4860 |
| acaaaagagc ccattcctaa agaggacgtc tactacgttg acggcgcctg caaccggaac | 4920 |
| tccaaagaag gcaaggccgg ctacatcagc cagtacggca agcagagagt ggaaaccctg | 4980 |
| gaaaacacca ccaaccagca ggccgagctg accgccatta agatggccct ggaagatagc | 5040 |
| ggccccaatg tgaacatcgt gaccgactct cagtacgcca tgggaatcct gacagcccag | 5100 |
| cctacacaga gcgatagccc tctggttgag cagatcattg ccctgatgat tcagaagcag | 5160 |
| caaatctacc tgcagtgggt gcccgctcac aaaggcatcg gcggaaacga agagatcgat | 5220 |
| aagctggtgt ccaagggaat cagacgggtg ctgttcctgg aaaagattga agaggcccaa | 5280 |
| gaggaacacg agcgctacca caacaactgg aagaatctgg ccgacaccta cggactgccc | 5340 |
| cagatcgtgg ccaaagaaat cgtggctatg tgccccaagt gtcagatcaa gggcgaacct | 5400 |
| gtgcacggcc aagtggatgc ttctcctggc acatggcaga tggactgtac ccacctggaa | 5460 |
| ggcaaagtgg tcatcgtggc tgtgcacgtg gcctccggct ttattgaggc cgaagtgatc | 5520 |
| cccagagaga caggcaaaga aaccgccaag ttcctgctga agatcctgtc cagatggccc | 5580 |
| atcacacagc tgcacaccga caacggccct aacttcacat ctcaagaggt ggccgccatc | 5640 |
| tgttggtggg gaaagattga gcacacaacc ggcattccct acaatccaca gagccagggc | 5700 |
| agcatcgagt ccatgaacaa gcagctcaaa gagattatcg gcaagatccg ggacgactgc | 5760 |
| cagtacacag aaacagccgt gctgatggcc tgtcacatcc acaacttcaa gcggaaaggc | 5820 |
| ggcatcggag gacagacatc tgccgagaga ctgatcaata tcatcaccac tcagctggaa | 5880 |
| atccagcacc tccagaccaa gatccagaag attctgaact tccgggtgta ctaccgcgag | 5940 |
| ggcagagatc ctgtttggaa aggcccagca cagctgatct ggaaaggcga aggtgccgtg | 6000 |
| gtgctgaagg atggctctga tctgaaggtg gtgcccagac ggaaggccaa gattatcaag | 6060 |
| gattacgagc ccaaacagcg cgtgggcaat gaaggcgacg ttgagggcac aagaggcagc | 6120 |
| gacaattgaa attcactcct caggtgcagg ctgcctatca gaaggtggtg gctggtgtgg | 6180 |
| ccaatgccct ggctcacaaa taccactgag atctttttcc ctctgccaaa aattatgggg | 6240 |
| acatcatgaa gccccttgag catctgactt ctggctaata aaggaaattt attttcattg | 6300 |
| caatagtgtg ttggaatttt ttgtgtctct cactcggaag gacatatggg agggcaaatc | 6360 |
| atttaaaaca tcagaatgag tatttggttt agagtttggc aacatatgcc catatgctgg | 6420 |
| ctgccatgaa caaaggttgg ctataaagag gtcatcagta tatgaaacag ccccctgctg | 6480 |
| tccattcctt attccataga aaagccttga cttgaggtta gatttttttt atattttgtt | 6540 |
| ttgtgttatt tttttcttta acatccctaa aattttcctt acatgtttta ctagccagat | 6600 |
| ttttcctcct ctcctgacta ctcccagtca tagctgtccc tcttctctta tggagatccc | 6660 |
| tcgacctgca gcccaagctt ggcgtaatca tggtcatagc tgtttcctgt gtgaaattgt | 6720 |
| tatccgctca caattccaca caacatacga gccggaagca taaagtgtaa agcctggggt | 6780 |
| gcctaatgag tgagctaact cacattaatt gcgttgcgct cactgcccgc tttccagtcg | 6840 |
| ggaaacctgt cgtgccagcg gatccgcatc tcaattagtc agcaaccata gtcccgcccc | 6900 |
| taactccgcc catcccgccc ctaactccgc ccagttccgc ccattctccg ccccatggct | 6960 |
| gactaatttt ttttatttat gcagaggccg aggccgcctc ggcctctgag ctattccaga | 7020 |
| agtagtgagg aggctttttt ggaggcctag gcttttgcaa aaagctaact tgtttattgc | 7080 |
| agcttataat ggttacaaat aaagcaatag catcacaaat ttcacaaata aagcattttt | 7140 |
| ttcactgcat tctagttgtg gtttgtccaa actcatcaat gtatcttatc atgtctgtcc | 7200 |
| gcttcctcgc tcactgactc gctgcgctcg gtcgttcggc tgcggcgagc ggtatcagct | 7260 |
| cactcaaagg cggtaatacg gttatccaca gaatcagggg ataacgcagg aaagaacatg | 7320 |
| tgagcaaaag gccagcaaaa ggccaggaac cgtaaaaagg ccgcgttgct ggcgtttttc | 7380 |
| cataggctcc gcccccctga cgagcatcac aaaaatcgac gctcaagtca gaggtggcga | 7440 |
| aacccgacag gactataaag ataccaggcg tttccccctg gaagctccct cgtgcgctct | 7500 |
| cctgttccga ccctgccgct taccggatac ctgtccgcct ttctcccttc gggaagcgtg | 7560 |
| gcgctttctc atagctcacg ctgtaggtat ctcagttcgg tgtaggtcgt tcgctccaag | 7620 |
| ctgggctgtg tgcacgaacc ccccgttcag cccgaccgct gcgccttatc cggtaactat | 7680 |
| cgtcttgagt ccaacccggt aagacacgac ttatcgccac tggcagcagc cactggtaac | 7740 |
| aggattagca gagcgaggta tgtaggcggt gctacagagt tcttgaagtg gtggcctaac | 7800 |
| tacggctaca ctagaagaac agtatttggt atctgcgctc tgctgaagcc agttaccttc | 7860 |
| ggaaaaagag ttggtagctc ttgatccggc aaacaaacca ccgctggtag cggtggtttt | 7920 |
| tttgtttgca agcagcagat tacgcgcaga aaaaaaggat ctcaagaaga tcctttgatc | 7980 |
| ttttctacgg ggtctgacgc tcagtggaac gaaaactcac gttaagggat tttggtcatg | 8040 |
| agattatcaa aaaggatctt cacctagatc cttttaaatt aaaaatgaag ttttaaatca | 8100 |
| atctaaagta tatatgagta aacttggtct gacagttaga aaaactcatc gagcatcaaa | 8160 |
| tgaaactgca atttattcat atcaggatta tcaataccat atttttgaaa aagccgtttc | 8220 |
| tgtaatgaag gagaaaactc accgaggcag ttccatagga tggcaagatc ctggtatcgg | 8280 |
| tctgcgattc cgactcgtcc aacatcaata caacctatta atttcccctc gtcaaaaata | 8340 |
| aggttatcaa gtgagaaatc accatgagtg acgactgaat ccggtgagaa tggcaacagc | 8400 |
| ttatgcattt ctttccagac ttgttcaaca ggccagccat tacgctcgtc atcaaaatca | 8460 |
| ctcgcatcaa ccaaaccgtt attcattcgt gattgcgcct gagcgagacg aaatacgcga | 8520 |
| tcgctgttaa aaggacaatt acaaacagga atcgaatgca accggcgcag gaacactgcc | 8580 |
| agcgcatcaa caatattttc acctgaatca ggatattctt ctaatacctg gaatgctgtt | 8640 |
| tttccgggga tcgcagtggt gagtaaccat gcatcatcag gagtacggat aaaatgcttg | 8700 |
| atggtcggaa gaggcataaa ttccgtcagc cagtttagtc tgaccatctc atctgtaaca | 8760 |
| tcattggcaa cgctaccttt gccatgtttc agaaacaact ctggcgcatc gggcttccca | 8820 |
| tacaatcgat agattgtcgc acctgattgc ccgacattat cgcgagccca tttataccca | 8880 |
| tataaatcag catccatgtt ggaatttaat cgcggcctag agcaagacgt ttcccgttga | 8940 |
| atatggctca taacacccct tgtattactg tttatgtaag cagacagttt tattgttcat | 9000 |
| gatgatatat ttttatcttg tgcaatgtaa catcagagat tttgagacac aacaattggt | 9060 |
| cgac | 9064 |

SEQ ID NO: 17 pDNA2a plasmid pGM297 (FIG. 2E)

| | |
|---|---|
| attgattatt gactagttat taatagtaat caattacggg gtcattagtt catagcccat | 60 |
| atatggagtt ccgcgttaca taacttacgg taaatggccc gcctggctga ccgcccaacg | 120 |
| acccccgccc attgacgtca ataatgacgt atgttcccat agtaacgcca atagggactt | 180 |
| tccattgacg tcaatgggtg gagtatttac ggtaaactgc ccacttggca gtacatcaag | 240 |
| tgtatcatat gccaagtacg ccccctattg acgtcaatga cggtaaatgg cccgcctggc | 300 |
| attatgccca gtacatgacc ttatgggact ttcctacttg gcagtacatc tacgtattag | 360 |
| tcatcgctat taccatggtc gaggtgagcc ccacgttctg cttcactctc cccatctccc | 420 |
| ccccctcccc acccccaatt ttgtatttat ttatttttta attattttgt gcagcgatgg | 480 |
| gggcgggggg gggggggggg cgcgcgccag gcggggcggg gcggggcgag gggcggggcg | 540 |
| gggcgaggcg gagaggtgcg gcggcagcca atcagagcgg cgcgctccga aagtttcctt | 600 |
| ttatggcgag gcggcggcgg cggcggccct ataaaaagcg aagcgcgcgg cgggcgggag | 660 |
| tcgctgcgcg ctgccttcgc cccgtgcccc gctccgccgc cgcctcgcgc cgcccgcccc | 720 |
| ggctctgact gaccgcgtta ctcccacagg tgagcgggcg ggacggccct tctcctccgg | 780 |
| gctgtaatta gcgcttggtt taatgacggc ttgtttcttt tctgtggctg cgtgaaagcc | 840 |
| ttgaggggct ccgggagggc cctttgtgcg gggggagcgg ctcggggggt gcgtgcgtgt | 900 |
| gtgtgtgcgt ggggagcgcc gcgtgcggct ccgcgctgcc cggcggctgt gagcgctgcg | 960 |
| ggcgcggcgc ggggctttgt gcgctccgca gtgtgcgcga ggggagcgcg gccgggggcg | 1020 |
| gtgccccgcg gtgcgggggg ggctgcgagg ggaacaaagg ctgcgtgcgg ggtgtgtgcg | 1080 |
| tgggggggtg agcagggggt gtgggcgcgt cggtcgggct gcaacccccc ctgcaccccc | 1140 |
| ctccccgagt tgctgagcac ggcccggctt cgggtgcggg gctccgtacg gggcgtggcg | 1200 |
| cggggctcgc cgtgccgggc ggggggtggc ggcaggtggg ggtgccgggc ggggcggggc | 1260 |
| cgcctcgggc cggggagggc tcgggggagg ggcgcggcgg cccccggagc gccggcggct | 1320 |
| gtcgaggcgc ggcgagccgc agccattgcc ttttatggta atcgtgcgag agggcgcagg | 1380 |
| gacttccttt gtcccaaatc tgtgcggagc cgaaatctgg gaggcgccgc cgcaccccct | 1440 |
| ctagcgggcg cggggcgaag cggtgcggcg ccggcaggaa ggaaatgggc ggggagggcc | 1500 |
| ttcgtgcgtc gccgcgccgc cgtccccttc tccctctcca gcctcggggc tgtccgcggg | 1560 |
| gggacggctg ccttcggggg ggacggggca gggcggggtt cggcttctgg cgtgtgaccg | 1620 |
| gcggctctag agcctctgct aaccatgttc atgccttctt ctttttccta cagctcctgg | 1680 |
| gcaacgtgct ggttattgtg ctgtctcatc attttggcaa agaattgctc gagactagtg | 1740 |
| acttggtgag taggcttcga gcctagttag aggactagga gaggccgtag ccgtaactac | 1800 |
| tctgggcaag tagggcaggc ggtgggtacg caatgggggc ggctacctca gcactaaata | 1860 |
| ggagacaatt agaccaattt gagaaaatac gacttcgccc gaacggaaag aaaaagtacc | 1920 |
| aaattaaaca tttaatatgg gcaggcaagg agatggagcg cttcggcctc catgagaggt | 1980 |
| tgttggagac agaggagggg tgtaaaagaa tcatagaagt cctctacccc ctagaaccaa | 2040 |
| caggatcgga gggcttaaaa agtctgttca atcttgtgtg cgtactatat tgcttgcaca | 2100 |
| aggaacagaa agtgaaagac acagaggaag cagtagcaac agtaagacaa cactgccatc | 2160 |
| tagtggaaaa agaaaaaagt gcaacagaga catctagtgg acaaaagaaa aatgacaagg | 2220 |
| gaatagcagc gccacctggt ggcagtcaga attttccagc gcaacaacaa ggaaatgcct | 2280 |
| gggtacatgt acccttgtca ccgcgcacct taaatgcgtg ggtaaaagca gtagaggaga | 2340 |
| aaaaatttgg agcagaaata gtacccatgt ttcaagccct atcagaaggc tgcacaccct | 2400 |
| atgacattaa tcagatgctt aatgtgctag gagatcatca aggggcatta caaatagtga | 2460 |
| aagagatcat taatgaagaa gcagcccagt gggatgtaac acacccacta cccgcaggac | 2520 |
| ccctaccagc aggacagctc agggaccctc gcggctcaga tatagcaggg accaccagct | 2580 |
| cagtacaaga acagttagaa tggatctata ctgctaaccc ccgggtagat gtaggtgcca | 2640 |
| tctaccggag atggattatt ctaggacttc aaaagtgtgt caaaatgtac aacccagtat | 2700 |
| cagtcctaga cattaggcag ggacctaaag agcccttcaa ggattatgtg gacagatttt | 2760 |
| acaaggcaat tagagcagaa caagcctcag gggaagtgaa acaatggatg acagaatcat | 2820 |
| tactcattca aaatgctaat ccagattgta aggtcatcct gaagggccta ggaatgcacc | 2880 |
| ccacccttga agaaatgtta acggcttgtc agggggtagg aggcccaagc tacaaagcaa | 2940 |
| aagtaatggc agaaatgatg cagaccatgc aaaatcaaaa catggtgcag cagggaggtc | 3000 |
| caaaaagaca aagaccccca ctaagatgtt ataattgtgg aaaatttggc catatgcaaa | 3060 |
| gacaatgtcc ggaaccaagg aaaacaaaat gtctaaagtg tggaaaattg ggacacctag | 3120 |
| caaaagactg caggggacag gtgaattttt tagggtatgg acggtggatg ggggcaaaac | 3180 |
| cgagaaattt tcccgccgct actcttggag cggaaccgag tgcgcctcct ccaccgagcg | 3240 |
| gcaccacccc atacgaccca gcaaagaagc tcctgcagca atatgcagag aaagggaaac | 3300 |
| aactgaggga gcaaaagagg aatccaccgg caatgaatcc ggattggacc gagggatatt | 3360 |
| ctttgaactc cctctttgga gaagaccaat aaagacagtg tatatagaag gggtccccat | 3420 |
| taaggcactg ctagacacag gggcagatga caccataatt aaagaaaatg atttacaatt | 3480 |
| atcaggtcca tggagaccca aaattatagg gggcatagga ggaggcctta atgtaaaaga | 3540 |
| atataacgac agggaagtaa aaatagaaga taaaattttg agaggaacaa tattgttagg | 3600 |
| agcaactccc attaatataa taggtagaaa tttgctggcc ccggcaggtg cccggttagt | 3660 |
| aatgggacaa ttatcagaaa aaattcctgt cacacctgtc aaattgaagg aaggggctcg | 3720 |
| gggaccctgt gtaagacaat ggcctctctc taaagagaag attgaagctt tacaggaaat | 3780 |
| atgttcccaa ttagagcagg aaggaaaaat cagtagagta ggaggagaaa atgcatacaa | 3840 |
| taccccaata ttttgcataa agaagaagga caaatcccag tggaggatgc tagtagactt | 3900 |
| tagagagtta aataaggcaa cccaagattt ctttgaagtg caattaggga taccccaccc | 3960 |
| agcaggatta agaaagatga gacagataac agttttagat gtaggagacg cctattattc | 4020 |
| cataccattg gatccaaatt ttaggaaata tactgctttt actattccca cagtgaataa | 4080 |
| tcagggaccc gggattaggt atcaattcaa ctgtctcccg caagggtgga aaggatctcc | 4140 |
| tacaatcttc caaaatacag cagcatccat tttggaggag ataaaaagaa acttgccagc | 4200 |
| actaaccatt gtacaataca tggatgattt atgggtaggt tctcaagaaa atgaacacac | 4260 |
| ccatgacaaa ttagtagaac agttaagaac aaaattacaa gcctggggct tagaaacccc | 4320 |
| agaaaagaag gtgcaaaaag aaccacctta tgagtggatg ggatacaaac tttggcctca | 4380 |
| caaatgggaa ctaagcagaa tacaactgga ggaaaaagat gaatggactg tcaatgacat | 4440 |
| ccagaagtta gttgggaaac taaattgggc agcacaattg tatccaggtc ttaggaccaa | 4500 |
| gaatatatgc aagttaatta gaggaaagaa aaatctgtta gagctagtga cttggacacc | 4560 |
| tgaggcagaa gctgaatatg cagaaaatgc agagattctt aaaacagaac aggaaggaac | 4620 |
| ctattacaaa ccaggaatac ctattagggc agcagtacag aaattggaag gaggacagtg | 4680 |
| gagttaccaa ttcaaacaag aaggacaagt cttgaaagta ggaaaataca ccaagcaaaa | 4740 |
| gaacacccat acaaatgaac ttcgcacatt agctggttta gtgcagaaga tttgcaaaga | 4800 |
| agctctagtt atttggggga tattaccagt tctagaactc ccgatagaaa gagaggtatg | 4860 |
| ggaacaatgg tgggcggatt actggcaggt aagctggatt cccgaatggg attttgtcag | 4920 |
| caccccacct ttgctcaaac tatggtacac attaacaaaa gaacccatac ccaaggagga | 4980 |
| cgtttactat gtagatggag catgcaacag aaattcaaaa gaaggaaaag caggatacat | 5040 |
| ctcacaatac ggaaaacaga gagtagaaac attagaaaac actaccaatc agcaagcaga | 5100 |
| attaacagct ataaaaatgg ctttggaaga cagtgggcct aatgtgaaca tagtaacaga | 5160 |
| ctctcaatat gcaatgggaa ttttgacagc acaacccaca caaagtgatt caccattagt | 5220 |
| agagcaaatt atagccttaa tgatacaaaa gcaacaaata tatttgcagt gggtaccagc | 5280 |
| acataaagga ataggaggaa atgaggagat agataaatta gtgagtaaag gcattagaag | 5340 |
| agttttattc ttagaaaaaa tagaagaagc tcaagaagag catgaaagat atcataataa | 5400 |
| ttggaaaaac ctagcagata catatgggct tccacaaata gtagcaaaag agatagtggc | 5460 |
| catgtgtcca aaatgtcaga taaagggaga accagtgcat ggacaagtgg atgcctcacc | 5520 |
| tggaacatgg cagatggatt gtactcatct agaaggaaaa gtagtcatag ttgcggtcca | 5580 |
| tgtagccagt ggattcatag aagcagaagt catacctagg gaaacaggaa aagaaacggc | 5640 |
| aaagtttcta ttaaaaatac tgagtagatg gcctataaca cagttacaca cagacaatgg | 5700 |
| gcctaacttt acctcccaag aagtggcagc aatatgttgg tggggaaaaa ttgaacatac | 5760 |
| aacaggtata ccatataacc cccaatctca aggatcaata gaaagcatga acaaacaatt | 5820 |
| aaaagagata attgggaaaa taagagatga ttgccaatat acagagacag cagtactgat | 5880 |
| ggcttgccat attcacaatt ttaaaagaaa gggaggaata gggggacaga cttcagcaga | 5940 |
| gagactaatt aatataataa caacacaatt agaaatacaa catttacaaa ccaaaattca | 6000 |
| aaaaatttta aattttagag tctactacag agaagggaga gaccctgtgt ggaaaggacc | 6060 |
| agcacaatta atctggaaag gggaaggagc agtggtcctc aaggacggaa gtgacctaaa | 6120 |
| ggttgtacca agaaggaaag ctaaaattat taaggattat gaacccaaac aaagagtggg | 6180 |
| taatgagggt gacgtggaag gtaccagggg atctgataac taaatggcag ggaatagtca | 6240 |
| gatattggat gagacaaaga aatttgaaat ggaactatta tatgcatcag ctggcggccg | 6300 |
| cgaattcact agtgattccc gtttgtgcta gggttcttag gcttcttggg ggctgctgga | 6360 |
| actgcaatgg gagcagcggc gacagccctg acggtccagt ctcagcattt gcttgctggg | 6420 |
| atactgcagc agcagaagaa tctgctggcg gctgtggagg ctcaacagca gatgttgaag | 6480 |
| ctgaccattt ggggtgttaa aaacctcaat gcccgcgtca cagcccttga gaagtaccta | 6540 |
| gaggatcagg cacgactaaa ctcctggggg tgcgcatgga aacaagtatg tcataccaca | 6600 |
| gtggagtggc cctggacaaa tcggactccg gattggcaaa atatgacttg gttggagtgg | 6660 |
| gaaagacaaa tagctgattt ggaaagcaac attacgagac aattagtgaa ggctagagaa | 6720 |
| caagaggaaa agaatctaga tgcctatcag aagttaacta gttggtcaga tttctggtct | 6780 |
| tggttcgatt tctcaaaatg gcttaacatt ttaaaaatgg gatttttagt aatagtagga | 6840 |
| ataatagggt taagattact ttacacagta tatggatgta tagtgagggt taggcaggga | 6900 |
| tatgttcctc tatctccaca gatccatatc caatcgaatt cccgcggccg caattcactc | 6960 |
| ctcaggtgca ggctgcctat cagaaggtgg tggctggtgt ggccaatgcc ctggctcaca | 7020 |
| aataccactg agatcttttt ccctctgcca aaaattatgg ggacatcatg aagccccttg | 7080 |
| agcatctgac ttctggctaa taaaggaaat ttattttcat tgcaatagtg tgttggaatt | 7140 |
| ttttgtgtct ctcactcgga aggacatatg ggagggcaaa tcatttaaaa catcagaatg | 7200 |
| agtatttggt ttagagtttg gcaacatatg cccatatgct ggctgccatg aacaaaggtt | 7260 |
| ggctataaag aggtcatcag tatatgaaac agccccctgc tgtccattcc ttattccata | 7320 |
| gaaaagcctt gacttgaggt tagatttttt ttatattttg ttttgtgtta tttttttctt | 7380 |
| taacatccct aaaattttcc ttacatgttt tactagccag atttttcctc ctctcctgac | 7440 |
| tactcccagt catagctgtc cctcttctct tatggagatc cctcgacctg cagcccaagc | 7500 |
| ttggcgtaat catggtcata gctgtttcct gtgtgaaatt gttatccgct cacaattcca | 7560 |
| cacaacatac gagccggaag cataaagtgt aaagcctggg gtgcctaatg agtgagctaa | 7620 |
| ctcacattaa ttgcgttgcg ctcactgccc gctttccagt cgggaaacct gtcgtgccag | 7680 |
| cggatccgca tctcaattag tcagcaacca tagtcccgcc cctaactccg cccatcccgc | 7740 |
| ccctaactcc gcccagttcc gcccattctc cgccccatgg ctgactaatt ttttttattt | 7800 |
| atgcagaggc cgaggccgcc tcggcctctg agctattcca gaagtagtga ggaggctttt | 7860 |
| ttggaggcct aggcttttgc aaaaagctaa cttgtttatt gcagcttata atggttacaa | 7920 |
| ataaagcaat agcatcacaa atttcacaaa taaagcattt ttttcactgc attctagttg | 7980 |
| tggtttgtcc aaactcatca atgtatctta tcatgtctgt ccgcttcctc gctcactgac | 8040 |
| tcgctgcgct cggtcgttcg gctgcggcga gcggtatcag ctcactcaaa ggcggtaata | 8100 |
| cggttatcca cagaatcagg ggataacgca ggaaagaaca tgtgagcaaa aggccagcaa | 8160 |
| aaggccagga accgtaaaaa ggccgcgttg ctggcgtttt tccataggct ccgcccccct | 8220 |
| gacgagcatc acaaaaatcg acgctcaagt cagaggtggc gaaacccgac aggactataa | 8280 |
| agataccagg cgtttccccc tggaagctcc ctcgtgcgct ctcctgttcc gaccctgccg | 8340 |
| cttaccggat acctgtccgc ctttctccct tcgggaagcg tggcgctttc tcatagctca | 8400 |
| cgctgtaggt atctcagttc ggtgtaggtc gttcgctcca agctgggctg tgtgcacgaa | 8460 |
| ccccccgttc agcccgaccg ctgcgcctta tccggtaact atcgtcttga gtccaacccg | 8520 |
| gtaagacacg acttatcgcc actggcagca gccactggta acaggattag cagagcgagg | 8580 |
| tatgtaggcg gtgctacaga gttcttgaag tggtggccta actacggcta cactagaaga | 8640 |
| acagtatttg gtatctgcgc tctgctgaag ccagttacct tcggaaaaag agttggtagc | 8700 |
| tcttgatccg gcaaacaaac caccgctggt agcggtggtt tttttgtttg caagcagcag | 8760 |
| attacgcgca gaaaaaaagg atctcaagaa gatcctttga tcttttctac ggggtctgac | 8820 |
| gctcagtgga acgaaaactc acgttaaggg attttggtca tgagattatc aaaaaggatc | 8880 |
| ttcacctaga tccttttaaa ttaaaaatga agttttaaat caatctaaag tatatatgag | 8940 |
| taaacttggt ctgacagtta gaaaaactca tcgagcatca aatgaaactg caatttattc | 9000 |
| atatcaggat tatcaatacc atatttttga aaaagccgtt tctgtaatga aggagaaaac | 9060 |
| tcaccgaggc agttccatag gatggcaaga tcctggtatc ggtctgcgat tccgactcgt | 9120 |
| ccaacatcaa tacaacctat taatttcccc tcgtcaaaaa taaggttatc aagtgagaaa | 9180 |
| tcaccatgag tgacgactga atccggtgag aatggcaaca gcttatgcat ttctttccag | 9240 |
| acttgttcaa caggccagcc attacgctcg tcatcaaaat cactcgcatc aaccaaaccg | 9300 |
| ttattcattc gtgattgcgc ctgagcgaga cgaaatacgc gatcgctgtt aaaaggacaa | 9360 |
| ttacaaacag gaatcgaatg caaccggcgc aggaacactg ccagcgcatc aacaatattt | 9420 |
| tcacctgaat caggatattc ttctaatacc tggaatgctg tttttccggg gatcgcagtg | 9480 |
| gtgagtaacc atgcatcatc aggagtacgg ataaaatgct tgatggtcgg aagaggcata | 9540 |
| aattccgtca gccagtttag tctgaccatc tcatctgtaa catcattggc aacgctacct | 9600 |
| ttgccatgtt tcagaaacaa ctctggcgca tcgggcttcc catacaatcg atagattgtc | 9660 |
| gcacctgatt gcccgacatt atcgcgagcc catttatacc catataaatc agcatccatg | 9720 |
| ttggaattta atcgcggcct agagcaagac gtttcccgtt gaatatggct cataacaccc | 9780 |
| cttgtattac tgtttatgta agcagacagt tttattgttc atgatgatat atttttatct | 9840 |
| tgtgcaatgt aacatcagag attttgagac acaacaattg gtcgac | 9886 |

SEQ ID NO: 18 pDNA2b plasmid pGM299 (FIG. 2F)

| | |
|---|---|
| tcaatattgg ccattagcca tattattcat tggttatata gcataaatca atattggcta | 60 |
| ttggccattg catacgttgt atctatatca taatatgtac atttatattg gctcatgtcc | 120 |
| aatatgaccg ccatgttggc attgattatt gactagttat taatagtaat caattacggg | 180 |
| gtcattagtt catagcccat atatggagtt ccgcgttaca taacttacgg taaatggccc | 240 |
| gcctggctga ccgcccaacg acccccgccc attgacgtca ataatgacgt atgttcccat | 300 |
| agtaacgcca atagggactt tccattgacg tcaatgggtg gagtatttac ggtaaactgc | 360 |
| ccacttggca gtacatcaag tgtatcatat gccaagtccg ccccctattg acgtcaatga | 420 |
| cggtaaatgg cccgcctggc attatgccca gtacatgacc ttacgggact ttcctacttg | 480 |
| gcagtacatc tacgtattag tcatcgctat taccatggtg atgcggtttt ggcagtacac | 540 |
| caatgggcgt ggatagcggt ttgactcacg gggatttcca agtctccacc ccattgacgt | 600 |
| caatgggagt ttgttttggc accaaaatca acgggacttt ccaaaatgtc gtaataaccc | 660 |
| cgccccgttg acgcaaatgg gcggtaggcg tgtacggtgg gaggtctata taagcagagc | 720 |
| tcgtttagtg aaccgtcaga tcactagaag ctttattgcg gtagtttatc acagttaaat | 780 |
| tgctaacgca gtcagtgctt ctgacacaac agtctcgaac ttaagctgca gaagttggtc | 840 |
| gtgaggcact gggcaggtaa gtatcaaggt tacaagacag gtttaaggag accaatagaa | 900 |
| actgggcttg tcgagacaga gaagactctt gcgtttctga taggcaccta ttggtcttac | 960 |
| tgacatccac tttgcctttc tctccacagg tgtccactcc cagttcaatt acagctctta | 1020 |
| aggctagagt acttaatacg actcactata ggctagcctc gagaattcga ttatgcccct | 1080 |
| aggaccagaa gaaagaagat tgcttcgctt gatttggctc ctttacagca ccaatccata | 1140 |
| tccaccaagt ggggaaggga cggccagaca acgccgacga gccaggagaa ggtggagaca | 1200 |
| acagcaggat caaattagag tcttggtaga aagactccaa gagcaggtgt atgcagttga | 1260 |
| ccgcctggct gacgaggctc aacacttggc tatacaacag ttgcctgacc ctcctcattc | 1320 |
| agcttagaat cactagtgaa ttcacgcgtg gtacctctag agtcgacccg ggcggccgct | 1380 |
| tcgagcagac atgataagat acattgatga gtttggacaa accacaacta gaatgcagtg | 1440 |
| aaaaaaatgc tttatttgtg aaatttgtga tgctattgct ttatttgtaa ccattataag | 1500 |
| ctgcaataaa caagttaaca acaacaattg cattcatttt atgtttcagg ttcaggggga | 1560 |
| gatgtgggag gttttttaaa gcaagtaaaa cctctacaaa tgtggtaaaa tcgataagga | 1620 |
| tccgtcgacc aattgttgtg tctcaaaatc tctgatgtta cattgcacaa gataaaaata | 1680 |
| tatcatcatg aacaataaaa ctgtctgctt acataaacag taatacaagg ggtgttatga | 1740 |
| gccatattca acgggaaacg tcttgctcta ggccgcgatt aaattccaac atggatgctg | 1800 |
| atttatatgg gtataaatgg gctcgcgata atgtcgggca atcaggtgcg acaatctatc | 1860 |
| gattgtatgg gaagcccgat gcgccagagt tgtttctgaa acatggcaaa ggtagcgttg | 1920 |
| ccaatgatgt tacagatgag atggtcagac taaactggct gacggaattt atgcctcttc | 1980 |
| cgaccatcaa gcattttatc cgtactcctg atgatgcatg gttactcacc actgcgatcc | 2040 |
| ccggaaaaac agcattccag gtattagaag aatatcctga ttcaggtgaa aatattgttg | 2100 |
| atgcgctggc agtgttcctg cgccggttgc attcgattcc tgtttgtaat tgtcctttta | 2160 |
| acagcgatcg cgtatttcgt ctcgctcagg cgcaatcacg aatgaataac ggtttggttg | 2220 |
| atgcgagtga ttttgatgac gagcgtaatg gctggcctgt tgaacaagtc tggaaagaaa | 2280 |
| tgcataagct gttgccattc tcaccggatt cagtcgtcac tcatggtgat ttctcacttg | 2340 |
| ataaccttat ttttgacgag gggaaattaa taggttgtat tgatgttgga cgagtcggaa | 2400 |
| tcgcagaccg ataccaggat cttgccatcc tatggaactg cctcggtgag ttttctcctt | 2460 |
| cattacagaa acggcttttt caaaaatatg gtattgataa tcctgatatg aataaattgc | 2520 |
| agtttcattt gatgctcgat gagtttttct aactgtcaga ccaagtttac tcatatatac | 2580 |
| tttagattga tttaaaactt catttttaat ttaaaaggat ctaggtgaag atcctttttg | 2640 |
| ataatctcat gaccaaaatc ccttaacgtg agttttcgtt ccactgagcg tcagaccccg | 2700 |
| tagaaaagat caaaggatct tcttgagatc ctttttttct gcgcgtaatc tgctgcttgc | 2760 |
| aaacaaaaaa accaccgcta ccagcggtgg tttgtttgcc ggatcaagag ctaccaactc | 2820 |
| tttttccgaa ggtaactggc ttcagcagag cgcagatacc aaatactgtt cttctagtgt | 2880 |
| agccgtagtt aggccaccac ttcaagaact ctgtagcacc gcctacatac ctcgctctgc | 2940 |
| taatcctgtt accagtggct gctgccagtg gcgataagtc gtgtcttacc gggttggact | 3000 |
| caagacgata gttaccggat aaggcgcagc ggtcgggctg aacggggggt tcgtgcacac | 3060 |
| agcccagctt ggagcgaacg acctacaccg aactgagata cctacagcgt gagctatgag | 3120 |
| aaagcgccac gcttcccgaa gggagaaagg cggacaggta tccggtaagc ggcagggtcg | 3180 |
| gaacaggaga gcgcacgagg gagcttccag ggggaaacgc ctggtatctt tatagtcctg | 3240 |
| tcgggtttcg ccacctctga cttgagcgtc gatttttgtg atgctcgtca ggggggcgga | 3300 |
| gcctatggaa aaacgccagc aacgcggcct ttttacggtt cctggccttt tgctggcctt | 3360 |
| ttgctcacat ggctcgacag atct | 3384 |

SEQ ID NO: 19 pDNA3a plasmid pGM301 (FIG. 2G)

| | |
|---|---|
| attgattatt gactagttat taatagtaat caattacggg gtcattagtt catagcccat | 60 |
| atatggagtt ccgcgttaca taacttacgg taaatggccc gcctggctga ccgcccaacg | 120 |
| acccccgccc attgacgtca ataatgacgt atgttcccat agtaacgcca atagggactt | 180 |
| tccattgacg tcaatgggtg gagtatttac ggtaaactgc ccacttggca gtacatcaag | 240 |
| tgtatcatat gccaagtacg ccccctattg acgtcaatga cggtaaatgg cccgcctggc | 300 |
| attatgccca gtacatgacc ttatgggact ttcctacttg gcagtacatc tacgtattag | 360 |
| tcatcgctat taccatggtc gaggtgagcc ccacgttctg cttcactctc cccatctccc | 420 |
| ccccctcccc acccccaatt ttgtatttat ttatttttta attattttgt gcagcgatgg | 480 |
| gggcgggggg gggggggggg cgcgcgccag gcggggcggg gcggggcgag gggcggggcg | 540 |
| gggcgaggcg gagaggtgcg gcggcagcca atcagagcgg cgcgctccga aagtttcctt | 600 |
| ttatggcgag gcggcggcgg cggcggccct ataaaaagcg aagcgcgcgg cgggcgggag | 660 |
| tcgctgcgcg ctgccttcgc cccgtgcccc gctccgccgc cgcctcgcgc cgcccgcccc | 720 |
| ggctctgact gaccgcgtta ctcccacagg tgagcgggcg ggacggccct tctcctccgg | 780 |
| gctgtaatta gcgcttggtt taatgacggc ttgtttcttt tctgtggctg cgtgaaagcc | 840 |
| ttgaggggct ccgggagggc cctttgtgcg gggggagcgg ctcggggggt gcgtgcgtgt | 900 |
| gtgtgtgcgt ggggagcgcc gcgtgcggct ccgcgctgcc cggcggctgt gagcgctgcg | 960 |
| ggcgcggcgc ggggctttgt gcgctccgca gtgtgcgcga ggggagcgcg gccgggggcg | 1020 |
| gtgccccgcg gtgcgggggg ggctgcgagg ggaacaaagg ctgcgtgcgg ggtgtgtgcg | 1080 |
| tgggggggtg agcagggggt gtgggcgcgt cggtcgggct gcaacccccc ctgcaccccc | 1140 |
| ctccccgagt tgctgagcac ggcccggctt cgggtgcggg gctccgtacg gggcgtggcg | 1200 |
| cggggctcgc cgtgccgggc ggggggtggc ggcaggtggg ggtgccgggc ggggcggggc | 1260 |
| cgcctcgggc cggggagggc tcgggggagg ggcgcggcgg cccccggagc gccggcggct | 1320 |
| gtcgaggcgc ggcgagccgc agccattgcc ttttatggta atcgtgcgag agggcgcagg | 1380 |
| gacttccttt gtcccaaatc tgtgcggagc cgaaatctgg gaggcgccgc cgcaccccct | 1440 |
| ctagcgggcg cggggcgaag cggtgcggcg ccggcaggaa ggaaatgggc ggggagggcc | 1500 |
| ttcgtgcgtc gccgcgccgc cgtccccttc tccctctcca gcctcggggc tgtccgcggg | 1560 |
| gggacggctg ccttcggggg ggacggggca gggcggggtt cggcttctgg cgtgtgaccg | 1620 |
| gcggctctag agcctctgct aaccatgttc atgccttctt ctttttccta cagctcctgg | 1680 |
| gcaacgtgct ggttattgtg ctgtctcatc attttggcaa agaattcgat tgccatggca | 1740 |
| acatatatcc agagagtaca gtgcatctca acatcactac tggttgttct caccacattg | 1800 |
| gtctcgtgtc agattcccag ggataggctc tctaacatag gggtcatagt cgatgaaggg | 1860 |
| aaatcactga agatagctgg atcccacgaa tcgaggtaca tagtactgag tctagttccg | 1920 |
| ggggtagact ttgagaatgg gtgcggaaca gcccaggtta tccagtacaa gagcctactg | 1980 |
| aacaggctgt taatcccatt gagggatgcc ttagatcttc aggaggctct gataactgtc | 2040 |
| accaatgata cgacacaaaa tgccggtgct ccccagtcga gattcttcgg tgctgtgatt | 2100 |
| ggtactatcg cacttggagt ggcgacatca gcacaaatca ccgcagggat tgcactagcc | 2160 |
| gaagcgaggg aggccaaaag agacatagcg ctcatcaaag aatcgatgac aaaaacacac | 2220 |
| aagtctatag aactgctgca aaacgctgtg ggggaacaaa ttcttgctct aaagacactc | 2280 |
| caggatttcg tgaatgatga gatcaaaccc gcaataagcg aattaggctg tgagactgct | 2340 |
| gccttaagac tgggtataaa attgacacag cattactccg agctgttaac tgcgttcggc | 2400 |
| tcgaatttcg gaaccatcgg agagaagagc ctcacgctgc aggcgctgtc ttcactttac | 2460 |
| tctgctaaca ttactgagat tatgaccaca atcaggacag ggcagtctaa catctatgat | 2520 |
| gtcatttata cagaacagat caaaggaacg gtgatagatg tggatctaga gagatacatg | 2580 |
| gtcaccctgt ctgtgaagat ccctattctt tctgaagtcc caggtgtgct catacacaag | 2640 |
| gcatcatcta tttcttacaa catagacggg gaggaatggt atgtgactgt ccccagccat | 2700 |
| atactcagtc gtgcttcttt cttagggggt gcagacataa ccgattgtgt tgagtccaga | 2760 |
| ttgacctata tatgccccag ggatcccgca caactgatac ctgacagcca gcaaaagtgt | 2820 |
| atcctggggg acacaacaag gtgtcctgtc acaaaagttg tggacagcct tatccccaag | 2880 |
| tttgcttttg tgaatggggg cgttgttgct aactgcatag catccacatg tacctgcggg | 2940 |
| acaggccgaa gaccaatcag tcaggatcgc tctaaaggtg tagtattcct aacccatgac | 3000 |
| aactgtggtc ttataggtgt caatggggta gaattgtatg ctaaccggag agggcacgat | 3060 |
| gccacttggg gggtccagaa cttgacagtc ggtcctgcaa ttgctatcag acccgttgat | 3120 |
| atttctctca accttgctga tgctacgaat ttcttgcaag actctaaggc tgagcttgag | 3180 |
| aaagcacgga aaatcctctc ggaggtaggt agatggtaca actcaagaga gactgtgatt | 3240 |
| acgatcatag tagttatggt cgtaatattg gtggtcatta tagtgatcat catcgtgctt | 3300 |
| tatagactca gaaggtgaaa tcactagtga attcactcct caggtgcagg ctgcctatca | 3360 |
| gaaggtggtg gctggtgtgg ccaatgccct ggctcacaaa taccactgag atctttttcc | 3420 |
| ctctgccaaa aattatgggg acatcatgaa gccccttgag catctgactt ctggctaata | 3480 |
| aaggaaattt attttcattg caatagtgtg ttggaatttt ttgtgtctct cactcggaag | 3540 |
| gacatatggg agggcaaatc atttaaaaca tcagaatgag tatttggttt agagtttggc | 3600 |
| aacatatgcc catatgctgg ctgccatgaa caaaggttgg ctataaagag gtcatcagta | 3660 |
| tatgaaacag ccccctgctg tccattcctt attccataga aaagccttga cttgaggtta | 3720 |
| gatttttttt atattttgtt ttgtgttatt tttttcttta acatccctaa aattttcctt | 3780 |
| acatgtttta ctagccagat ttttcctcct ctcctgacta ctcccagtca tagctgtccc | 3840 |
| tcttctctta tggagatccc tcgacctgca gcccaagctt ggcgtaatca tggtcatagc | 3900 |
| tgtttcctgt gtgaaattgt tatccgctca caattccaca caacatacga gccggaagca | 3960 |
| taaagtgtaa agcctggggt gcctaatgag tgagctaact cacattaatt gcgttgcgct | 4020 |
| cactgcccgc tttccagtcg ggaaacctgt cgtgccagcg gatccgcatc tcaattagtc | 4080 |
| agcaaccata gtcccgcccc taactccgcc catcccgccc ctaactccgc ccagttccgc | 4140 |
| ccattctccg ccccatggct gactaatttt ttttatttat gcagaggccg aggccgcctc | 4200 |
| ggcctctgag ctattccaga agtagtgagg aggctttttt ggaggcctag gcttttgcaa | 4260 |
| aaagctaact tgtttattgc agcttataat ggttacaaat aaagcaatag catcacaaat | 4320 |
| ttcacaaata aagcattttt ttcactgcat tctagttgtg gtttgtccaa actcatcaat | 4380 |
| gtatcttatc atgtctgtcc gcttcctcgc tcactgactc gctgcgctcg gtcgttcggc | 4440 |
| tgcggcgagc ggtatcagct cactcaaagg cggtaatacg gttatccaca gaatcagggg | 4500 |
| ataacgcagg aaagaacatg tgagcaaaag gccagcaaaa ggccaggaac cgtaaaaagg | 4560 |
| ccgcgttgct ggcgtttttc cataggctcc gcccccctga cgagcatcac aaaaatcgac | 4620 |
| gctcaagtca gaggtggcga aacccgacag gactataaag ataccaggcg tttccccctg | 4680 |
| gaagctccct cgtgcgctct cctgttccga ccctgccgct taccggatac ctgtccgcct | 4740 |
| ttctcccttc gggaagcgtg gcgctttctc atagctcacg ctgtaggtat ctcagttcgg | 4800 |
| tgtaggtcgt tcgctccaag ctgggctgtg tgcacgaacc ccccgttcag cccgaccgct | 4860 |
| gcgccttatc cggtaactat cgtcttgagt ccaacccggt aagacacgac ttatcgccac | 4920 |
| tggcagcagc cactggtaac aggattagca gagcgaggta tgtaggcggt gctacagagt | 4980 |
| tcttgaagtg gtggcctaac tacggctaca ctagaagaac agtatttggt atctgcgctc | 5040 |
| tgctgaagcc agttaccttc ggaaaaagag ttggtagctc ttgatccggc aaacaaacca | 5100 |
| ccgctggtag cggtggtttt tttgtttgca agcagcagat tacgcgcaga aaaaaaggat | 5160 |
| ctcaagaaga tcctttgatc ttttctacgg ggtctgacgc tcagtggaac gaaaactcac | 5220 |
| gttaagggat tttggtcatg agattatcaa aaaggatctt cacctagatc cttttaaatt | 5280 |
| aaaaatgaag ttttaaatca atctaaagta tatatgagta aacttggtct gacagttaga | 5340 |
| aaaactcatc gagcatcaaa tgaaactgca atttattcat atcaggatta tcaataccat | 5400 |
| atttttgaaa aagccgtttc tgtaatgaag gagaaaactc accgaggcag ttccatagga | 5460 |
| tggcaagatc ctggtatcgg tctgcgattc cgactcgtcc aacatcaata caacctatta | 5520 |
| atttcccctc gtcaaaaata aggttatcaa gtgagaaatc accatgagtg acgactgaat | 5580 |
| ccggtgagaa tggcaacagc ttatgcattt ctttccagac ttgttcaaca ggccagccat | 5640 |
| tacgctcgtc atcaaaatca ctcgcatcaa ccaaaccgtt attcattcgt gattgcgcct | 5700 |
| gagcgagacg aaatacgcga tcgctgttaa aaggacaatt acaaacagga atcgaatgca | 5760 |
| accggcgcag gaacactgcc agcgcatcaa caatattttc acctgaatca ggatattctt | 5820 |
| ctaatacctg gaatgctgtt tttccgggga tcgcagtggt gagtaaccat gcatcatcag | 5880 |
| gagtacggat aaaatgcttg atggtcggaa gaggcataaa ttccgtcagc cagtttagtc | 5940 |
| tgaccatctc atctgtaaca tcattggcaa cgctaccttt gccatgtttc agaaacaact | 6000 |
| ctggcgcatc gggcttccca tacaatcgat agattgtcgc acctgattgc ccgacattat | 6060 |
| cgcgagccca tttataccca tataaatcag catccatgtt ggaatttaat cgcggcctag | 6120 |
| agcaagacgt ttcccgttga atatggctca taacacccct tgtattactg tttatgtaag | 6180 |
| cagacagttt tattgttcat gatgatatat ttttatcttg tgcaatgtaa catcagagat | 6240 |
| tttgagacac aacaattggt cgac | 6264 |

SEQ ID NO: 20 pDNA3b plasmid pGM303 (FIG. 2H)

| | |
|---|---|
| attgattatt gactagttat taatagtaat caattacggg gtcattagtt catagcccat | 60 |
| atatggagtt ccgcgttaca taacttacgg taaatggccc gcctggctga ccgcccaacg | 120 |
| acccccgccc attgacgtca ataatgacgt atgttcccat agtaacgcca atagggactt | 180 |
| tccattgacg tcaatgggtg gagtatttac ggtaaactgc ccacttggca gtacatcaag | 240 |
| tgtatcatat gccaagtacg ccccctattg acgtcaatga cggtaaatgg cccgcctggc | 300 |
| attatgccca gtacatgacc ttatgggact ttcctacttg gcagtacatc tacgtattag | 360 |
| tcatcgctat taccatggtc gaggtgagcc ccacgttctg cttcactctc cccatctccc | 420 |
| ccccctcccc acccccaatt ttgtatttat ttatttttta attattttgt gcagcgatgg | 480 |
| gggcgggggg gggggggggg cgcgcgccag gcggggcggg gcggggcgag gggcggggcg | 540 |
| gggcgaggcg gagaggtgcg gcggcagcca atcagagcgg cgcgctccga aagtttcctt | 600 |
| ttatggcgag gcggcggcgg cggcggccct ataaaaagcg aagcgcgcgg cgggcgggag | 660 |
| tcgctgcgcg ctgccttcgc cccgtgcccc gctccgccgc cgcctcgcgc cgcccgcccc | 720 |
| ggctctgact gaccgcgtta ctcccacagg tgagcgggcg ggacggccct tctcctccgg | 780 |
| gctgtaatta gcgcttggtt taatgacggc ttgtttcttt tctgtggctg cgtgaaagcc | 840 |
| ttgaggggct ccgggagggc cctttgtgcg gggggagcgg ctcggggggt gcgtgcgtgt | 900 |
| gtgtgtgcgt ggggagcgcc gcgtgcggct ccgcgctgcc cggcggctgt gagcgctgcg | 960 |
| ggcgcggcgc ggggctttgt gcgctccgca gtgtgcgcga ggggagcgcg gccgggggcg | 1020 |
| gtgccccgcg gtgcgggggg ggctgcgagg ggaacaaagg ctgcgtgcgg ggtgtgtgcg | 1080 |
| tgggggggtg agcagggggt gtgggcgcgt cggtcgggct gcaacccccc ctgcaccccc | 1140 |
| ctccccgagt tgctgagcac ggcccggctt cgggtgcggg gctccgtacg gggcgtggcg | 1200 |
| cggggctcgc cgtgccgggc ggggggtggc ggcaggtggg ggtgccgggc ggggcggggc | 1260 |
| cgcctcgggc cggggagggc tcgggggagg ggcgcggcgg cccccggagc gccggcggct | 1320 |
| gtcgaggcgc ggcgagccgc agccattgcc ttttatggta atcgtgcgag agggcgcagg | 1380 |
| gacttccttt gtcccaaatc tgtgcggagc cgaaatctgg gaggcgccgc cgcaccccct | 1440 |
| ctagcgggcg cggggcgaag cggtgcggcg ccggcaggaa ggaaatgggc ggggagggcc | 1500 |
| ttcgtgcgtc gccgcgccgc cgtccccttc tccctctcca gcctcggggc tgtccgcggg | 1560 |
| gggacggggc agggcggggt tcggcttctg gcgtgtgacc ggcggctcta gagcctctgc | 1620 |
| taaccatgtt catgccttct tctttttcct acagctcctg ggcaacgtgc tggttattgt | 1680 |
| gctgtctcat cattttggca aagaattcct cgagcatgtg gtctgagtta aaaatcagga | 1740 |
| gcaacgacgg aggtgaagga ccagaggacg ccaacgaccc ccggggaaag ggggtgcaac | 1800 |
| acatccatat ccagccatct ctacctgttt atggacagag ggttagggat ggtgataggg | 1860 |
| gcaaacgtga ctcgtactgg tctacttctc ctagtggtag caccacaaaa ccagcatcag | 1920 |
| gttgggagag gtcaagtaaa gccgacacat ggttgctgat tctctcattc acccagtggg | 1980 |
| ctttgtcaat tgccacagtg atcatctgta tcataatttc tgctagacaa gggtatagta | 2040 |
| tgaaagagta ctcaatgact gtagaggcat tgaacatgag cagcagggag gtgaaagagt | 2100 |
| cacttaccag tctaataagg caagaggtta tagcaagggc tgtcaacatt cagagctctg | 2160 |
| tgcaaaccgg aatcccagtc ttgttgaaca aaaacagcag ggatgtcatc cagatgattg | 2220 |
| ataagtcgtg cagcagacaa gagctcactc agcactgtga gagtacgatc gcagtccacc | 2280 |
| atgccgatgg aattgcccca cttgagccac atagtttctg gagatgccct gtcggagaac | 2340 |
| cgtatcttag ctcagatcct gaaatctcat tgctgcctgg tccgagcttg ttatctggtt | 2400 |
| ctacaacgat ctctggatgt gttaggctcc cttcactctc aattggcgag gcaatctatg | 2460 |
| cctattcatc aaatctcatt acacaaggtt gtgctgacat agggaaatca tatcaggtcc | 2520 |
| tgcagctagg gtacatatca ctcaattcag atatgttccc tgatcttaac cccgtagtgt | 2580 |
| cccacactta tgacatcaac gacaatcgga aatcatgctc tgtggtggca accgggacta | 2640 |
| ggggttatca gctttgctcc atgccgactg tagacgaaag aaccgactac tctagtgatg | 2700 |
| gtattgagga tctggtcctt gatgtcctgg atctcaaagg gagaactaag tctcaccggt | 2760 |
| atcgcaacag cgaggtagat cttgatcacc cgttctctgc actatacccc agtgtaggca | 2820 |
| acggcattgc aacagaaggc tcattgatat ttcttgggta tggtggacta accacccctc | 2880 |
| tgcagggtga tacaaaatgt aggacccaag gatgccaaca ggtgtcgcaa gacacatgca | 2940 |
| atgaggctct gaaaattaca tggctaggag ggaaacaggt ggtcagcgtg atcatccagg | 3000 |
| tcaatgacta tctctcagag aggccaaaga taagagtcac aaccattcca atcactcaaa | 3060 |
| actatctcgg ggcggaaggt agattattaa aattgggtga tcgggtgtac atctatacaa | 3120 |
| gatcatcagg ctggcactct caactgcaga taggagtact tgatgtcagc caccctttga | 3180 |
| ctatcaactg gacacctcat gaagccttgt ctagaccagg aaataaagag tgcaattggt | 3240 |
| acaataagtg tccgaaggaa tgcatatcag gcgtatacac tgatgcttat ccattgtccc | 3300 |
| ctgatgcagc taacgtcgct accgtcacgc tatatgccaa tacatcgcgt gtcaacccaa | 3360 |
| caatcatgta ttctaacact actaacatta taaatatgtt aaggataaag gatgttcaat | 3420 |
| tagaggctgc atataccacg acatcgtgta tcacgcattt tggtaaaggc tactgctttc | 3480 |
| acatcatcga gatcaatcag aagagcctga ataccttaca gccgatgctc tttaagacta | 3540 |
| gcatccctaa attatgcaag gccgagtctt aagcggccgc gcatgcgaat tcactcctca | 3600 |
| ggtgcaggct gcctatcaga aggtggtggc tggtgtggcc aatgccctgg ctcacaaata | 3660 |
| ccactgagat ctttttccct ctgccaaaaa ttatggggac atcatgaagc cccttgagca | 3720 |
| tctgacttct ggctaataaa ggaaatttat tttcattgca atagtgtgtt ggaatttttt | 3780 |
| gtgtctctca ctcggaagga catatgggag ggcaaatcat ttaaaacatc agaatgagta | 3840 |
| tttggtttag agtttggcaa catatgccca tatgctggct gccatgaaca aaggttggct | 3900 |
| ataaagaggt catcagtata tgaaacagcc ccctgctgtc tattccttat tccatagaaa | 3960 |
| agccttgact tgaggttaga ttttttttat attttgtttt gtgttatttt tttctttaac | 4020 |
| atccctaaaa ttttccttac atgttttact agccagattt ttcctcctct cctgactact | 4080 |
| cccagtcata gctgtccctc ttctcttatg gagatccctc gacctgcagc ccaagcttgg | 4140 |
| cgtaatcatg gtcatagctg tttcctgtgt gaaattgtta tccgctcaca attccacaca | 4200 |
| acatacgagc cggaagcata aagtgtaaag cctggggtgc ctaatgagtg agctaactca | 4260 |
| cattaattgc gttgcgctca ctgcccgctt tccagtcggg aaacctgtcg tgccagcgga | 4320 |
| tccgcatctc aattagtcag caaccatagt cccgccccta actccgccca tcccgcccct | 4380 |
| aactccgccc agttccgccc attctccgcc ccatggctga ctaatttttt ttatttatgc | 4440 |
| agaggccgag gccgcctcgg cctctgagct attccagaag tagtgaggag gcttttttgg | 4500 |
| aggcctaggc ttttgcaaaa agctaacttg tttattgcag cttataatgg ttacaaataa | 4560 |
| agcaatagca tcacaaattt cacaaataaa gcattttttt cactgcattc tagttgtggt | 4620 |
| ttgtccaaac tcatcaatgt atcttatcat gtctgtccgc ttcctcgctc actgactcgc | 4680 |
| tgcgctcggt cgttcggctg cggcgagcgg tatcagctca ctcaaaggcg gtaatacggt | 4740 |
| tatccacaga atcaggggat aacgcaggaa agaacatgtg agcaaaaggc cagcaaaagg | 4800 |
| ccaggaaccg taaaaaggcc gcgttgctgg cgtttttcca taggctccgc ccccctgacg | 4860 |
| agcatcacaa aaatcgacgc tcaagtcaga ggtggcgaaa cccgacagga ctataaagat | 4920 |
| accaggcgtt tccccctgga agctccctcg tgcgctctcc tgttccgacc ctgccgctta | 4980 |
| ccggatacct gtccgccttt ctcccttcgg gaagcgtggc gctttctcat agctcacgct | 5040 |
| gtaggtatct cagttcggtg taggtcgttc gctccaagct gggctgtgtg cacgaacccc | 5100 |
| ccgttcagcc cgaccgctgc gccttatccg gtaactatcg tcttgagtcc aacccggtaa | 5160 |
| gacacgactt atcgccactg gcagcagcca ctggtaacag gattagcaga gcgaggtatg | 5220 |
| taggcggtgc tacagagttc ttgaagtggt ggcctaacta cggctacact agaagaacag | 5280 |
| tatttggtat ctgcgctctg ctgaagccag ttaccttcgg aaaaagagtt ggtagctctt | 5340 |
| gatccggcaa acaaaccacc gctggtagcg gtggtttttt tgtttgcaag cagcagatta | 5400 |
| cgcgcagaaa aaaaggatct caagaagatc ctttgatctt ttctacgggg tctgacgctc | 5460 |
| agtggaacga aaactcacgt taagggattt tggtcatgag attatcaaaa aggatcttca | 5520 |
| cctagatcct tttaaattaa aaatgaagtt ttaaatcaat ctaaagtata tatgagtaaa | 5580 |
| cttggtctga cagttagaaa aactcatcga gcatcaaatg aaactgcaat ttattcatat | 5640 |
| caggattatc aataccatat ttttgaaaaa gccgtttctg taatgaagga gaaaactcac | 5700 |
| cgaggcagtt ccataggatg gcaagatcct ggtatcggtc tgcgattccg actcgtccaa | 5760 |
| catcaataca acctattaat ttcccctcgt caaaaataag gttatcaagt gagaaatcac | 5820 |
| catgagtgac gactgaatcc ggtgagaatg gcaacagctt atgcatttct ttccagactt | 5880 |
| gttcaacagg ccagccatta cgctcgtcat caaaatcact cgcatcaacc aaaccgttat | 5940 |
| tcattcgtga ttgcgcctga gcgagacgaa atacgcgatc gctgttaaaa ggacaattac | 6000 |
| aaacaggaat cgaatgcaac cggcgcagga acactgccag cgcatcaaca atattttcac | 6060 |
| ctgaatcagg atattcttct aatacctgga atgctgtttt tccggggatc gcagtggtga | 6120 |
| gtaaccatgc atcatcagga gtacggataa aatgcttgat ggtcggaaga ggcataaatt | 6180 |
| ccgtcagcca gtttagtctg accatctcat ctgtaacatc attggcaacg ctacctttgc | 6240 |
| catgtttcag aaacaactct ggcgcatcgg gcttcccata caatcgatag attgtcgcac | 6300 |
| ctgattgccc gacattatcg cgagcccatt tatacccata taaatcagca tccatgttgg | 6360 |
| aatttaatcg cggcctagag caagacgttt cccgttgaat atggctcata acaccccttg | 6420 |
| tattactgtt tatgtaagca gacagtttta ttgttcatga tgatatattt ttatcttgtg | 6480 |
| caatgtaaca tcagagattt tgagacacaa caattggtcg ac | 6522 |

SEQ ID NO: 21 pDNA1* plasmid pSIV-2V-Transactivator (FIG. 2C)
GGTACCTCAATATTGGCCATTAGCCATATTATTCATTGGTTATATAGCATAAATCAATATTGGCTATTGGCCATT
GCATACGTTGTATCTATATCATAATATGTACATTTATATTGGCTCATGTCCAATATGACCGCCATGTTGGCATTG
ATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGCCCACTTGGC
AGTACATCAAGTGTATCATATGCCAAGTCCGCCCCCTATTGACGTCAATGACGGTAAATGGCCCGCCTGGCATTA
TGCCCAGTACATGACCTTACGGGACTTTCCTACTTGGCAGTACATCTACGTATTAGTCATCGCTATTACCATGGT
GATGCGGTTTTGGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTGCGATCGCCC
GCCCCGTTGACGCAAATGGGCGGTAGGCGTGTACGGTGGGAGGTCTATATAAGCAGAGCTCGCTGGCTTGTAACT
CAGTCTCTTACTAGGAGACCAGCTTGAGCCTGGGTGTTCGCTGGTTAGCCTAACCTGGTTGGCCACCAGGGGTAA
GGACTCCTTGGCTTAGAAAGCTAATAAACTTGCCTGCATTAGAGCTTATCTGAGTCAAGTGTCCTCATTGACGCC
TCACTCTCTTGAACGGGAATCTTCCTTACTGGGTTCTCTCTCTGACCCAGGCGAGAGAAACTCCAGCAGTGGCGC
CCGAACAGGGACTTGAGTGAGAGTGTAGGCACGTACAGCTGAGAAGGCGTCGGACGCGAAGGAAGCGCGGGGTGC
GACGCGACCAAGAAGGAGACTTGGTGAGTAGGCTTCTCGAGTGCCGGGAAAAAGCTCGAGCCTAGTTAGAGGACT
AGGAGAGGCCGTAGCCGTAACTACTCTTGGGCAAGTAGGGCAGGCGGTGGGTACGCAATGGGGGCGGCTACCTCA
GCACTAAATAGGAGACAATTAGACCAATTTGAGAAAATACGACTTCGCCCGAACGGAAAGAAAAAGTACCAAATT
AAACATTTAATATGGGCAGGCAAGGAGATGGAGCGCTTCGGCCTCCATGAGAGGTTGTTGGAGACAGAGGAGGGG
TGTAAAAGAATCATAGAAGTCCTCTACCCCCTAGAACCAACAGGATCGGAGGGCTTAAAAAGTCTGTTCAATCTT
GTGTGCGTGCTATATTGCTTGCACAAGGAACAGAAAGTGAAAGACACAGAGGAAGCAGTAGCAACAGTAAGACAA
CACTGCCATCTAGTGGAAAAAGAAAAAAGTGCAACAGAGACATCTAGTGGACAAAAGAAAAATGACAAGGGAATA
GCAGCGCCACCTGGTGGCAGTCAGAATTTTCCAGCGCAACAACAAGGAAATGCCTGGGTACATGTACCCTTGTCA
CCGCGCACCTTAAATGCGTGGGTAAAAGCAGTAGAGGAGAAAAAATTTGGAGCAGAAATAGTACCCATTTTTTTG
TTTCAAGCCCTATCGAATTCCCGTTTGTGCTAGGGTTCTTAGGCTTCTTGGGGGCTGCTGGAACTGCAATGGGAG
CAGCGGCGACAGCCCTGACGGTCCAGTCTCAGCATTTGCTTGCTGGGATACTGCAGCAGCAGAAGAATCTGCTGG
CGGCTGTGGAGGCTCAACAGCAGATGTTGAAGCTGACCATTTGGGGTGTTAAAAACCTCAATGCCCGCGTCACAG
CCCTTGAGAAGTACCTAGAGGATCAGGCACGACTAAACTCCTGGGGGTGCGCATGGAAACAAGTATGTCATACCA
CAGTGGAGTGGCCCTGGACAAATCGGACTCCGGATTGGCAAAATATGACTTGGTTGGAGTGGGAAAGACAAATAG
CTGATTTGGAAAGCAACATTACGAGACAATTAGTGAAGGCTAGAGAACAAGAGGAAAAGAATCTAGATGCCTATC
AGAAGTTAACTAGTTGGTCAGATTTCTGGTCTTGGTTCGATTTCTCAAAATGGCTTAACATTTTAAAAATGGGAT
TTTTAGTAATAGTAGGAATAATAGGGTTAAGATTACTTTACACAGTATATGGATGTATAGTGAGGGTTAGGCAGG
GATATGTTCCTCTATCTCCACAGATCCATATCCGCGGCAATTTTAAAAGAAAGGGAGGAATAGGGGGACAGACTT
CAGCAGAGAGACTAATTAATATAATAACAACACAATTAGAAATACAACATTTACAAACCAAAATTCAAAAAATTT
TAAATTTTAGAGCCGCGGACGAGCTTGCATGCCTGCAGGTCGAAGCGGAGTACTGTCCTCCGAGTTTAAAAGCGG
AGTACTGTCCTCCGAGGATATCAGCGGAGTACTGTCCTCCGAGTCGCGAAGCGGAGTACTGTCCTCCGAGATCGA
TGTCGACCCCGCCCAGCGTCTTGTCATTGGCGAATTCGAACACGCAGATGCAGTCGGGGCGGCGCGGTCCGAGGT
CCACTTCGCATATTAAGGTGACGCGTGTGGCCTCGAATCGCCTGGAGACGCCATCCACGCTGTTTTGACCTCCAT
AGAAGACACCGGGACCGATCCAGCCTCCGCGGCCGGGAACGGTGCATTGGAACGCGGATTCCCCGTGTTAATTAA
CAGGTAAGTGTCTTCCTCCTGTTTCCTTCCCCTGCTATTCTGCTCAACCTTCCTATCAGAAACTGCAGTATCTGT
ATTTTTGCTAGCAGTAATACTAACGGTTCTTTTTTTCTCTTCACAGGCCACCAAGCTACCGGTCCACCATGGACT
CCCAGCAGCCAGATCTGAAGCTACTGTCTTCTATCGAACAAGCATGCGATATTTGCCGACTTAAAAAGCTCAAGT
GCTCCAAAGAAAAACCGAAGTGCGCCAAGTGTCTGAAGAACAACTGGGAGTGTCGCTACTCTCCCAAAACCAAAA
GGTCTCCGCTGACTAGGGCACATCTGACAGAAGTGGAATCAAGGCTAGAAAGACTGGAACAGCTATTTCTACTGA
TTTTTCCTCGAGAAGACCTTGACATGATTTTGAAAATGGATTCTTTACAGGATATAAAAGCATTGTTAGAATTCC
CGGGTGTCGACCAGAAAAAGTTCAATAAAGTCAGAGTTGTGAGAGCACTGGATGCTGTTGCTCTCCCACAGCCAG
TGGGCGTTCCAAATGAAAGCCAAGCCCTAAGCCAGAGATTCACTTTTTCACCAGGTCAAGACATACAGTTGATTC
CACCACTGATCAACCTGTTAATGAGCATTGAACCAGATGTGATCTATGCAGGACATGACAACACAAAACCTGACA
CCTCCAGTTCTTTGCTGACAAGTCTTAATCAACTAGGCGAGAGGCAACTTCTTTCAGTAGTCAAGTGGTCTAAAT
CATTGCCAGGTTTTCGAAACTTACATATTGATGACCAGATAACTCTCATTCAGTATTCTTGGATGAGCTTAATGG
TGTTTGGTCTAGGATGGAGATCCTACAAACACGTCAGTGGGCAGATGCTGTATTTTGCACCTGATCTAATACTAA
ATGAACAGCGGATGAAAGAATCATCATTCTATTCATTATGCCTTACCATGTGGCAGATCCCACAGGAGTTTGTCA
AGCTTCAAGTTAGCCAAGAAGAGTTCCTCTGTATGAAAGTATTGTTACTTCTTAATACAATTCCTTTGGAAGGGC
TACGAAGTCAAACCCAGTTTGAGGAGATGAGGTCAAGCTACATTAGAGAGCTCATCAAGGCAATTGGTTTGAGGC
AAAAAGGAGTTGTGTCGAGCTCACAGCGTTTCTATCAACTTACAAAACTTCTTGATAACTTGCATGATCTTGTCA
AACAACTTCATCTGTACTGCTTGAATACATTTATCCAGTCCCGGGCACTGAGTGTTGAATTTCCAGAAATGATGT
CTGAAGTTATTGCTGGGTCGACGCCCATGGAATTCCAGTACCTGCCAGATACAGACGATCGTCACCGGATTGAGG
AGAAACGTAAAAGGACATATGAGACCTTCAAGAGCATCATGAAGAAGAGTCCTTTCAGCGGACCCACCGACCCCC
GGCCTCCACCTCGACGCATTGCTGTGCCTTCCCGCAGCTCAGCTTCTGTCCCCAAGCCAGCACCCCAGCCCTATC
CCTTTACGTCATCCCTGAGCACCATCAACTATGATGAGTTTCCCACCATGGTGTTTCCTTCTGGGCAGATCAGCC
AGGCCTCGGCCTTGGCCCCGGCCCCTCCCCAAGTCCTGCCCCAGGCTCCAGCCCCTGCCCCTGCTCCAGCCATGG
TATCAGCTCTGGCCCAGGCCCCAGCCCCTGTCCCAGTCCTAGCCCCAGGCCCTCCTCAGGCTGTGGCCCCACCTG
CCCCCAAGCCCACCCAGGCTGGGGAAGGAACGCTGTCAGAGGCCCTGCTGCAGCTGCAGTTTGATGATGAAGACC
TGGGGGCCTTGCTTGGCAACAGCACAGACCCAGCTGTGTTCACAGACCTGGCATCCGTCGACAACTCCGAGTTTC
AGCAGCTGCTGAACCAGGGCATACCTGTGGCCCCCCACACAACTGAGCCCATGCTGATGGAGTACCCTGAGGCTA
TAACTCGCCTAGTGACAGGGGCCCAGAGGCCCCCCGACCCAGCTCCTGCTCCACTGGGGGCCCCGGGGCTCCCCA
ATGGCCTCCTTTCAGGAGATGAAGACTTCTCCTCCATTGCGGACATGGACTTCTCAGCCCTGCTGAGTCAGATCA
GCTCCTAAGGATCCTCCGGACTAGAAAAGCCGAATTCTGCAGGAATTGGGTGGCATCCCTGTGACCCCTCCCCAG
TGCCTCTCCTGGCCCTGGAAGTTGCCACTCCAGTGCCCACCAGCCTTGTCCTAATAAAATTAAGTTGCATCATTT
TGTCTGACTAGGTGTCCTTCTATAATATTATGGGGTGGAGGGGGGTGGTATGGAGCAAGGGGCAAGTTGGGAAGA
CAACCTGTAGGGCTCGAGGGGGGGCCCGAAACCCGCTGATCAGCCTCGAGGCCCAATCAACCTCTGGATTACAAA
ATTTGTGAAAGATTGACTGGTATTCTTAACTATGTTGCTCCTTTTACGCTATGTGGATACGCTGCTTTAATGCCT
TTGTATCATGCTATTGCTTCCCGTATGGCTTTCATTTTCTCCTCCTTGTATAAATCCTGGTTGCTGTCTCTTTAT
GAGGAGTTGTGGCCCGTTGTCAGGCAACGTGGCGTGGTGTGCACTGTGTTTGCTGACGCAACCCCCACTGGTTGG
GGCATTGCCACCACCTGTCAGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATTCCGTGGTGTTGTCGGGGAAA
TCATCGTCCTTTCCTTGGCTGCTCGCCTGTGTTGCCACCTGGATTCTGCGCGGGACGTCCTTCTGCTACGTCCCT
TCGGCCCTCAATCCAGCGGACCTTCCTTCCCGCGGCCTGCTGCCGGCTCTGCGGCCTCTTCCGCGTCTTCGCCTT
CGCCCTCAGACGAGTCGGATCTCCCTTTGGGCCGCCTCCCCGCAAGCTTCGCACTTTTTAAAAGAAAAGGGAGGA
CTGGATGGGATTTATTACTCCGATAGGACGCTGGCTTGTAACTCAGTCTCTTACTAGGAGACCAGCTTGAGCCTG
GGTGTTCGCTGGTTAGCCTAACCTGGTTGGCCACCAGGGGTAAGGACTCCTTGGCTTAGAAAGCTAATAAACTTG
CCTGCATTAGAGCTCTTACGCGTCCCGGGCTCGAGATCCGCATCTCAATTAGTCAGCAACCATAGTCCCGCCCCT
AACTCCGCCCATCCCGCCCCTAACTCCGCCCAGTTCCGCCCATTCTCCGCCCCATGGCTGACTAATTTTTTTTAT
TTATGCAGAGGCCGAGGCCGCCTCGGCCTCTGAGCTATTCCAGAAGTAGTGAGGAGGCTTTTTTGGAGGCCTAGG
CTTTTGCAAAAAGCTAACTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTATCATGTCTGTCCG
CTTCCTCGCTCACTGACTCGCTGCGCTCGGTCGTTCGGCTGCGGCGAGCGGTATCAGCTCACTCAAAGGCGGTAA
TACGGTTATCCACAGAATCAGGGGATAACGCAGGAAAGAACATGTGAGCAAAAGGCCAGCAAAAGGCCAGGAACC
GTAAAAAGGCCGCGTTGCTGGCGTTTTTCCATAGGCTCCGCCCCCCTGACGAGCATCACAAAAATCGACGCTCAA
GTCAGAGGTGGCGAAACCCGACAGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGCGTGGCGCTTTCTCATAGCT
CACGCTGTAGGTATCTCAGTTCGGTGTAGGTCGTTCGCTCCAAGCTGGGCTGTGTGCACGAACCCCCCGTTCAGC
CCGACCGCTGCGCCTTATCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCTTGAAGTGGTGGCCTAACT
ACGGCTACACTAGAAGAACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCAGCAGATTACGCGCAGAA
AAAAAGGATCTCAAGAAGATCCTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATTAAAAATGAAGTTTTAAATCAA
TCTAAAGTATATATGAGTAAACTTGGTCTGACAGTTAGAAAAACTCATCGAGCATCAAATGAAACTGCAATTTAT
TCATATCAGGATTATCAATACCATATTTTTGAAAAAGCCGTTTCTGTAATGAAGGAGAAAACTCACCGAGGCAGT
TCCATAGGATGGCAAGATCCTGGTATCGGTCTGCGATTCCGACTCGTCCAACATCAATACAACCTATTAATTTCC
CCTCGTCAAAAATAAGGTTATCAAGTGAGAAATCACCATGAGTGACGACTGAATCCGGTGAGAATGGCAACAGCT
TATGCATTTCTTTCCAGACTTGTTCAACAGGCCAGCCATTACGCTCGTCATCAAAATCACTCGCATCAACCAAAC
CGTTATTCATTCGTGATTGCGCCTGAGCGAGACGAAATACGCGATCGCTGTTAAAAGGACAATTACAAACAGGAA
TCGAATGCAACCGGCGCAGGAACACTGCCAGCGCATCAACAATATTTTCACCTGAATCAGGATATTCTTCTAATA
CCTGGAATGCTGTTTTTCCGGGGATCGCAGTGGTGAGTAACCATGCATCATCAGGAGTACGGATAAAATGCTTGA
TGGTCGGAAGAGGCATAAATTCCGTCAGCCAGTTTAGTCTGACCATCTCATCTGTAACATCATTGGCAACGCTAC
CTTTGCCATGTTTCAGAAACAACTCTGGCGCATCGGGCTTCCCATACAATCGATAGATTGTCGCACCTGATTGCC
CGACATTATCGCGAGCCCATTTATACCCATATAAATCAGCATCCATGTTGGAATTTAATCGCGGCCTAGAGCAAG
ACGTTTCCCGTTGAATATGGCTCATAACACCCCTTGTATTACTGTTTATGTAAGCAGACAGTTTTATTGTTCATG
ATGATATATTTTTATCTTGTGCAATGTAACATCAGAGATTTTGAGACACAACAATTGGTCGACGGATCC
SEQ ID NO: 22 pDNA1^{ta+} plasmid pSIV-1V-GMCSF (FIG. 2A)
GGTACCTCAATATTGGCCATTAGCCATATTATTCATTGGTTATATAGCATAAATCAATATTGGCTATTGGCCATT
GCATACGTTGTATCTATATCATAATATGTACATTTATATTGGCTCATGTCCAATATGACCGCCATGTTGGCATTG
ATTATTGACTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATGACGTATGT
TCCCATAGTAACGCCAATAGGGACTTTCCATTGACGTCAATGGGTGGAGTATTTACGGTAAACTGCCCACTTGGC
AGTACATCAAGTGTATCATATGCCAAGTCCGCCCCCTATTGACGTCAATGACGGTAAATGGCCCGCCTGGCATTA
TGCCCAGTACATGACCTTACGGGACTTTCCTACTTGGCAGTACATCTACGTATTAGTCATCGCTATTACCATGGT
GATGCGGTTTTGGCAGTACACCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTGCGATCGCCC
GCCCCGTTGACGCAAATGGGCGGTAGGCGTGTACGGTGGGAGGTCTATATAAGCAGAGCTCGCTGGCTTGTAACT
CAGTCTCTTACTAGGAGACCAGCTTGAGCCTGGGTGTTCGCTGGTTAGCCTAACCTGGTTGGCCACCAGGGGTAA
GGACTCCTTGGCTTAGAAAGCTAATAAACTTGCCTGCATTAGAGCTTATCTGAGTCAAGTGTCCTCATTGACGCC
TCACTCTCTTGAACGGGAATCTTCCTTACTGGGTTCTCTCTCTGACCCAGGCGAGAGAAACTCCAGCAGTGGCGC
CCGAACAGGGACTTGAGTGAGAGTGTAGGCACGTACAGCTGAGAAGGCGTCGGACGCGAAGGAAGCGCGGGGTGC
GACGCGACCAAGAAGGAGACTTGGTGAGTAGGCTTCTCGAGTGCCGGGAAAAAGCTCGAGCCTAGTTAGAGGACT
AGGAGAGGCCGTAGCCGTAACTACTCTTGGGCAAGTAGGGCAGGCGGTGGGTACGCAATGGGGGGGGCTACCTCA
GCACTAAATAGGAGACAATTAGACCAATTTGAGAAAATACGACTTCGCCCGAACGGAAAGAAAAAGTACCAAATT
AAACATTTAATATGGGCAGGCAAGGAGATGGAGCGCTTCGGCCTCCATGAGAGGTTGTTGGAGACAGAGGAGGGG
TGTAAAAGAATCATAGAAGTCCTCTACCCCCTAGAACCAACAGGATCGGAGGGCTTAAAAAGTCTGTTCAATCTT
GTGTGCGTGCTATATTGCTTGCACAAGGAACAGAAAGTGAAAGACACAGAGGAAGCAGTAGCAACAGTAAGACAA
CACTGCCATCTAGTGGAAAAAGAAAAAAGTGCAACAGAGACATCTAGTGGACAAAAGAAAAATGACAAGGGAATA
GCAGCGCCACCTGGTGGCAGTCAGAATTTTCCAGCGCAACAACAAGGAAATGCCTGGGTACATGTACCCTTGTCA
CCGCGCACCTTAAATGCGTGGGTAAAAGCAGTAGAGGAGAAAAAATTTGGAGCAGAAATAGTACCCATTTTTTTG
TTTCAAGCCCTATCGAATTCCCGTTTGTGCTAGGGTTCTTAGGCTTCTTGGGGGCTGCTGGAACTGCAATGGGAG
CAGCGGCGACAGCCCTGACGGTCCAGTCTCAGCATTTGCTTGCTGGGATACTGCAGCAGCAGAAGAATCTGCTGG
CGGCTGTGGAGGCTCAACAGCAGATGTTGAAGCTGACCATTTGGGGTGTTAAAAACCTCAATGCCCGCGTCACAG
CCCTTGAGAAGTACCTAGAGGATCAGGCACGACTAAACTCCTGGGGGTGCGCATGGAAACAAGTATGTCATACCA
CAGTGGAGTGGCCCTGGACAAATCGGACTCCGGATTGGCAAAATATGACTTGGTTGGAGTGGGAAAGACAAATAG
CTGATTTGGAAAGCAACATTACGAGACAATTAGTGAAGGCTAGAGAACAAGAGGAAAAGAATCTAGATGCCTATC
AGAAGTTAACTAGTTGGTCAGATTTCTGGTCTTGGTTCGATTTCTCAAAATGGCTTAACATTTTAAAAATGGGAT
TTTTAGTAATAGTAGGAATAATAGGGTTAAGATTACTTTACACAGTATATGGATGTATAGTGAGGGTTAGGCAGG
GATATGTTCCTCTATCTCCACAGATCCATATCCGCGGCAATTTTAAAAGAAAGGGAGGAATAGGGGGACAGACTT
CAGCAGAGAGACTAATTAATATAATAACAACACAATTAGAAATACAACATTTACAAACCAAAATTCAAAAAATTT
TAAATTTTAGAGCCGCGGAGACCGCCTCAGAAGGCATGCCTGCTATTGTCTTCCCAATCCTCCCCCTTGCTGTCC
TGCCCCACCCCACCCCCCAGAATAGAATGACACCTACTCAGACAATGCAATGCAATTTCCTCATTTTATTAGGAA
AGGACAGTGGGAGTGGCACCTTCCAGGGTCAAGGAAGGCACAGGGGAGGGGCAAACAACAGATGGCTGGCAACTA
GAAGGCACAGTCGAGGCTGATCGGATCCTTAGGAGCTGATCTGACTCAGCAGGGCTGAGAAGTCCATGTCCGCAA
TGGAGGAGAAGTCTTCATCTCCTGAAAGGAGGCCATTGGGGAGCCCCGGGGCCCCCAGTGGAGCAGGAGCTGGGT
CGGGGGGCCTCTGGGCCCCTGTCACTAGGCGAGTTATAGCCTCAGGGTACTCCATCAGCATGGGCTCAGTTGTGT
GGGGGGCCACAGGTATGCCCTGGTTCAGCAGCTGCTGAAACTCGGAGTTGTCGACGGATGCCAGGTCTGTGAACA
CAGCTGGGTCTGTGCTGTTGCCAAGCAAGGCCCCCAGGTCTTCATCATCAAACTGCAGCTGCAGCAGGGCCTCTG
ACAGCGTTCCTTCCCCAGCCTGGGTGGGCTTGGGGGCAGGTGGGGCCACAGCCTGAGGAGGGCCTGGGGCTAGGA
CTGGGACAGGGGCTGGGGCCTGGGCCAGAGCTGATACCATGGCTGGAGCAGGGGCAGGGGCTGGAGCCTGGGGCA
GGACTTGGGGAGGGGCCGGGGCCAAGGCCGAGGCCTGGCTGATCTGCCCAGAAGGAAACACCATGGTGGGAAACT
CATCATAGTTGATGGTGCTCAGGGATGACGTAAAGGGATAGGGCTGGGGTGCTGGCTTGGGGACAGAAGCTGAGC
TGCGGGAAGGCACAGCAATGCGTCGAGGTGGAGGCCGGGGGTCGGTGGGTCCGCTGAAAGGACTCTTCTTCATGA
TGCTCTTGAAGGTCTCATATGTCCTTTTACGTTTCTCCTCAATCCGGTGACGATCGTCTGTATCTGGCAGGTACT
GGAATTCCATGGGCGTCGACCCAGCAATAACTTCAGACATCATTTCTGGAAATTCAACACTCAGTGCCCGGGACT
GGATAAATGTATTCAAGCAGTACAGATGAAGTTGTTTGACAAGATCATGCAAGTTATCAAGAAGTTTTGTAAGTT
GATAGAAACGCTGTGAGCTCGACACAACTCCTTTTTGCCTCAAACCAATTGCCTTGATGAGCTCTCTAATGTAGC
TTGACCTCATCTCCTCAAACTGGGTTTGACTTCGTAGCCCTTCCAAAGGAATTGTATTAAGAAGTAACAATACTT
TCATACAGAGGAACTCTTCTTGGCTAACTTGAAGCTTGACAAACTCCTGTGGGATCTGCCACATGGTAAGGCATA
ATGAATAGAATGATGATTCTTTCATCCGCTGTTCATTTAGTATTAGATCAGGTGCAAAATACAGCATCTGCCCAC
TGACGTGTTTGTAGGATCTCCATCCTAGACCAAACACCATTAAGCTCATCCAAGAATACTGAATGAGAGTTATCT
GGTCATCAATATGTAAGTTTCGAAAACCTGGCAATGATTTAGACCACTTGACTACTGAAAGAAGTTGCCTCTCGC
CTAGTTGATTAAGACTTGTCAGCAAAGAACTGGAGGTGTCAGGTTTTGTGTTGTCATGTCCTGCATAGATCACAT
CTGGTTCAATGCTCATTAACAGGTTGATCAGTGGTGGAATCAACTGTATGTCTTGACCTGGTGAAAAAGTGAATC
TCTGGCTTAGGGCTTGGCTTTCATTTGGAACGCCCACTGGCTGTGGGAGAGCAACAGCATCCAGTGCTCTCACAA
CTCTGACTTTATTGAACTTTTTCTGGTCGACACCCGGGAATTCTAACAATGCTTTTATATCCTGTAAAGAATCCA
TTTTCAAAATCATGTCAAGGTCTTCTCGAGGAAAAATCAGTAGAAATAGCTGTTCCAGTCTTTCTAGCCTTGATT
CCACTTCTGTCAGATGTGCCCTAGTCAGCGGAGACCTTTTGGTTTTGGGAGAGTAGCGACACTCCCAGTTGTTCT
TCAGACACTTGGCGCACTTCGGTTTTTCTTTGGAGCACTTGAGCTTTTTAAGTCGGCAAATATCGCATGCTTGTT
CGATAGAAGACAGTAGCTTCAGATCTGGCTGCTGGGAGTCCATGGTGGACCGGTAGCTTGGTGGCCTGTGAAGAG
AAAAAAAGAACCGTTAGTATTACTGCTAGCAAACTCACAGGAGGGAGAAGGCAGAAGCTTGAATGTTCACAGAGA
CTACTGCACTTATATATGGTTCTCCCCCACCCTGGGGAAAAAGGTGGAGCCAGTACACCACATCACTTTCCCAGT
TTACCCAAGCCCCACCTTCTCTAGGCACCAGTTCAATTGCCCACCCCTCCCCCCAACTTCTCAGGGACTGTGGGC
CATGTGCTCTCTGCCCACTGAGGGGCACTCAGCCCTCAAGCATGCTCTTCTCCACTAGTGAATTCCCATGGTAAT
AGCAATGACTAATACATAGATGTACTGCCAAGTAGGAAAGTCCCATAAGGTCATGTACTGGGCATAATGCCAGGC
AGGCCATTTACCATCATTGACATCAATAGGGGGCATACTTGGCATATGATACACTTGATGTACTGCCAAGTGGGC
AGTTACCATAAATACTCCACCCATTGACATCAATGGAAAGTCCCTATTGGCATTACATGGGAACATACATCATTA
TTGACATCATTGGGCAGGGGTCATTGGGCAGTCAGCCAGGCAGGCCATTTACCATAAGTTATGTAACcttctgag
gcggaaagaaccagctggggctctagggggtatccccaccgagctcttacgcgggtcgaagcggagtactgtcct
ccgagtggagtactgtcctccgagcggagtactgtcctccgagtcgagggtcgaagcggagtactgtcctccgag
tggagtactgtcctccgagcggagtactgtcctccgagtcgactctagAGGGTATATAATGaagcttctgccttc
tccctcctgtaacgttgagtttgctagccaccatgtggctgcagaacctgctgttcctgggcattgtggtgtaca
gcctgtctgcccctacaagatcccctatcacagtgaccagaccttggaaacatgtggaagccatcaaagaggccc
tgaatctgctggatgacatgcctgtgacactgaatgaagaggtggaagtggtgtccaatgagttcagcttcaaga
aactgacctgtgtgcagaccaggctgaagatttttgagcagggcctgagaggcaacttcaccaagctgaaagggg
ctctgaacatgacagccagctactaccagacctactgtcctcctacacctgagacagactgtgaaacccaagtga
ccacctatgctgacttcattgacagcctcaagaccttcctgacagacatcccctttgagtgcaagaaacctggcc
agaagtgagggcccacccagctttcttgtacaaagtggtgataatcgaattcAATCAACCTCTGGATTACAAAAT
TTGTGAAAGATTGACTGGTATTCTTAACTATGTTGCTCCTTTTACGCTATGTGGATACGCTGCTTTAATGCCTTT
GTATCATGCTATTGCTTCCCGTATGGCTTTCATTTTCTCCTCCTTGTATAAATCCTGGTTGCTGTCTCTTTATGA
GGAGTTGTGGCCCGTTGTCAGGCAACGTGGCGTGGTGTGCACTGTGTTTGCTGACGCAACCCCCACTGGTTGGGG
CATTGCCACCACCTGTCAGCTCCTTTCCGGGACTTTCGCTTTCCCCCTCCCTATTGCCACGGCGGAACTCATCGC
CGCCTGCCTTGCCCGCTGCTGGACAGGGGCTCGGCTGTTGGGCACTGACAATTCCGTGGTGTTGTCGGGGAAATC
ATCGTCCTTTCCTTGGCTGCTCGCCTGTGTTGCCACCTGGATTCTGCGCGGGACGTCCTTCTGCTACGTCCCTTC
GGCCCTCAATCCAGCGGACCTTCCTTCCCGCGGCCTGCTGCCGGCTCTGCGGCCTCTTCCGCGTCTTCGCCTTCG
CCCTCAGACGAGTCGGATCTCCCTTTGGGCCGCCTCCCCGCAAGCTTCGCACTTTTTAAAAGAAAAGGGAGGACT
GGATGGGATTTATTACTCCGATAGGACGCTGGCTTGTAACTCAGTCTCTTACTAGGAGACCAGCTTGAGCCTGGG
TGTTCGCTGGTTAGCCTAACCTGGTTGGCCACCAGGGGTAAGGACTCCTTGGCTTAGAAAGCTAATAAACTTGCC
TGCATTAGAGCTCTTACGCGTCCCGGGCTCGAGATCCGCATCTCAATTAGTCAGCAACCATAGTCCCGCCCCTAA
CTCCGCCCATCCCGCCCCTAACTCCGCCCAGTTCCGCCCATTCTCCGCCCCATGGCTGACTAATTTTTTTTATTT
ATGCAGAGGCCGAGGCCGCCTCGGCCTCTGAGCTATTCCAGAAGTAGTGAGGAGGCTTTTTTGGAGGCCTAGGCT
TTTGCAAAAAGCTAACTTGTTTATTGCAGCTTATAATGGTTACAAATAAAGCAATAGCATCACAAATTTCACAAA
TAAAGCATTTTTTTCACTGCATTCTAGTTGTGGTTTGTCCAAACTCATCAATGTATCTTATCATGTCTGTCCGCT
TCCTCGCTCACTGACTCGCTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCAGCAAAAGGCCAGGAACCGT
AAAAAGGCCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGCGCTCTCCT
GTTCCGACCCTGCCGCTTACCGGATACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACTTATCGCCACTGGCAGCA
GCCACTGGTAACAGGATTAGCAGAGCGAGGTATGTAGGCGGTGCTACAGAGTTCTTGAAGTGGTGGCCTAACTAC
GGCTACACTAGAAGAACAGTATTTGGTATCTGCGCTCTGCTGAAGCCAGTTACCTTCGGAAAAAGAGTTGGTAGC
TCTTGATCCGGCAAACAAACCACCGCTGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTCAGTGGAACGAAAACTCACGTTAAGGG
ATTTTGGTCATGAGATTATCAAAAAGGATCTTCACCTAGATCCTTTTAAATTAAAAATGAAGTTTTAAATCAATC
TAAAGTATATATGAGTAAACTTGGTCTGACAGTTAGAAAAACTCATCGAGCATCAAATGAAACTGCAATTTATTC
ATATCAGGATTATCAATACCATATTTTTGAAAAAGCCGTTTCTGTAATGAAGGAGAAAACTCACCGAGGCAGTTC
CATAGGATGGCAAGATCCTGGTATCGGTCTGCGATTCCGACTCGTCCAACATCAATACAACCTATTAATTTCCCC
TCGTCAAAAATAAGGTTATCAAGTGAGAAATCACCATGAGTGACGACTGAATCCGGTGAGAATGGCAACAGCTTA
TGCATTTCTTTCCAGACTTGTTCAACAGGCCAGCCATTACGCTCGTCATCAAAATCACTCGCATCAACCAAACCG
TTATTCATTCGTGATTGCGCCTGAGCGAGACGAAATACGCGATCGCTGTTAAAAGGACAATTACAAACAGGAATC
GAATGCAACCGGCGCAGGAACACTGCCAGCGCATCAACAATATTTTCACCTGAATCAGGATATTCTTCTAATACC
TGGAATGCTGTTTTTCCGGGGATCGCAGTGGTGAGTAACCATGCATCATCAGGAGTACGGATAAAATGCTTGATG
GTCGGAAGAGGCATAAATTCCGTCAGCCAGTTTAGTCTGACCATCTCATCTGTAACATCATTGGCAACGCTACCT
TTGCCATGTTTCAGAAACAACTCTGGCGCATCGGGCTTCCCATACAATCGATAGATTGTCGCACCTGATTGCCCG
ACATTATCGCGAGCCCATTTATACCCATATAAATCAGCATCCATGTTGGAATTTAATCGCGGCCTAGAGCAAGAC
GTTTCCCGTTGAATATGGCTCATAACACCCCTTGTATTACTGTTTATGTAAGCAGACAGTTTTATTGTTCATGAT
GATATATTTTTATCTTGTGCAATGTAACATCAGAGATTTTGAGACACAACAATTGGTCGACGGATCC
SEQ ID NO: 23 pDNA3 plasmid pMD2.G (FIG. 21)
GGATCCCCTGAGGGGGCCCCCATGGGCTAGAGGATCCGGCCTCGGCCTCTGCATAAATAAAAAAAATTAGTCAGC
CATGAGCTTGGCCCATTGCATACGTTGTATCCATATCATAATATGTACATTTATATTGGCTCATGTCCAACATTA
CCGCCATGTTGACATTGATTATTGACTAGTTATTAATAGTAATCAATTACGGGGTCATTAGTTCATAGCCCATAT
ATGGAGTTCCGCGTTACATAACTTACGGTAAATGGCCCGCCTGGCTGACCGCCCAACGACCCCCGCCCATTGACG
TCAATAATGACGTATGTTCCCATAGTAACGCCAATAGGGACTTTCCATTGACGTCAATGGGTGGAGTATTTACGG
TAAACTGCCCACTTGGCAGTACATCAAGTGTATCATATGCCAAGTACGCCCCCTATTGACGTCAATGACGGTAAA
TGGCCCGCCTGGCATTATGCCCAGTACATGACCTTATGGGACTTTCCTACTTGGCAGTACATCTACGTATTAGTC
ATCGCTATTACCATGGTGATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGGGACTTTCCAAAATGTCGT
AACAACTCCGCCCCATTGACGCAAATGGGCGGTAGGCGTGTACGGTGGGAGGTCTATATAAGCAGAGCTCGTTTA
GTGAACCGTCAGATCGCCTGGAGACGCCATCCACGCTGTTTTGACCTCCATAGAAGACACCGGGACCGATCCAGC
CTCCCCTCGAAGCTTACATGTGGTACCGAGCTCGGATCCTGAGAACTTCAGGGTGAGTCTATGGGACCCTTGATG
TTTTCTTTCCCCTTCTTTTCTATGGTTAAGTTCATGTCATAGGAAGGGGAGAAGTAACAGGGTACACATATTGAC
CAAATCAGGGTAATTTTGCATTTGTAATTTTAAAAAATGCTTTCTTCTTTTAATATACTTTTTTGTTTATCTTAT
TTCTAATACTTTCCCTAATCTCTTTCTTTCAGGGCAATAATGATACAATGTATCATGCCTCTTTGCACCATTCTA
AAGAATAACAGTGATAATTTCTGGGTTAAGGCAATAGCAATATTTCTGCATATAAATATTTCTGCATATAAATTG
TAACTGATGTAAGAGGTTTCATATTGCTAATAGCAGCTACAATCCAGCTACCATTCTGCTTTTATTTTATGGTTG
GGATAAGGCTGGATTATTCTGAGTCCAAGCTAGGCCCTTTTGCTAATCATGTTCATACCTCTTATCTTCCTCCCA
CAGCTCCTGGGCAACGTGCTGGTCTGTGTGCTGGCCCATCACTTTGGCAAAGCACGTGAGATCTGAATTCAACAG
AGATCGATCTGTTTCCTTGACACTATGAAGTGCCTTTTGTACTTAGCCTTTTTATTCATTGGGGTGAATTGCAAG
TTCACCATAGTTTTTCCACACAACCAAAAAGGAAACTGGAAAAATGTTCCTTCTAATTACCATTATTGCCCGTCA
AGCTCAGATTTAAATTGGCATAATGACTTAATAGGCACAGCCATACAAGTCAAAATGCCCAAGAGTCACAAGGCT
ATTCAAGCAGACGGTTGGATGTGTCATGCTTCCAAATGGGTCACTACTTGTGATTTCCGCTGGTATGGACCGAAG
TATATAACACAGTCCATCCGATCCTTCACTCCATCTGTAGAACAATGCAAGGAAAGCATTGAACAAACGAAACAA
GGAACTTGGCTGAATCCAGGCTTCCCTCCTCAAAGTTGTGGATATGCAACTGTGACGGATGCCGAAGCAGTGATT
GTCCAGGTGACTCCTCACCATGTGCTGGTTGATGAATACACAGGAGAATGGGTTGATTCACAGTTCATCAACGGA
AAATGCAGCAATTACATATGCCCCACTGTCCATAACTCTACAACCTGGCATTCTGACTATAAGGTCAAAGGGCTA
TGTGATTCTAACCTCATTTCCATGGACATCACCTTCTTCTCAGAGGACGGAGAGCTATCATCCCTGGGAAAGGAG
GGCACAGGGTTCAGAAGTAACTACTTTGCTTATGAAACTGGAGGCAAGGCCTGCAAAATGCAATACTGCAAGCAT
TGGGGAGTCAGACTCCCATCAGGTGTCTGGTTCGAGATGGCTGATAAGGATCTCTTTGCTGCAGCCAGATTCCCT
GAATGCCCAGAAGGGTCAAGTATCTCTGCTCCATCTCAGACCTCAGTGGATGTAAGTCTAATTCAGGACGTTGAG
AGGATCTTGGATTATTCCCTCTGCCAAGAAACCTGGAGCAAAATCAGAGCGGGTCTTCCAATCTCTCCAGTGGAT
CTCAGCTATCTTGCTCCTAAAAACCCAGGAACCGGTCCTGCTTTCACCATAATCAATGGTACCCTAAAATACTTT
GAGACCAGATACATCAGAGTCGATATTGCTGCTCCAATCCTCTCAAGAATGGTCGGAATGATCAGTGGAACTACC
ACAGAAAGGGAACTGTGGGATGACTGGGCACCATATGAAGACGTGGAAATTGGACCCAATGGAGTTCTGAGGACC
AGTTCAGGATATAAGTTTCCTTTATACATGATTGGACATGGTATGTTGGACTCCGATCTTCATCTTAGCTCAAAG
GCTCAGGTGTTCGAACATCCTCACATTCAAGACGCTGCTTCGCAACTTCCTGATGATGAGAGTTTATTTTTTGGT
GATACTGGGCTATCCAAAAATCCAATCGAGCTTGTAGAAGGTTGGTTCAGTAGTTGGAAAAGCTCTATTGCCTCT
TTTTTCTTTATCATAGGGTTAATCATTGGACTATTCTTGGTTCTCCGAGTTGGTATCCATCTTTGCATTAAATTA
AAGCACACCAAGAAAAGACAGATTTATACAGACATAGAGATGAACCGACTTGGAAAGTAACTCAAATCCTGCACA
ACAGATTCTTCATGTTTGGACCAAATCAACTTGTGATACCATGCTCAAAGAGGCCTCAATTATATTTGAGTTTTT
AATTTTTATGGAATTCACCCCACCAGTGCAGGCTGCCTATCAGAAAGTGGTGGCTGGTGTGGCTAATGCCCTGGC
CCACAAGTTTCACTAAGCTCGCTTCCTTGCTGTCCAATTTCTATTAAAGGTTCCTTGGTTCCCTAAGTCCAACTA
CTAAACTGGGGGATATTATGAAGGGCCTTGAGCATCTGGATTCTGCCTAATAAAAAACATTTATTTTCATTGCAA
TGATGTATTTAAATTATTTCTGAATATTTTACTAAAAAGGGAATGTGGGAGGTCAGTGCATTTAAAACATAAAGA
AATGAAGAGCTAGTTCAAACCTTGGGAAAATACACTATATCTTAAACTCCATGAAAGAAGGTGAGGCTGCAAACA
GCTAATGCACATTGGCAACAGCCCTGATGCCTATGCCTTATTCATCCCTCAGAAAAGGATTCAAGTAGAGGCTTG
ATTTGGAGGTTAAAGTTTGGCTATGCTGTATTTTACATTACTTATTGTTTTAGCTGTCCTCATGAATGTCTTTTC
ACTACCCATTTGCTTATCCTGCATCTCTCAGCCTTGACTCCACTCAGTTCTCTTGCTTAGAGATACCACCTTTCC
CCTGAAGTGTTCCTTCCATGTTTTACGGCGAGATGGTTTCTCCTCGCCTGGCCACTCAGCCTTAGTTGTCTCTGT
TGTCTTATAGAGGTCTACTTGAAGAAGGAAAAACAGGGGGCATGGTTTGACTGTCCTGTGAGCCCTTCTTCCCTG
CCTCCCCCACTCACAGTGACCCGGAATCCCTCGACATGGCAGTCTAGCACTAGTGCGGCCGCAGATCTGCTTCCT
CGCTCACTGACTCGCTGCGCTCGGTCGTTCGGCTGCGGCGAGCGGTATCAGCTCACTCAAAGGCGGTAATACGGT
TATCCACAGAATCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACGCTCAAGTCAGA
GGTGGCGAAACCCGACAGGACTATAAAGATACCAGGCGTTTCCCCCTGGAAGCTCCCTCGTGCGCTCTCCTGTTC
CGACCCTGCCGCTTACCGGATACCTGTCCGCCTTTCTCCCTTCGGGAAGCGTGGCGCTTTCTCAATGCTCACGCT
GTAGGTATCTCAGTTCGGTGTAGGTCGTTCGCTCCAAGCTGGGCTGTGTGCACGAACCCCCCGTTCAGCCCGACC
GCTGCGCCTTATCCGGTAACTATCGTCTTGAGTCCAACCCGGTAAGACACGACTTATCGCCACTGGCAGCAGCCA
CTGGTAACAGGATTAGCAGAGCGAGGTATGTAGGCGGTGCTACAGAGTTCTTGAAGTGGTGGCCTAACTACGGCT
ACACTAGAAGGACAGTATTTGGTATCTGCGCTCTGCTGAAGCCAGTTACCTTCGGAAAAAGAGTTGGTAGCTCTT
GATCCGGCAAACAAACCACCGCTGGTAGCGGTGGTTTTTTTGTTTGCAAGCAGCAGATTACGCGCAGAAAAAAAG
GATCTCAAGAAGATCCTTTGATCTTTTCTACGGGGTCTGACGCTCAGTGGAACGAAAACTCACGTTAAGGGATTT
TGGTCATGAGATTATCAAAAAGGATCTTCACCTAGATCCTTTTAAATTAAAAATGAAGTTTTAAATCAATCTAAA
GTATATATGAGTAAACTTGGTCTGACAGTTACCAATGCTTAATCAGTGAGGCACCTATCTCAGCGATCTGTCTAT
TTCGTTCATCCATAGTTGCCTGACTCCCCGTCGTGTAGATAACTACGATACGGGAGGGCTTACCATCTGGCCCCA
GTGCTGCAATGATACCGCGAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTGCCGGGAAGCTAGAGTAA
GTAGTTCGCCAGTTAATAGTTTGCGCAACGTTGTTGCCATTGCTACAGGCATCGTGGTGTCACGCTCGTCGTTTG
GTATGGCTTCATTCAGCTCCGGTTCCCAACGATCAAGGCGAGTTACATGATCCCCCATGTTGTGCAAAAAAGCGG
TTAGCTCCTTCGGTCCTCCGATCGTTGTCAGAAGTAAGTTGGCCGCAGTGTTATCACTCATGGTTATGGCAGCAC
TGCATAATTCTCTTACTGTCATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATAATACCGCGCCACATAGCAGAA
CTTTAAAAGTGCTCATCATTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAGCGTTTCTGGGTGAGCAA
AAACAGGAAGGCAAAATGCCGCAAAAAAGGGAATAAGGGCGACACGGAAATGTTGAATACTCATACTCTTCCTTT
TTCAATATTATTGAAGCATTTATCAGGGTTATTGTCTCATGAGCGGATACATATTTGAATGTATTTAGAAAAATA
AACAAATAGGGGTTCCGCGCACATTTCCCCGAAAAGTGCCACCTGACGT
SEQ ID NO: 24 codon-optimised gag-pol genes

| | |
|---|---|
| atgggagctg ccacatctgc cctgaataga cggcagctgg accagttcga gaagatcaga | 60 |
| ctgcggccca acggcaagaa gaagtaccag atcaagcacc tgatctgggc cggcaaagag | 120 |
| atggaaagat tcggcctgca cgagcggctg ctggaaaccg aggaaggctg caagagaatt | 180 |
| atcgaggtgc tgtaccctct ggaacctacc ggctctgagg gcctgaagtc cctgttcaat | 240 |
| ctcgtgtgcg tgctgtactg cctgcacaaa gaacagaaag tgaaggacac cgaagaggcc | 300 |
| gtggccacag ttagacagca ctgccacctg gtggaaaaag agaagtccgc cacagagaca | 360 |
| agcagcggcc agaagaagaa cgacaaggga attgctgccc ctcctggcgg cagccagaat | 420 |
| tttcctgctc agcagcaggg aaacgcctgg gtgcacgttc cactgagccc tagaacactg | 480 |
| aatgcctggg tcaaagccgt ggaagagaag aagtttggcg ccgagatcgt gcccatgttc | 540 |
| caggctctgt ctgagggctg caccccttac gacatcaacc agatgctgaa cgtgctggga | 600 |
| gatcaccagg gcgctctgca gatcgtgaaa gagatcatca acgaagaggc tgcccagtgg | 660 |
| gacgtgacac atccattgcc tgctggacct ctgccagccg gacaactgag agatcctaga | 720 |
| ggctctgata tcgccggcac caccagctct gtgcaagagc agctggaatg gatctacacc | 780 |
| gccaatccta gagtggacgt gggcgccatc tacagaagat ggatcatcct gggcctgcag | 840 |
| aaatgcgtga agatgtacaa ccccgtgtcc gtgctggaca tcagacaggg acccaaagag | 900 |
| cccttcaagg actacgtgga ccggttctat aaggccatta gagccgagca ggccagcggc | 960 |
| gaagtgaagc agtggatgac agagagcctg ctgatccaga acgccaatcc agactgcaaa | 1020 |
| gtgatcctga aaggcctggg catgcacccc acactggaag agatgctgac agcctgtcaa | 1080 |
| ggcgttggcg gcccttctta caaagccaaa gtgatggccg agatgatgca gaccatgcag | 1140 |
| aaccagaaca tggtgcagca aggcggccct aagagacaga ggcctcctct gagatgctac | 1200 |
| aactgcggca agttcggcca catgcagaga cagtgtcctg agcctaggaa aacaaaatgt | 1260 |
| ctaaagtgtg gaaaattggg acacctagca aaagactgca ggggacaggt gaatttttta | 1320 |
| gggtatggac ggtggatggg ggcaaaaccg agaaattttc ccgccgctac tcttggagcg | 1380 |
| gaaccgagtg cgcctcctcc accgagcggc accaccccat acgacccagc aaagaagctc | 1440 |
| ctgcagcaat atgcagagaa agggaaacaa ctgagggagc aaaagaggaa tccaccggca | 1500 |
| atgaatccgg attggaccga gggatattct ttgaactccc tctttggaga agaccaataa | 1560 |
| agaccgtgta catcgagggc gtgcccatca aggctctgct ggatacaggc gccgacgaca | 1620 |
| ccatcatcaa agagaacgac ctgcagctga gcggcccttg gaggcctaag atcattggag | 1680 |
| gaatcggcgg aggcctgaac gtcaaagagt acaacgaccg ggaagtgaag atcgaggaca | 1740 |
| agatcctgag gggcacaatc ctgctgggcg ccacacctat caacatcatc ggcagaaatc | 1800 |
| tgctggcccc tgccggcgct agactggtta tgggacagct ctctgagaag atccccgtga | 1860 |
| cacccgtgaa gctgaaagaa ggcgctagag gaccttgtgt gcgacagtgg cctctgagca | 1920 |
| aagagaagat tgaggccctg caagaaatct gtagccagct ggaacaagag ggcaagatca | 1980 |
| gcagagttgg cggcgagaac gcctacaata cccctatctt ctgcatcaag aaaaaggaca | 2040 |
| agagccagtg gcggatgctg gtggacttta gagagctgaa caaggctacc caggacttct | 2100 |
| tcgaggtgca gctgggaatt cctcatcctg ccggcctgcg gaagatgaga cagatcacag | 2160 |
| tgctggatgt gggcgacgcc tactacagca tccctctgga ccccaacttc agaaagtaca | 2220 |
| ccgccttcac aatccccacc gtgaacaatc aaggccctgg catcagatac cagttcaact | 2280 |
| gcctgcctca aggctggaag ggcagcccca ccatttttca gaataccgcc gccagcatcc | 2340 |
| tggaagaaat caagagaaac ctgcctgctc tgaccatcgt gcagtacatg gacgatctgt | 2400 |
| gggtcggaag ccaagagaat gagcacaccc acgacaagct ggtggaacag ctgagaacaa | 2460 |
| agctgcaggc ctggggcctc gaaacccctg agaagaaggt gcagaaagaa cctccttacg | 2520 |
| agtggatggg ctacaagctg tggcctcaca agtgggagct gagccggatt cagctcgaag | 2580 |
| agaaggacga gtggaccgtg aacgacatcc agaaactcgt gggcaagctg aattgggcag | 2640 |
| cccagctgta tcccggcctg aggaccaaga acatctgcaa gctgatccgg ggaaagaaga | 2700 |
| acctgctgga actggtcaca tggacacctg aggccgaggc cgaatatgcc gagaatgccg | 2760 |
| aaatcctgaa aaccgagcaa gaggggacct actacaagcc tggcattcca atcagagctg | 2820 |
| ccgtgcagaa actggaaggc ggccagtggt cctaccagtt taagcaagaa ggccaggtcc | 2880 |
| tgaaagtggg caagtacacc aagcagaaga acacccacac caacgagctg aggacactgg | 2940 |
| ctggcctggt ccagaaaatc tgcaaagagg ccctggtcat ttggggcatc ctgcctgttc | 3000 |
| tggaactgcc cattgagcgg gaagtgtggg aacagtggtg ggccgattac tggcaagtgt | 3060 |
| cttggatccc cgagtgggac ttcgtgtcta cccctcctct gctgaaactg tggtacaccc | 3120 |
| tgacaaaaga gcccattcct aaagaggacg tctactacgt tgacggcgcc tgcaaccgga | 3180 |
| actccaaaga aggcaaggcc ggctacatca gccagtacgg caagcagaga gtggaaaccc | 3240 |
| tggaaaacac caccaaccag caggccgagc tgaccgccat taagatggcc ctggaagata | 3300 |
| gcggccccaa tgtgaacatc gtgaccgact ctcagtacgc catgggaatc ctgacagccc | 3360 |
| agcctacaca gagcgatagc cctctggttg agcagatcat tgccctgatg attcagaagc | 3420 |
| agcaaatcta cctgcagtgg gtgcccgctc acaaaggcat cggcggaaac gaagagatcg | 3480 |
| ataagctggt gtccaaggga atcagacggg tgctgttcct ggaaaagatt gaagaggccc | 3540 |
| aagaggaaca cgagcgctac cacaacaact ggaagaatct ggccgacacc tacggactgc | 3600 |
| cccagatcgt ggccaaagaa atcgtggcta tgtgccccaa gtgtcagatc aagggcgaac | 3660 |
| ctgtgcacgg ccaagtggat gcttctcctg gcacatggca gatggactgt acccacctgg | 3720 |
| aaggcaaagt ggtcatcgtg gctgtgcacg tggcctccgg ctttattgag gccgaagtga | 3780 |
| tccccagaga gacaggcaaa gaaaccgcca agttcctgct gaagatcctg tccagatggc | 3840 |
| ccatcacaca gctgcacacc gacaacggcc ctaacttcac atctcaagag gtggccgcca | 3900 |
| tctgttggtg gggaaagatt gagcacacaa ccggcattcc ctacaatcca cagagccagg | 3960 |
| gcagcatcga gtccatgaac aagcagctca aagagattat cggcaagatc cgggacgact | 4020 |
| gccagtacac agaaacagcc gtgctgatgg cctgtcacat ccacaacttc aagcggaaag | 4080 |
| gcggcatcgg aggacagaca tctgccgaga gactgatcaa tatcatcacc actcagctgg | 4140 |
| aaatccagca cctccagacc aagatccaga agattctgaa cttccgggtg tactaccgcg | 4200 |
| agggcagaga tcctgtttgg aaaggcccag cacagctgat ctggaaaggc gaaggtgccg | 4260 |
| tggtgctgaa ggatggctct gatctgaagg tggtgcccag acggaaggcc aagattatca | 4320 |
| aggattacga gcccaaacag cgcgtgggca atgaaggcga cgttgagggc acaagaggca | 4380 |
| gcgacaattg a | 4391 |

SEQ ID NO: 25 exemplary CAG promoter

| | |
|---|---|
| attgattatt gactagttat taatagtaat caattacggg gtcattagtt catagcccat | 60 |
| atatggagtt ccgcgttaca taacttacgg taaatggccc gcctggctga ccgcccaacg | 120 |
| acccccgccc attgacgtca ataatgacgt atgttcccat agtaacgcca atagggactt | 180 |
| tccattgacg tcaatgggtg gagtatttac ggtaaactgc ccacttggca gtacatcaag | 240 |
| tgtatcatat gccaagtacg ccccctattg acgtcaatga cggtaaatgg cccgcctggc | 300 |
| attatgccca gtacatgacc ttatgggact ttcctacttg gcagtacatc tacgtattag | 360 |
| tcatcgctat taccatggtc gaggtgagcc ccacgttctg cttcactctc cccatctccc | 420 |
| ccccctcccc acccccaatt ttgtatttat ttatttttta attattttgt gcagcgatgg | 480 |
| gggcgggggg gggggggggg cgcgcgccag gcggggcggg gcggggcgag gggcggggcg | 540 |
| gggcgaggcg gagaggtgcg gcggcagcca atcagagcgg cgcgctccga aagtttcctt | 600 |
| ttatggcgag gcggcggcgg cggcggccct ataaaaagcg aagcgcgcgg cgggcgggag | 660 |
| tcgctgcgcg ctgccttcgc cccgtgcccc gctccgccgc cgcctcgcgc cgcccgcccc | 720 |
| ggctctgact gaccgcgtta ctcccacagg tgagcgggcg ggacggccct tctcctccgg | 780 |
| gctgtaatta gcgcttggtt taatgacggc ttgtttcttt tctgtggctg cgtgaaagcc | 840 |
| ttgaggggct ccgggagggc cctttgtgcg gggggagcgg ctcggggggt gcgtgcgtgt | 900 |
| gtgtgtgcgt ggggagcgcc gcgtgcggct ccgcgctgcc cggcggctgt gagcgctgcg | 960 |
| ggcgcggcgc ggggctttgt gcgctccgca gtgtgcgcga ggggagcgcg gccgggggcg | 1020 |
| gtgccccgcg gtgcgggggg ggctgcgagg ggaacaaagg ctgcgtgcgg ggtgtgtgcg | 1080 |
| tgggggggtg agcagggggt gtgggcgcgt cggtcgggct gcaacccccc ctgcaccccc | 1140 |
| ctccccgagt tgctgagcac ggcccggctt cgggtgcggg gctccgtacg gggcgtggcg | 1200 |
| cggggctcgc cgtgccgggc ggggggtggc ggcaggtggg ggtgccgggc ggggcggggc | 1260 |
| cgcctcgggc cggggagggc tcgggggagg ggcgcggcgg cccccggagc gccggcggct | 1320 |
| gtcgaggcgc ggcgagccgc agccattgcc ttttatggta atcgtgcgag agggcgcagg | 1380 |
| gacttccttt gtcccaaatc tgtgcggagc cgaaatctgg gaggcgccgc cgcaccccct | 1440 |
| ctagcgggcg cggggcgaag cggtgcggcg ccggcaggaa ggaaatgggc ggggagggcc | 1500 |
| ttcgtgcgtc gccgcgccgc cgtccccttc tccctctcca gcctcggggc tgtccgcggg | 1560 |
| gggacggctg ccttcggggg ggacggggca gggcggggtt cggcttctgg cgtgtgaccg | 1620 |
| gcggctctag agcctctgct aaccatgttc atgccttctt ctttttccta cagctcctgg | 1680 |
| gcaacgtgct ggttattgtg ctgtctcatc attttggcaa agaattgctc gagccacc | 1738 |

SEQ ID NO: 26 exemplary CMV promoter

| | |
|---|---|
| gcattgatta ttgactagtt attaatagta atcaattacg gggtcattag ttcatagccc | 60 |
| atatatggag ttccgcgtta cataacttac ggtaaatggc ccgcctggct gaccgcccaa | 120 |
| cgacccccgc ccattgacgt caataatgac gtatgttccc atagtaacgc caatagggac | 180 |
| tttccattga cgtcaatggg tggagtattt acggtaaact gcccacttgg cagtacatca | 240 |
| agtgtatcat atgccaagtc cgccccctat tgacgtcaat gacggtaaat ggcccgcctg | 300 |
| gcattatgcc cagtacatga ccttacggga ctttcctact tggcagtaca tctacgtatt | 360 |
| agtcatcgct attaccatg | 379 |

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Prolonged and/or High Level of mGM-CSF Expression is Associated with Significant Histopathological Changes

GM-CSF has several functions within the endogenous immune/inflammatory cascade, including differentiation of granulocytes (neutrophils, eosinophils, and basophils) and monocytes/macrophages from precursors. As such, it is a tightly regulated protein and is therefore likely to have a well-defined efficacy/toxicity window.

Untreated PAP mice showed mild pulmonary inflammatory changes and evidence of alveolar thickening as well as a mild phenotype in the liver, both likely related to the lack of GM-CSF expression in these knockout mice. Transduction with a Glux-expressing control vector did not alter these baselines changes.

Lungs of GM-CSF knockout mice were treated at increasing doses of the rSIV.F/HN-mGM-CSF (1×10⁷-92×10⁷ TU/mouse). Histopathology was compared to wildtype mice (WT), untreated knockout mice (PAP) and knockout mice treated with a control vector (Glux, 24×10⁷ TU/mouse) two months after treatment (n=3-6 mice/group). Analysis was performed blinded and scored using a semi-quantitative scoring system. Scores are defined as: − absent, −/+ equivocal, ++ mild, +++ moderate, ++++ severe. PAM=pulmonary alveolar macrophage.

TABLE 1
Dose-related histopathological changes after pulmonary administration
of rSIV.F/HN-mGM-CSF to GM-CSF knockout mice

| | WT | PAP | Glux | 1 × 10⁷ | 5 × 10⁷ | 25 × 10⁷ | 92 × 10⁷ |
|---|---|---|---|---|---|---|---|
| Lung | | | | | | | |
| Distorted | − | − | − | − | − | +++ | ++++ |
| architecture | | | | | | | |
| Inflammatory cell | − | ++ | ++ | +++ | +++ | ++++ | ++++ |
| infiltration | | | | | | | |
| Alveolar wall | − | ++ | ++ | ++ | +++ | ++++ | ++++ |
| thickness | | | | | | | |
| PAM alveoli | − | − | − | ++ | +++ | ++++ | ++++ |
| PAM bronchi | − | − | − | − | ++ | +++ | ++++ |
| Neutrophils | − | − | − | − | −/+ | +++ | ++++ |
| bronchi | | | | | | | |
| Consolidation | − | − | − | ++ | ++ | ++++ | ++++ |
| Giant cells | − | − | − | ++ | −/+ | +++ | ++++ |
| Eosinophilic | − | − | −/+ | − | −/+ | −/+ | +++ |
| material | | | | | | | |
| Oedema | − | −/+ | − | − | − | −/+ | +++ |
| Liver | | | | | | | |
| Inflammatory cell | − | ++ | ++ | ++ | +++ | ++++ | +++ |
| infiltration | | | | | | | |
| Portal area | − | −/+ | − | − | −/+ | ++++ | ++ |
| inflammation | | | | | | | |
| Dilated | − | −/+ | ++ | − | −/+ | +++ | +++ |
| congested | | | | | | | |
| sinusoids | | | | | | | |
| Dilated | − | −/+ | − | − | ++ | +++ | +++ |
| congested | | | | | | | |
| blood vessels | | | | | | | |
| Kidney | | | | | | | |
| Inflammatory cell | ++ | ++ | ++ | +++ | +++ | +++ | +++ |
| infiltration | | | | | | | |
| Dilated blood | − | − | −/+ | ++ | ++ | ++ | ++ |
| vessels | | | | | | | |
| Fibrosis | −/+ | − | − | − | − | ++ | ++ |
| Eosinophilic | − | − | −/+ | ++ | ++ | − | − |
| material | | | | | | | |
| Cysts | ++ | − | − | − | −/+ | − | − |
| Spleen | | | | | | | |
| Clusters of | −/+ | −/+ | − | −/+ | ++ | −/+ | ++ |
| megakaryocytes | | | | | | | |
| Macrophages | − | −/+ | − | − | +++ | −/+ | − |

Given the prolonged expression characteristic of the rSIV.F/HN platform technology, it was next assessed whether longer-term expression of mGM-CSF in mice receiving 1×10⁷ TU/mouse would lead to histopathological changes. Lungs of GM-CSF knockout mice were treated with rSIV.F/HN-mGM-CSF or the Glux control lentivirus (1×10⁷ TU/mouse) (n=3-10 mice/group). Histopathology was compared between GM-CSF and Glux treated mice at 1 week to 9 months after treatment. Analysis was performed blinded and scored using a semi-quantitative scoring system. Scores defined as: − absent, −/+ equivocal, ++ mild, +++ moderate, ++++ severe. PAM=pulmonary alveolar macrophage. In the former group, we observed progressively more severe histopathological changes in the lungs 6-9 months after treatment, including tissue consolidation, inflammation, and increased numbers of pulmonary macrophages. In addition, at the 9-month time point, we also saw an accumulation of inflammatory cells and blood vessel dilation in the kidneys.

TABLE 2
Time-related histopathological changes after pulmonary administration
of rSIV.F/HN-mGM-CSF to GM-CSF knockout mice

| | 1 week | | 1-2 months | | 6 months | | 9 months | |
|---|---|---|---|---|---|---|---|---|
| | Glux | GM-CSF | Glux | GM-CSF | Glux | GM-CSF | Glux | GM-CSF |
| Lung | | | | | | | | |
| Distorted | − | − | − | − | − | − | − | − |
| architecture | | | | | | | | |
| Inflammatory cell | ++ | +++ | +++ | +++ | +++ | +++ | − | ++++ |
| infiltration | | | | | | | | |
| Alveolar wall | − | −/+ | − | −/+ | − | − | − | ++++ |
| thickness | | | | | | | | |
| PAM alveoli | −/+ | +++ | − | +++ | −/+ | ++++ | +++ | ++++ |
| PAM bronchi | − | +++ | − | −/+ | − | +++ | ++ | ++++ |
| Neutrophils | − | − | − | − | − | ++ | − | ++++ |
| bronchi | | | | | | | | |
| Consolidation | − | +++ | − | − | − | −/+ | − | ++++ |
| Giant cells | − | − | − | − | − | −/+ | − | ++ |
| Eosinophilic | − | − | − | − | −/+ | − | − | −/+ |
| material | | | | | | | | |
| Oedema | − | −/+ | − | − | − | − | − | −/+ |
| Kidney | | | | | | | | |
| Inflammatory cell | − | −/+ | −/+ | ++ | −/+ | ++ | ++ | +++ |
| infiltration | | | | | | | | |
| Dilated blood | − | − | ++ | − | −/+ | − | − | +++ |
| vessels | | | | | | | | |
| Fibrosis | − | − | − | − | − | − | − | − |
| Epithelial | − | − | − | − | − | − | − | − |
| loss tubules | | | | | | | | |
| Eosinophilic | − | − | − | − | − | − | − | − |
| material | | | | | | | | |
| Cysts | − | − | − | − | − | − | − | − |

To further define a suitable efficacy/toxicity window in this model, the histopathological analysis was extended to an 11-month time point in animals treated with 1×10⁵ and ×10⁶ TU/mouse (n=4-7/group). As noted above, mice treated with these comparatively low doses showed significant improvements in PAP biomarkers even though levels of vector-induced mGM-CSF levels were below the lower limit of detection for the assay. 11 months after treatment, analysis was performed blinded and determined using a semi-quantitative scoring system. Scores defined as: − absent, −/+ equivocal, ++ mild, +++ moderate, ++++ severe. PAM=pulmonary alveolar macrophage.

Histopathological analysis at this time point showed no or moderate histopathology at 1×10⁵ or 1×10⁶ TU/mouse, respectively (Table 3). We therefore were able to define a window of efficacy/toxicity in this murine model.

TABLE 3
Histopathological changes after pulmonary administration of
ultra-low doses of rSIV.F/HN-mGM-CSF to GM-CSF knockout mice

| | Glux | 1 × 10⁵ | 1 × 10⁶ |
|---|---|---|---|
| Lung | | | |
| Distorted architecture | − | − | − |
| Inflammatory cell infiltration | ++ | ++ | +++ |
| Alveolar wall thickness | ++ | − | − |
| PAM alveoli | − | − | ++ |
| PAM bronchi | − | −/+ | −/+ |
| Neutrophils bronchi | − | − | −/+ |
| Consolidation | − | − | − |
| Giant cells | − | − | − |
| Eosinophilic material | − | − | − |
| Oedema | − | − | − |
| Liver | | | |
| Inflammatory cell infiltration | ++ | − | +++ |
| Portal area inflammation | − | − | ++ |
| Dilated congested sinusoids | ++ | − | − |
| Dilated congested blood vessels | − | − | −/+ |
| Kidney | | | |
| Inflammatory cell infiltration | ++ | ++ | +++ |
| Dilated blood vessels | −/+ | − | ++ |
| Fibrosis | − | − | − |
| Eosinophilic material | −/+ | −/+ | ++ |
| Cysts | − | − | −/+ |
| Spleen | | | |
| Clusters of megakaryocytes | − | − | − |
| Macrophages | − | − | − |

### Example 2—Non-Viral GM-CSF Gene Therapy Leads to Sufficient mGM-CSF Expression to Ameliorate the PAP Phenotype and Expression Stops Before Toxicity is been Observed

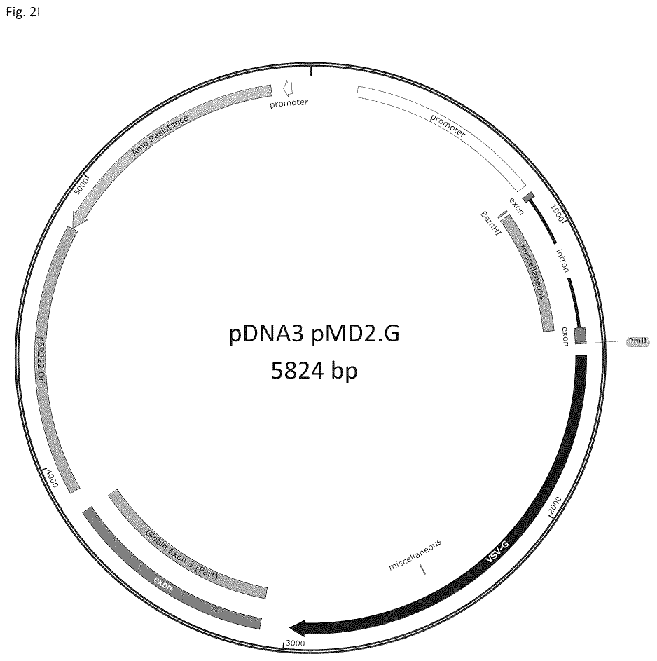

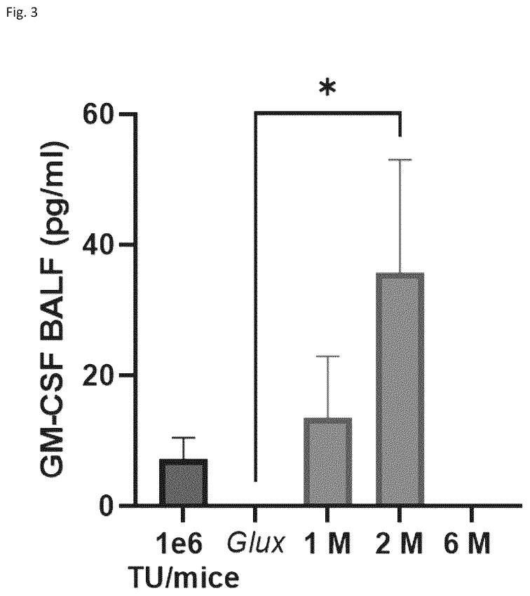

Mice were treated with an exemplary non-viral expression plasmid (phCEFI-GM-CSF) complexed to GL67A and GM-CSF expression was measured 1 month, 2 months and 6 months after single treatment. As shown in FIG. 3, GM-CSF expression was no longer detected at 6 months after treatment. Effective GM-CSF expression levels from the 1×10⁶ TU/mice lentivirus treated group was used for comparison. This experiment demonstrates that non-viral GM-CSF gene therapy may be used to deliver and express mGM-CSF at levels sufficient to ameliorate the PAP phenotype, and that expression stops before toxicity can result from protracted GM-CSF expression.

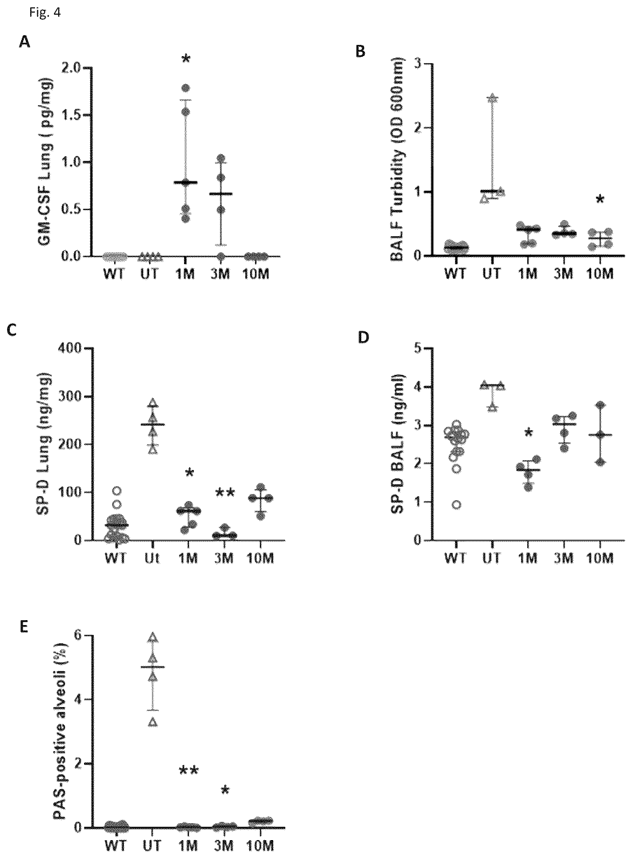

The possibility of a sustained treatment effect after a single dose of GL67A/mGM-CSFpDNA was further investigated. GM-CSF knockout mice were treated with GL67A-mGM-CSFpDNA complexes at a dose of 80 μg/mice. Untreated WT are included for reference. Animals were culled 1 to 10 months post-transfection and mGM-CSF expression was quantified in lung homogenate. As shown in FIG. 4A, the presence of mGM-CSF was observed in the 1 month and 3 month time points, but at 10 months mGM-CSF levels were undistinguishable from wild-type controls.

The effect of mGM-CSF expression on multiple biomarkers of PAP were analyzed. As shown in FIG. 4B, BALF turbidity remained observably decreased compared with the untransfected control, even at 10 months when mGM-CSF could no longer be detected in the lung homogenates. Similarly, surfactant protein D (SP-D) concentration in lung homogenate As shown in FIG. 4C) and BALF (FIG. 4D) remained observably reduced throughout the time course of the experiment, even when mGM-CSF expression in the lung homogenate was no longer detected. Lastly, surfactant deposition in the alveoli was quantified as percentage of PAS-positive alveoli. As shown in As shown in FIG. 4E, almost no surfactant deposition was observed across the whole time course following a single treatment with GL67A/mGM-CSFpDNA, even months after mGM-CSF ceased to be detectable.

### Example 3—Non-Viral GM-CSF Gene Therapy is Efficacious and Non-Toxic Over a Prolonged Period

The experiment of Example 2 was repeated, to investigate occurrence of histopathological changes in GM-CSF knockout mice treated with a single dose of GL67A/pDNA (80 μg/mouse), with any changes observed over a 10-month period. Animals were culled 1 to 10 months post-transfection with GL67A/pDNA. Histopathology analysis was performed blinded and determined using a semi-quantitative scoring system. As shown in Table 4, single dose GL67A/phCEFI-GM-CSF (plasmid DNA) gene transfer is well tolerated.

TABLE 4
Histopathological changes after pulmonary administration
of ultra-low doses of GL67A/pDNA to GM-CSF knockout mice

| Lung | WT | PAP | 1 M | 3 M | 10 M |
|---|---|---|---|---|---|
| Distorted architecture | − | − | − | − | − |
| Inflammatory cell infiltration | − | ++ | +/− | +/− | − |
| Alveolar wall thickness | − | − | − | − | − |
| PAM alveoli | − | − | − | − | − |
| PAM bronchi | − | − | − | − | − |
| Neutrophils bronchi | − | − | − | − | − |
| Consolidation | − | − | − | − | − |
| Giant cells | − | − | − | − | − |
| Eosinophilic material | − | − | − | − | − |
| Oedema | − | − | − | − | − |

Scores defined as: − absent, −/+ equivocal, ++ mild, +++ moderate, ++++ severe. PAM = pulmonary alveolar macrophage.

These data demonstrate that non-viral GM-CSF gene therapy (GL67A/phCEF-GM-CSF is well-tolerated.

### Example 4—Multiple Doses of Non-Viral GM-CSF can be Used to Express Therapeutic Levels of Free GM-CF

The KO GM-CSF mouse model lacks antibodies against GM-CSF. In a clinical setting, the presence of such antibodies could potentially lead to the clearance of GM-CSF, reducing the level of free GM-CSF and hence treatment efficacy.

Multidose treatments of other therapeutic proteins have been described in the art. However, it remained to be determined whether multiple doses of a GM-CSF vector could give rise to GM-CSF expression that would overcome any issues of antibody-driven clearance, whilst expressing GM-CSF at a level that would be therapeutically beneficial and without any histopathological changes. In other words, it was necessary to investigate whether multidose treatments with GM-CSF would deliver a free GM-CSF concentration within the narrow GM-CSF therapeutic window.

Accordingly, GM-CSF knockout mice's lungs were subjected to either a single dose (×1) or 5 doses (×5) of GL67A-mGM-CSFpDNA complexes (weekly doses of 80 μg/mice). Control mice received 5 doses of an irrelevant plasmid encoding a reporter gene (Luciferase). Animals were culled 1-month post-transfection, and mGM-CSF expression was quantified in lung homogenate (FIG. 5A). The impact of mGM-CSF expression on PAP biomarkers was analysed. In particular the surfactant protein D (SP-D) concentration in lung homogenate (FIG. 5A), BALF turbidity measured by absorbance (FIG. 5A) and surfactant deposition in alveoli quantified as a percentage of PAS-positive alveoli (FIG. 5A) were quantified.

As shown in FIG. 5A, multidose GL67A/phCEFI-GM-CSF (plasmid DNA) gene transfer led to heightened GM-CSF expression. In addition, a significant reduction in each of the PAP disease biomarkers quantified was observed. No histopathological changes were observed at 1 month post-completion of the multidose.

Therefore, multiple low doses of a GM-CSF plasmid have the potential to express GM-CSF at a therapeutically effective level within a subject, whilst the individual doses themselves are at low enough not to elicit any histopathological changes.

### Example 5—Viral GM-CSF Rene Therapy Leads to Sufficient mGM-CSF Expression to Ameliorate the PAP Phenotype and Expression Stops Before Toxicity is been Observed

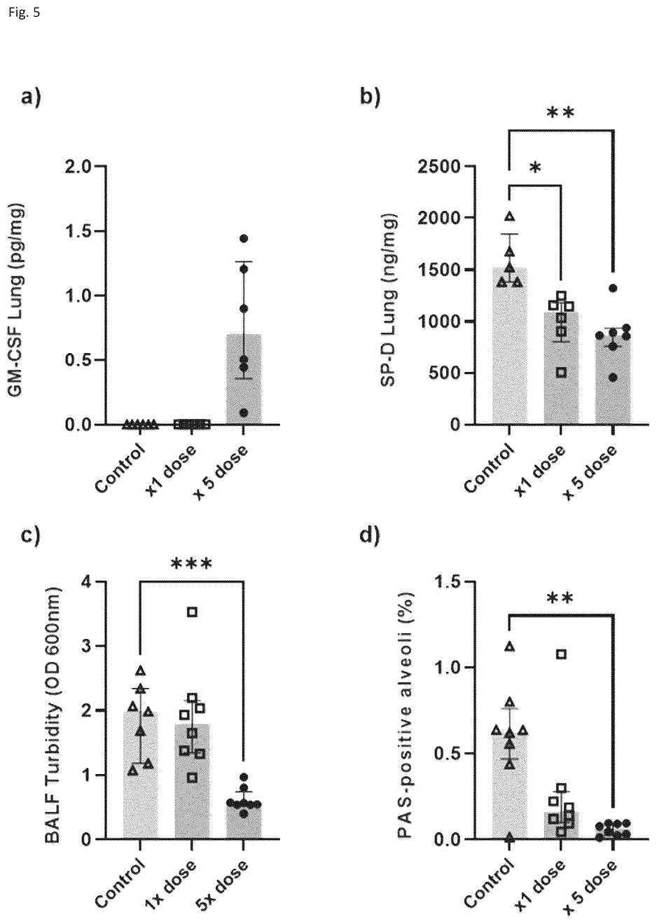

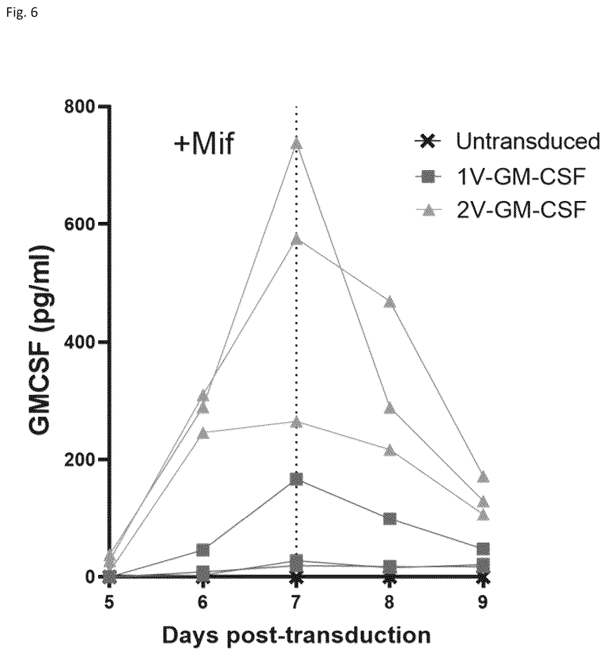

The potential utility of viral vectors for transiently expressing mGM-CSF was also investigated. Fully-differentiated human air liquid interface (ALI) cultures were transduced with 1V-GM-CSF MOI 100 and 2V-GM-CSF transgene:transactivator MOI 100:200 and transgene expression induced with 108M mifepristone for 48 hours. GM-CSF expression was measured in five consecutive daily apical washes. GMCSF expression increased by 27.8-fold in 1V cultures compared to 575.9-fold with 2V cultures. As shown in FIG. 6, both the 1 vector system (1V) and 2 vector system (2V), low concentrations (pg/mL) of mGM-CSF were transiently expressed, suggesting that viral vector systems with a GM-CSF transgene under the control of an inducible promoter may have potential utility in the treatment of PAP.

### Example 6—Viral GM-CSF Rene Expression can be Controlled by Promoter Selection to Achieve Expression within the Desired Therapeutic Window

The viral vector used in Example 1 uses the rSIV.F/HN-mGM-CSF, in which the mGM-CSF transgene is under the control of the hCEF promoter (CMV enhancer and elongation factor 1 alpha promoter), and which drives GM-CSF expression for a period of several months.

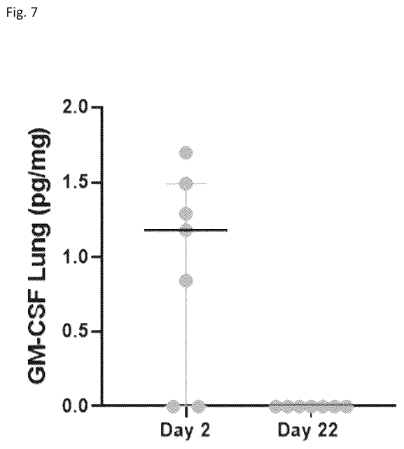

It was investigated whether selection of a different promoter could drive transient expression of GM-CSF. A pDNA comprising GM-CSF under the control of a CMV promoter and enhancer sequence was generated. Wild type mice were intranasally dosed with 80 μg of this pDNA, and culled at day 2 and day 22 following a single administration. GM-CSF expression was measured in the lung homogenate by Elisa and corrected by total protein. As shown in FIG. 7, the pDNA with the CMV promoter and enhancer sequence results in a reduced duration of GM-CSF expression. Expression was observed at day 2, but no GM-CSF expression was detectable 22 days post-dosing.

These data demonstrate that transient expression of GM-CSG within the desired therapeutic window can be achieved by selection of the promoter/enhancer elements in the plasmid DNA.

### Example 7—Generation of a Rodent Model for PAP

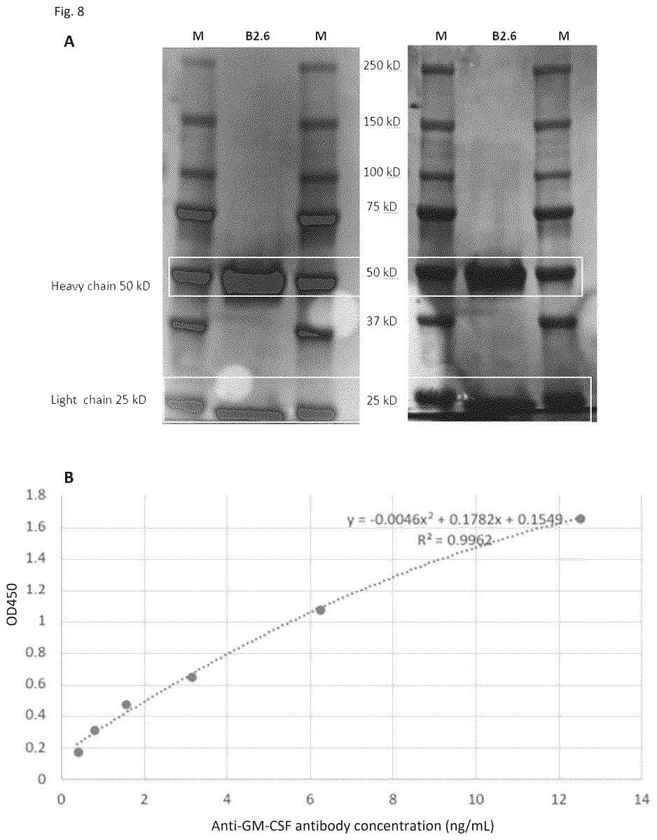

A monoclonal anti-mouse GM-CSF antibody (B2.6) was successfully purified from a hybridoma cell line (B2.6). As shown in FIG. 8A, the antibody exhibits both heavy and light chains, and was purified at a concentration of 820 ug/ml. The endotoxin levels of the purified antibody are below recommended levels for animal work (0.67 ng of endotoxin per mg of antibody).

A custom-made quantitative assay (ELISA) was developed to quantify the presence of B2.6 in mice following passive immunisation. 96 well plates were coated with purified murine (m)GM-CSF (1 ug/ml) over night and serial dilutions of purified B2.6 antibody were added to the well. A goat secondary anti-mouse IgG antibody conjugated to horseradish peroxidase (HRP) was used for visualisation and OD 450 was measured. As shown in FIG. 6B, the assay shows linearity over a broad range of antibody concentration (0.39-12.5 ng/ml).

Mice were treated with 328 ng and 82 ng by intraperitoneal and intranasal route, respectively. 24 hours or 7 days after passive immunisation mice were culled and anti-GM-CSF antibodies could be quantified in broncho-alveolar lavage fluid (treated mice OD=2.9, untreated mice OD=0.6). This demonstrates that passive immunisation of mice can deliver anti-GM-CSF antibodies at a level higher than those measured on the standard curve in FIG. 8B.

### Example 8—Optimisation of the Rodent Model for PAP

Following the development of the rodent model for PAP in Example 7, it was necessary to establish an antibody dose that would yield levels of the antibody in the epithelial lining fluid (ELF) of the lungs that was comparable to the median anti-GM-CSF antibody levels observed in aPAP patients.

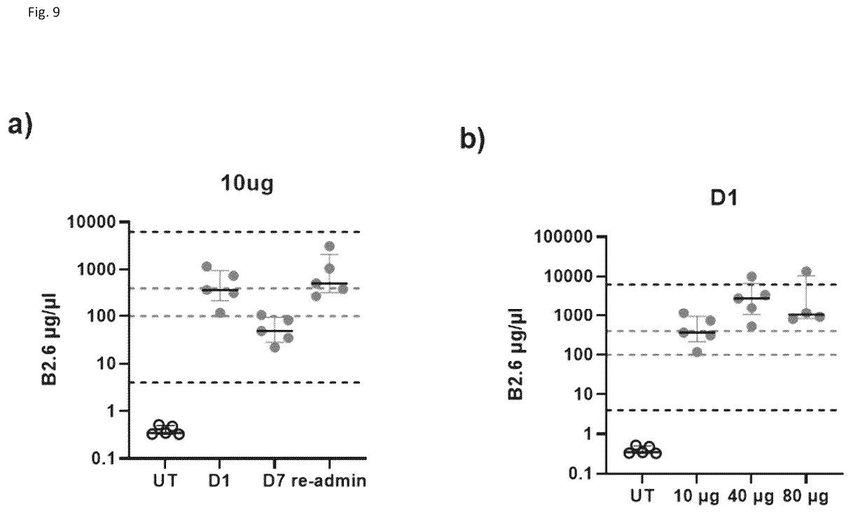

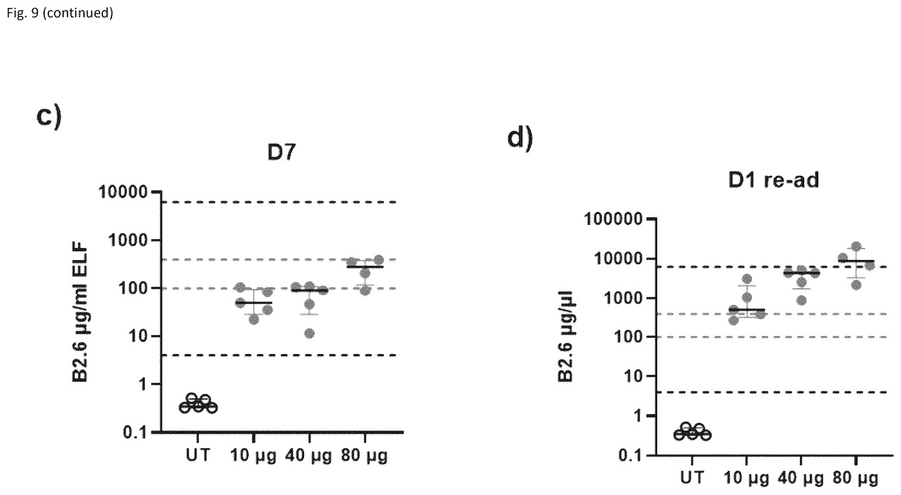

Wild-type (WT) mice were treated with different doses of B2.6 antibody 10, 40 or 80 μg/mice. Mice were culled 1 day (D1) or 7 days (D7) after single dose or 1 day after re-dosing (D1 re-admin). Antibody was detected in ELF by Elisa. As shown in FIG. 9a, a B2.6 antibody dose of 10 μg/mice yields detectable level of antibody in ELF akin to the median antibody levels found in aPAP patients (FIG. 9a).

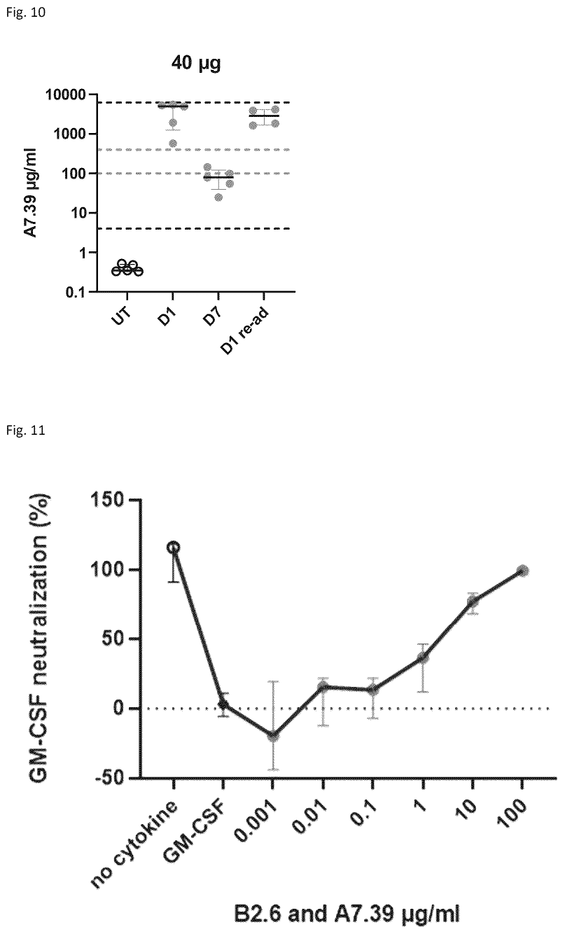

Similarly, as shown in FIG. 10, an A7.39 antibody dose of 40 μg/mice yields detectable level of antibody in ELF akin to the median antibody levels found in aPAP patients.

Detection of both anti GM-CSF antibodies (A7.39 and B2.6) was dose dependant, and they exhibit comparable half-lives. Consequently, as shown in the “D1 re-admin” group for both B2.6 and A7.39, re-dosing (weekly) was necessary to sustain the antibody levels in the ELF (FIG. 9 b-d and FIG. 10).

A functional bioassay was used to demonstrate that the antibody pair (A7.39 and B2.6) effectively neutralizes GM-CSF as shown in FIG. 11. FDC-P1 cells were seeded into 96-well plates (3000 cells/well). These cells require GM-CSF to proliferate. GM-CSF (200 pg/ml) was mixed with serial dilutions of B2.6 (0.001-100 ug/ml) and A7.39 (0.001-100 ug/ml) and added to the cells. Control cells were exposed to GM-CSF not mixed with antibodies. Cell proliferation was quantified after 3 days of culture using tetrazolium-based cell proliferation assay (Promega). GM-CSF neutralisation was calculated as percentage of inhibition of FDC-P1 growth with the following formula: [1−(OD of a single well−average OD of control cells grown without GM-CSF)×(average OD of control cells grown with GM-CSF−average OD of control cells grown without GM-CSF)−1]×100.

### Example 9—Formulations of GL67A/RNA Complexes for GM-CSF Expression

For most indications the short burst (a day or two) of protein expression achieved through mRNA-mediated transfection is a disadvantage. However, in the context of aPAP this likely represents an advantage in terms of the efficacy/toxicity window. In addition, GM-CSF mRNA transfer will support translation of the protein in the cytoplasm, thereby bypassing the nuclear membrane which presents a significant barrier for plasmids. In turn, this may allow for significantly smaller amounts of nucleic acids to be used to achieve GM-CSF expression compared to plasmid DNA.

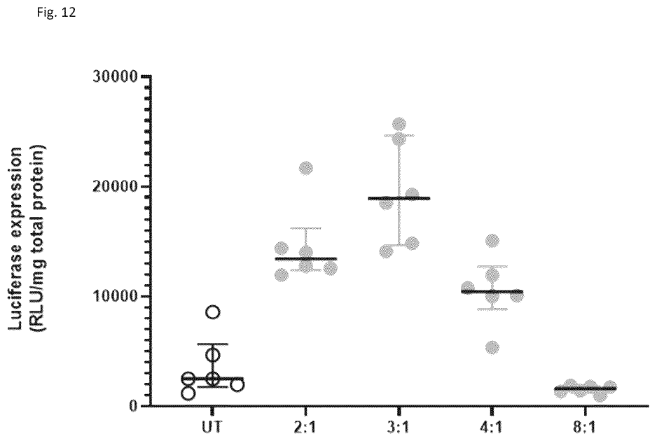

Accordingly, it was investigated whether GL67A could facilitate expression of mRNA expression. 293 T cells were transfected with mF-Luc (500 ng per well in 24-well plate) or remained UT as control (n=6 per group). After 8 hours incubation, cell lysates were harvested. Total protein concentration and luciferase expression were quantified. As shown in FIG. 12, GL67A was capable of delivering mRNA to 293 T cells, where it was expressed. These in vitro data suggests that GL67A:mRNA ratios in the range of from 2:1 to 4:1 may be particularly effective.

1. A granulocyte-macrophage colony-stimulating factor (GM-CSF) gene therapy agent for use in the treatment of pulmonary alveolar proteinosis (PAP), wherein said agent transiently expresses GM-CSF within a patient.

2. The agent for use according to claim 1, wherein transient GM-CSF protein expression is expression for six months or less, preferably 4 months or less, more preferably 3 months or less.

3. The agent for use according to claim 1 or 2, wherein said treatment reduces one or more PAP biomarker selected from:
(a) bronchoalveolar lavage fluid (BALF) turbidity;
(b) surfactant protein D (SF-D) concentration in the lungs;
(c) SF-D concentration in BALF;
(d) surfactant deposition in the lungs; and/or
(e) lung pathology, which is optionally selected from (i) pulmonary opacities, (ii) pulmonary oedema, and/or (iii) pulmonary consolidation;
and/or wherein said treatment increases lung function, which may optionally be selected from increasing (i) vital capacity (VC); (ii) forced vital capacity (FVC); and/or (iii) forced expiratory volume (FEV), particularly FEV1; (iv) arterial oxygen tension (Pa,O₂); (v) alveolar to arterial oxygen tension difference (PA-a,O₂); (vi) peak metabolic equivalents (peak METS) and/or (vii) 6-min walk distance (6 MWD), preferably PA-a,O₂.

4. The agent for use according to any one of claims 1 to 3, wherein said treatment is not associated with one or more histopathological change within the patient, said one or more histopathological change optionally being selected from the group consisting of:
(a) one or more histopathological change in the lungs, optionally distorted lung architecture, inflammatory cell infiltration of the lung above the PAP phenotype, increased alveolar wall thickness, pulmonary alveolar microlithiasis (PAM) alveoli, PAM bronchi, the presence of neutrophils in the bronchi, consolidation, the presence of giant cells, eosinophilic material and/or oedema;
(b) one or more histopathological change in the liver, optionally inflammatory cell infiltration above the PAP phenotype, portal area inflammation, dilated congested sinusoids and/or dilated congested blood vessels;
(c) one or more histopathological change in the kidneys, optionally inflammatory cell infiltration above the PAP phenotype, dilated blood vessels, fibrosis, eosinophilic material and/or cysts; and/or
(d) one or more histopathological change in the spleen, optionally clusters of megakaryocytes and/or the presence of macrophages.

5. The agent for use according to any one of the preceding claims, wherein said agent comprises:
(a) a non-viral nucleic acid molecule encoding GM-CSF, and a lipid carrier; or
(b) a viral vector or non-viral nucleic acid molecule comprising a GM-CSF transgene operably linked to an inducible promoter.

6. The agent for use of claim 5, wherein the non-viral nucleic acid molecule is a plasmid comprising a GM-CSF transgene operably linked to a promoter.

7. The agent for use of claim 6, wherein (i) the GM-CSF transgene; (ii) the promoter; or (iii) the GM-CSF transgene and the promoter; comprise 10 or fewer CpG dinucleotides, or are CpG dinucleotide free.

8. The agent for use of claim 6 or 7, wherein the plasmid comprises the GM-CSF transgene operably linked to a promoter selected from the group consisting of a hybrid human CMV enhancer/EF1a (hCEF) promoter, a cytomegalovirus (CMV) promoter, and elongation factor 1a (EF1a) promoter; optionally wherein the plasmid comprises a hybrid human CMV enhancer/EF1a (hCEF) promoter.

9. The agent for use of claim 5, wherein the non-viral nucleic acid molecule is an mRNA or a self-amplifying RNA (saRNA) encoding GM-CSF.

10. The agent for use of claim 9, wherein:
(a) the agent is an mRNA which comprises pseudouridine (ψ-UTP), a Cap1 and/or a poly(A) tail of between about 10 to 100 adenosine nucleotides, wherein optionally the mRNA is between about 0.5 kb to about 5 kb in length;
(b) the agent is an saRNA which comprises ψ-UTP, a Cap1 and/or a poly(A) tail of between about 10 to 100 adenosine nucleotides, wherein optionally the saRNA is between about 9 kb to about 12 kb in length.

11. The agent for use of any one of claims 5 to 10, wherein:
(a) the lipid carrier is a lipid nanoparticle, preferably a liposome;
(b) the lipid carrier comprises one or more cationic lipid, one or more non-cationic lipid, one or more cholesterol-based lipids and one or more PEG-modified lipids; and/or
(c) the lipid carrier is GL67A.

12. The agent for use of claim 5, wherein the viral vector is a lentiviral or retroviral vector.

13. The agent for use of claim 12, wherein the lentiviral or retroviral vector is:
(a) pseudotyped with (i) haemagglutinin-neuraminidase (HN) and fusion (F) proteins from a respiratory paramyxovirus, preferably from a Sendai virus, or (ii) or G glycoprotein from Vesicular Stomatitis Virus (G-VSV); and/or
(b) a lentiviral vector selected from the group consisting of a Simian immunodeficiency virus (SIV), a Human immunodeficiency virus (HIV) vector, vector, a Feline immunodeficiency virus (FIV) vector, an Equine infectious anaemia virus (EIAV) vector, and a Visna/maedi virus vector, preferably a SIV vector.

14. The agent for use of any one of claims 5, 12 or 13, wherein the inducible promoter is:
(a) (i) a steroid-regulated promoter, preferably a mifepristone-regulated promoter; or (ii) a chemically-regulated promoter; and/or
(b) (i) the transgene operably linked to an inducible promoter and the transactivator for the inducible promoter are comprised in (i) the same lentiviral or retroviral vector, or (ii) separate lentiviral or retroviral vectors.

15. The agent for use of any one of the preceding claims, which is formulated for administration to the lungs; optionally wherein the administration is by intratracheal or intranasal instillation, aerosol delivery, nebulization, intravenous injection, direct injection into the lungs.

16. The agent for use of any one of the preceding claims, wherein the PAP is autoimmune PAP (aPAP).

17. A method of treatment of PAP comprising administering a therapeutically effective amount of a GM-CSF gene therapy agent to a patient in need thereof.

18. Use of a GM-CSF gene therapy agent in the manufacture of a medicament for the treatment of PAP.

19. A composition comprising:
(a) a non-viral nucleic acid molecule encoding GM-CSF, and a lipid carrier; or
(b) an viral vector comprising a GM-CSF transgene operably linked to an inducible promoter;
and which is formulated for administration to the lungs, such that on administration said non-viral nucleic acid molecule or viral vector is capable of transiently expressing GM-CSF within cells of the lungs.

20. The composition of claim 19, wherein:
(a) the non-viral vector is a plasmid as defined in any one of claims 6 to 8; or
(b) the non-viral vector is mRNA or saRNA as defined in any one of claim 9 or 10;
and preferably the lipid carrier is as defined in claim 11.

21. A rodent model for aPAP, wherein said rodent has been passively immunised with anti-GM-CSF antibodies by intranasal administration.

22. The rodent model of claim 21, wherein:
(a) the rodent is a mouse, optionally a mouse with a C57 black 6 background, a wild-type mouse, or a GM-CSF knock out mouse;
(b) the anti-GM-CSF antibodies are murine anti-GM-CSF antibodies; and/or
(c) the model achieves a BALF concentration of anti GM-CSF antibodies of between about 4-6 μg/mL or greater.

23. A method of generating a rodent model for aPAP, comprising administration of anti-GM-CSF antibodies to a rodent by intranasal administration.

24. The method of claim 23, wherein:
(a) the rodent is a mouse, optionally a mouse with a C57 black 6 background, a wild-type mouse, or a GM-CSF knock out mouse; and/or
(b) the anti-GM-CSF antibodies are murine anti-GM-CSF antibodies; and/or
(c) the model achieves a BALF concentration of anti GM-CSF antibodies of between about 4-6 μg/mL or greater.

25. Use of a rodent model as defined in claim 21 or 22 for:
(a) studying aPAP;
(b) studying pharmaceuticals, cell products, biologics or small molecules intended for the treatment of aPAP, optionally studying compositions as defined in claim 19 or 20.
